(19) United States
(12) Patent Application Publication
(10) Pub. No.: US 2006/0079503 A1
(43) Pub. Date: Apr. 13, 2006

(54) Thiazolidinones and the use therof as polo-like kinase inhibitors

(75) Inventors: Wolfgang Schwede, Glienecke (DE); Volker Schulze, Berlin (DE); Knut Eis, Berlin (DE); Bernd Buchmann, Hohen Neuendorf (DE); Hans Briem, Bremen (DE); Gerhard Siemeister, Berlin (DE); Ulf Boemer, Glienecke (DE); Karsten Parczyk, Berlin (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: SCHERING AKTIENGESELLSCHAFT

(21) Appl. No.: 10/513,368

(22) PCT Filed: Apr. 29, 2003
(86) PCT No.: PCT/EP03/04450

§ 371 Date: Aug. 10, 2005

(30) Foreign Application Priority Data
May 3, 2002 (DE) 102 21 104.3

(51) Int. Cl. A61K 31/55; A61K 31/5377; A61K 31/496; A61K 31/454; A61K 31/426; C07D 417/02

(52) U.S. Cl. 514/217.1; 514/235.2; 514/254.02; 514/326; 514/369; 540/603; 544/369; 544/133; 546/209; 548/182

(57) ABSTRACT

Thiazolidones of general formula I 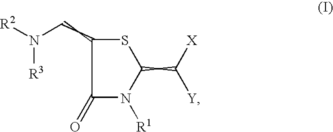
in which R¹, R², R³, X and Y have the meanings that are indicated in the description, their production and use as inhibitors of polo-like kinases (PLK) for treating various diseases as well as intermediate products for the production of thiazolidones are described.

The invention relates to thiazolidones, their production and use as inhibitors of polo-like kinase (Plk) for treating various diseases.

Tumor cells are distinguished by an uninhibited cell-cycle process. This is based on, on the one hand, the loss of control proteins, such as RB, p16, p21, p53 etc. as well as the activation of so-called accelerators of the cell-cycle process, the cyclin-dependent kinases (Cdks). The Cdks are an anti-tumor target protein that is acknowledged in pharmaceutics. In addition to the Cdks, serine/threonine kinases that regulate the new cell cycle, so-called ‘polo-like kinases,’ were described, which are involved not only in the regulation of the cell cycle but also in the coordination with other processes during mitosis and cytokinesis (formation of the spindle apparatus, chromosome separation). This class of proteins therefore represents an advantageous point of application for therapeutic intervention of proliferative diseases such as cancer (Descombes and Nigg. Embo J, 17; 1328 ff, 1998; Glover et al. Genes Dev 12, 3777 ff, 1998).

A high expression rate of Plk-1 was found in ‘non-small cell lung’ cancer (Wolf et al. Oncogene, 14, 543ff, 1997), in melanomas (Strebhardt et al. JAMA, 283, 479ff, 2000), in ‘squamous cell carcinomas’ (Knecht et al. Cancer Res, 59, 2794ff, 1999) and in ‘esophageal carcinomas’ (Tokumitsu et al. Int J Oncol 15, 687ff, 1999).

A correlation of a high expression rate in tumor patients with poor prognosis was shown for the most varied tumors (Strebhardt et al. JAMA, 283, 479ff, 2000, Knecht et al. Cancer Res, 59, 2794ff, 1999 and Tokumitsu et al. Int J Oncol 15, 687ff, 1999).

The constitutive expression of Plk-1 in NIH-3T3 cells resulted in a malignant transformation (increased proliferation, growth in soft agar, colony formation and tumor development in hairless mice (Smith et al. Biochem Biophys Res Comm, 234, 397ff., 1997).

Microinjections of Plk-1 antibodies in HeLa cells resulted in improper mitosis (Lane et al.; Journal Cell Biol, 135, 1701ff, 1996).

With a ‘20-mer’ antisense oligo, it was possible to inhibit the expression of Plk-1 in A549 cells, and to stop their ability to survive. It was also possible to show a significant anti-tumor action in hairless mice (Mundt et al., Biochem Biophys Res Comm, 269, 377ff., 2000).

The microinjection of anti-Plk antibodies in non-immortalized human Hs68 cells showed, in comparison to HeLa cells, a significantly higher fraction of cells, which remained in a growth arrest at G2 and showed far fewer signs of improper mitosis (Lane et al.; Journal Cell Biol, 135, 1701 ff, 1996).

In contrast to tumor cells, antisense-oligo-molecules inhibited the growth and the viability of primary human mesangial cells (Mundt et al., Biochem Biophys Res Comm, 269, 377ff., 2000).

In mammals, to date in addition to the Plk-1, three other polo-kinases were described that are induced as a mitogenic response and exert their function in the G1 phase of the cell cycle. These are, on the one hand, the so-called Prk/Plk-3 (the human homologue of the mouse-Fnk=fibroblast growth factor-induced kinase; Wiest et al, Genes, Chromosomes & Cancer, 32: 384ff, 2001), Snk/Plk-2 (serum-induced kinase, Liby et al., DNA Sequence, 11, 527-33, 2001) and sak/Plk4 (Fode et al., Proc. Natl. Acad. Sci. U.S.A., 91, 6388ff; 1994).

The inhibition of Plk-1 and the other kinases of the polo family, such as Plk-2, Plk-3 and Plk-4, thus represents a promising approach for the treatment of various diseases.

It has now been found that thiazolidones are suitable inhibitors of the kinases of the polo family.

The sequence identity within the Plk domains of the polo family is between 40 and 60%, so that partial interaction of inhibitors of a kinase occurs with one or more other kinases of this family. Depending on the structure of the inhibitor, however, the action can also take place selectively or preferably on only one kinase of the polo family.

The compounds according to the invention essentially inhibit the polo-like kinases, upon which is based their action against, for example, cancer, such as solid tumors and leukemia; auto-immune diseases, such as psoriasis, alopecia, and multiple sclerosis, chemotherapy-induced alopecia and mucositis; cardiovascular diseases, such as stenoses, arterioscleroses and restenoses; infectious diseases, such as, e.g., by unicellular parasites, such as trypanosoma, toxoplasma or plasmodium, or produced by fungi; nephrological diseases, such as, e.g., glomerulonephritis, chronic neurodegenerative diseases, such as Huntington's disease, amyotropic lateral sclerosis, Parkinson's disease, AIDS dementia and Alzheimer's disease; acute neurodegenerative diseases, such as ischemias of the brain and neurotraumas; viral infections, such as, e.g., cytomegalic infections, herpes, hepatitis B and C, and HIV diseases.

This invention thus relates to compounds of general formula I 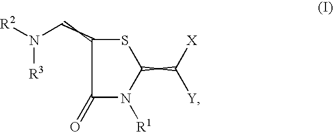
in which
X and Y are the same or different and stand for hydrogen, aryl, cyano, C₃-C₆-cycloalkyl or for the group —COOR⁴, —CONR¹⁵—(CH₂)ₙ—R²⁵, —COOR²⁵, —CONR¹⁵R¹⁶ or —COR¹³,
R¹, R¹¹, R¹²
R¹⁵, R¹⁶
R¹⁹ and R²⁰ are the same or different and stand for hydrogen, C₁-C₁₀-alkyl, C₂-C₁₀-alkenyl, C₂-C₁₀-alkinyl, (COOR¹⁴)—(CH₂)ₙ—, (C₃-C₆-cycloalkyl)-C₁-C₄-alkylene, C₃-C₆-cycloalkyl, phenylsulfonyl, phenyl-C₃-C₆-cycloalkyl, C₁-C₁₀-alkanoyl, C₁-C₆-alkoxy-C₁-C₆-alkylene, C₁-C₄-alkoxycarbonyl-C₁-C₄-alkylene, hydroxy-C₁-C₄-alkylene, —C₁-C₆-alkyl-O—Si(phenyl)₂—C₁-C₆-alkyl, or for the group COOR¹⁴, —COR¹³, —SO₂R¹⁸, —(CH₂)ₙ—NR¹⁵R¹⁶ or —(CH₂)ₙ—C(CH₃)_{q}—(CH₂)ₙNR¹⁵R¹⁶ or —NR¹¹R¹², or 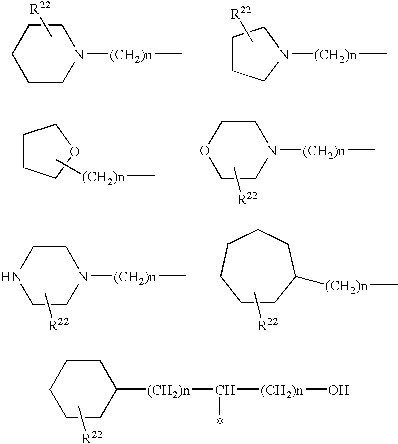
or for aryl, heteroaryl, heterocyclyl, aryl-C₁-C₄-alkylene, heteroaryl-C₁-C₄-alkylene, aryloxy-C₁-C₄-alkylene, heteroaryloxy-C₁-C₄-alkylene or aryl-C₁-C₄-alkylenoxy-C₁-C₄-alkylene that is optionally substituted in one or more places in the same way or differently with C₁-C₆-alkyl, C₂-C₆-alkenyl, C₂-C₆-alkinyl, C₃-C₆-cycloalkyl, C₃-C₆-cycloalkyloxy, phenyl, cyano, halogen, hydroxy, C₁-C₄-alkoxy, phenoxy, benzyloxy, C₁-C₄-alkylsulfanyl, benzylsulfanyl, phenylsulfanyl, dimethylamino, acetylamino, trifluoromethyl, trifluoromethoxy, trifluoromethylsulfanyl, acetyl, —CO—C₁-C₆-alkyl, 1-iminoethyl or nitro, or for C₁-C₁₀-alkyl that is substituted in one or more places with fluorine,
R² and R³ are the same or different and stand for hydrogen, C₁-C₆-alkyl, hydroxy-C₁-C₆-alkylene, C₃-C₆-cyclohexyl or for the group —COOR¹⁴, —CONR¹⁵R¹⁶, —COR¹³, —SO₂R¹⁸, —NR¹¹R¹², —(CH₂)ₙ-A, 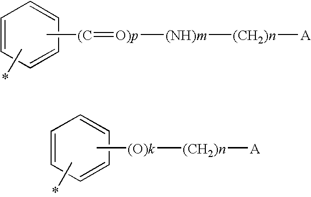
or for aryl, hetaroaryl or heterocyclyl that is optionally substituted in one or more places in the same way or differently with C₁-C₆-alkyl, C₃-C₆-cycloalkyl, halo-C₁-C₆-alkyl, halo-C₁-C₆-alkoxy, halogen, cyano, hydroxy-C₁-C₆-alkylene, hydroxy-C₁-C₆-alkylenoxy, aryl, heteroaryl, heterocyclyl, —C₁-C₆-alkyl-COOR⁸ or with the group —OR¹⁰, —COR¹³, —COOR¹⁴, —NR¹¹R¹², —NR¹¹—CO—NR¹¹R¹², —NR¹¹—CO—R¹³, —NR¹¹—SO₂—R¹³, —(CH₂)ₙ—CO—NR¹⁵R¹⁶, —SR¹⁰ or —SO₂R¹⁸,
R⁴, R⁸, R⁹, R¹⁰,
R¹³, R¹⁴, R¹⁷
and R¹⁸ are the same or different and stand for hydrogen, C₁-C₁₀-alkyl, hydroxy-C₁-C₆-alkylenoxy-C₁-C₆-alkylene, C₁-C₆-alkoxy-CO—C₁-C₆-alkylene, —(CH₂)ₙ—CO—NR¹⁵R¹⁶, C₂-C₁₀-alkenyl, C₂-C₁₀-alkinyl, (C₃-C₆-cycloalkyl)-C₁-C₄-alkylene, halo-C₁-C₆-alkyl, hydroxy-C₁-C₆-alkylene, (COOR¹⁴)—(CH₂)ₙ—, hydroxy-(CH₂)ₙ—O—(CH₂)ₙ, C₃-C₆-cycloalkyl, C₁-C₁₀-alkanoyl, or for the group —NR¹¹R¹², —(CH₂)ₙ—CO—R²⁵, —(CH₂)ₙ—NR¹⁵R¹⁶, COOR¹⁴—(CH₂)ₙ— or —COR¹³, or for aryl, heteroaryl, heterocyclyl, aryl-C₁-C₄-alkylene, heteroaryl-C₁-C₄-alkylene, aryloxy-C₁-C₄-alkylene, heteroaryloxy-C₁-C₄-alkylene or aryl-C₁-C₄-alkylenoxy-C₁-C₄-alkylene that is optionally substituted in one or more places in the same way or differently with C₁-C₆-alkyl, C₂-C₆-alkenyl, C₃-C₆-cycloalkyl, C₃-C₆-cycloalkyloxy, phenyl, cyano, halogen, hydroxy-C₁-C₆-alkyl, C₁-C₄-alkoxy, phenoxy, benzyloxy, C₁-C₄-alkylsulfanyl, benzylsulfanyl, phenylsulfanyl, dimethylamino, acetylamino, trifluoromethyl, trifluoromethoxy, trifluoromethylsulfanyl, acetyl, —CO—C₁-C₆-alkyl, 1-iminoethyl or nitro, or for C₁-C₁₀-alkyl that is substituted in one or more places with fluorine or for the group —NR¹¹R¹², —COR¹³, —SO₂R¹⁸, —(CH₂)ₙ—NR¹⁵R¹⁶, —(CH₂)ₙ—C(CH₃)_{q}—(CH₂)ₙNR¹⁵R¹⁶ or 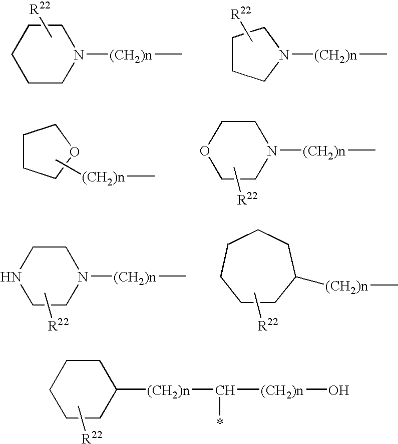
or
R² and R³,
R¹¹ and R¹²,
R¹⁵ and R¹⁶
and
R¹⁹ and R²⁰, in each case independently of one another, together form a 3- to 10-membered ring, which optionally can contain one or more nitrogen, oxygen or sulfur atoms,
or
R³ stands for hydrogen,
and
R² stands for the group -(L-M), in which
L stands for a group —C(O)—, —S(O)₂—, —C(O)N(R⁷)—, —S(O)₂ N(R⁷)—, —C(S)N(R⁷)—, —C(S)N(R⁷)C(O)O—, —C(O)O— or —C(O)S—,
and
M stands for hydrogen, C₁-C₁₀-alkyl, C₂-C₁₀-alkenyl, C₂-C₁₀-alkinyl, (C₃-C₆-cycloalkyl)-C₁-C₄-alkylene, C₃-C₆-cycloalkyl, phenyl-C₃-C₆-cycloalkyl, C₁-C₁₀-alkanoyl, C₁-C₄-alkoxy-C₁-C₄-alkylene, C₁-C₄-alkoxycarbonyl-C₁-C₄-alkylene, hydroxy-C₁-C₁₀-alkylene, or for aryl, heteroaryl, heterocyclyl, aryl-C₁-C₄-alkylene, heteroaryl-C₁-C₄-alkylene, aryloxy-C₁-C₄-alkylene, heteroaryloxy-C₁-C₄-alkylene or aryl-C₁-C₄-alkylenoxy-C₁-C₄-alkylene that is optionally substituted in one or more places in the same way or differently with C₁-C₄-alkyl, C₂-C₆-alkenyl, C₃-C₆-cycloalkyl, C₃-C₆-cycloalkyloxy, phenyl, cyano, halogen, phenoxy, benzyloxy, halo-C₁-C₄-alkoxy, halo-C₁-C₆-alkyl, nitro, —C₁-C₆-alkylCOOR⁸, —C₂-C₆-alkenylCOOR⁸, —C₂-C₆-alkinylCOOR⁸, —C₁-C₆-alkylOR⁹, —C₂-C₆-alkenylOR⁹, —C—C₆-alkinylOR⁹ or with the group —OR¹⁰, —NR¹¹R¹², —COR¹³, —COOR¹⁴, —CONR¹⁵R¹⁶, —SR¹⁷, —SO₂R¹⁸, SO₂NR¹⁹R²⁰ or —C(NH)(NH₂), or for C₁-C₁₀-alkyl that is substituted in one or more places with fluorine, and
R⁷ stands for hydrogen, C₁-C₁₀-alkyl, C₂-C₁₀-alkenyl, C₂-C₁₀-alkinyl, C₃-C₆-cycloalkyl, (C₃-C₆-cycloalkyl)-C₁-C₄-alkylene, aryl-C₁-C₄-alkylene,
A stands for optionally substituted aryl, heteroaryl or heterocyclyl,
R²² stands for hydrogen, hydroxy-C₁-C₆-alkyl, or for the group —OR¹⁰, —NR¹¹R¹², —COR¹³, —CONR¹⁵R¹⁶, —SO₂R¹⁸, —NR¹⁵—(C═S)—NR¹⁶—(CH₂)ₙ—R²⁴, —NR¹⁵—(C═O)—NR¹⁶—(CH₂)ₙ—R²⁴,
R²³ stands for hydrogen or C₁-C₆-alkyl,
R²⁴ stands for hydrogen, phenyl, C₁-C₆-alkoxy or for the group —(CH₂)ₙ—COO—C₁-C₆-alkyl,
R²⁵ stands for the group —OR¹⁰ or for C₂-C₆-alkenyl, phenyl, pyridyl, imidazolyl, morpholinyl, piperidinyl, C₃-C₆-cycloalkyl or 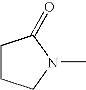
that is optionally substituted in one or more places in the same way or differently with halogen, C₁-C₆-alkyl, hydroxy-C₁-C₆-alkyl or with the group —OR¹⁰ or —COOR¹⁴.

m, p, and k, in each case independently of one another, stand for 0 or 1,
n stands for 0, 1, 2, 3, 4, 5, 6, 7, 8, 9 or 10,
q stands for 1 or 2, and their stereoisomers, mixtures of stereosiomers and their salts represent valuable compounds for inhibition of PLK and can be used in the above-indicated diseases.

Stereoisomers are defined as E/Z- and R/S-isomers as well as mixtures that consist of E/Z- and R/S-isomers.

Alkyl is defined in each case as a straight-chain or branched alkyl radical, such as, for example, methyl, ethyl, propyl, isopropyl, butyl, isobutyl, sec.-butyl, tert.-butyl, pentyl, isopentyl, hexyl, heptyl, octyl, nonyl and decyl.

Alkoxy is defined in each case as a straight-chain or branched alkoxy radical, such as, for example, methyloxy, ethyloxy, propyloxy, isopropyloxy, butyloxy, isobutyloxy, sec.-butyloxy, pentyloxy, isopentyloxy, hexyloxy, heptyloxy, octyloxy, nonyloxy or decyloxy.

The alkenyl substituents in each case are straight-chain or branched, and, for example, the following radicals are meant: vinyl, propen-1-yl, propen-2-yl, but-1-en-1-yl, but-1-en-2-yl, but-2-en-1-yl, but-2-en-2-yl, 2-methyl-prop-2-en-1-yl, 2-methyl-prop-1-en-1-yl, but-1-en-3-yl, but-3-en-1-yl, and allyl.

Alkinyl is defined in each case as a straight-chain or branched alkinyl radical, which contains 2-6, preferably 2-4 C atoms. For example, the following radicals can be mentioned: acetylene, propin-1-yl, propin-3-yl, but-1-in-1-yl, but-1-in-4-yl, but-2-in-1-yl, but-1-in-3-yl, etc.

Heterocyclyl stands for an alkyl ring that comprises 3-12 carbon atoms, which instead of carbon contains one or more heteroatoms, the same or different, such as, e.g., oxygen, sulfur or nitrogen, and can contain another substituent on one or more carbon or nitrogen atoms. Substituents on carbon can be═O, —OH, —C₁-C₄-hydroxyalkyl, alkyl, or CONR¹⁵R¹⁶. Substituents on nitrogen can be alkyl, COR¹³, —COOR¹⁴, —CONR¹⁵R¹⁶, —SO₂R¹⁸, or SO₂NR¹⁹R²⁰.

As heterocyclyls, there can be mentioned, e.g.: oxiranyl, oxethanyl, aziridinyl, azetidinyl, tetrahydrofuranyl, pyrrolidinyl, dioxolanyl, imidazolidinyl, pyrazolidinyl, dioxanyl, piperidinyl, morpholinyl, dithianyl, thiomorpholinyl, piperazinyl, trithianyl, quinuclidinyl, pyrolidonyl, N-methylpyrolidinyl, 2-hydroxymethylpyrolidinyl, 3 -hydroxypyrolidinyl, N-methylpiperazinyl, N-acetylpiperazinyl, N-methylsulfonylpiperazinyl, 4-hydroxypiperidinyl, 4-aminocarbonylpiperidinyl, 2-hydroxyethylpiperidinyl, 4-hydroxymethylpiperidinyl, etc.

Cycloalkyl is defined in each case as cyclopropyl, cyclobutyl, cyclopentyl and cyclohexyl.

Cycloalkyls are defined as monocyclic alkyl rings, such as cyclopropyl, cyclobutyl, cyclopentyl, cyclohexyl or cycloheptyl, but also bicyclic rings or tricyclic rings, such as, for example, adamantanyl.

The common portions of a 3- to 8-membered saturated, partially saturated or unsaturated ring are defined as ring systems in which optionally one or more possible double bonds can be contained in the ring, such as, for example, cycloalkenyls such as cyclopropenyl, cyclobutenyl, cyclopentenyl, cyclohexenyl, cycloheptenyl, or cyclooctenyl, whereby the linkage both to the double bond and to the single bonds can be carried out.

Halogen is defined in each case as fluorine, chlorine, bromine or iodine.

The aryl radical in each case has 6-12 carbon atoms, such as, for example, naphthyl, biphenyl and in particular phenyl.

In each case, the heteroaryl radical comprises 3-16 ring atoms and instead of carbon, can contain one or more heteroatoms, the same or different, such as oxygen, nitrogen or sulfur in the ring, and can be mono-, bi- or tricyclic, and can in addition in each case be benzocondensed.

For example, there can be mentioned: thienyl, furanyl, pyrrolyl, oxazolyl, thiazolyl, imidazolyl, pyrazolyl, isoxazolyl, isothiazolyl, oxadiazolyl, triazolyl, thiadiazolyl, etc., and benzo derivatives thereof, such as, e.g., benzofuranyl, benzothienyl, benzoxazolyl, benzimidazolyl, indazolyl, indolyl, isoindolyl, etc.; or pyridyl, pyridazinyl, pyrimidinyl, pyrazinyl, triazinyl, etc., and benzo derivatives thereof, such as, e.g., quinolyl, isoquinolyl, etc.; or oxepinyl, azocinyl, indolizinyl, indolyl, isoindolyl, indazolyl, benzimidazolyl, purinyl, etc., and benzo derivatives thereof, or quinolinyl, isoquinolinyl, cinnolinyl, phthalazinyl, quinazolinyl, quinoxalinyl, naphthyridinyl, pteridinyl, carbazolyl, acridinyl, phenazinyl, phenothiazinyl, phenoxazinyl, xanthenyl, etc.

Preferred heteroaryl radicals, are, for example, 5-ring heteroaromatic compounds, such as thiophene, furan, oxazole, thiazole, imidazole and benzo derivatives thereof, and 6-ring-heteroaromatic compounds, such as pyridine, pyrimidine, triazine, quinoline, isoquinoline and benzo derivatives thereof.

The aryl radical comprises 3-12 carbon atoms in each case and can be benzocondensed in each case.

For example, there can be mentioned: cyclopropenyl, cyclopentadienyl, phenyl, tropyl, cyclooctadienyl, indenyl, naphthyl, azulenyl, biphenyl, fluorenyl, anthracenyl, etc.

If an acid group is included, the physiologically compatible salts of organic and inorganic bases are suitable as salts, such as, for example, the readily soluble alkali and alkaline-earth salts, as well as N-methyl-glucamine, dimethyl-glucamine, ethyl-glucamine, lysine, 1,6-hexadiamine, ethanolamine, glucosamine, sarcosine, serinol, tris-hydroxy-methyl-amino-methane, aminopropane diol, Sovak base, and 1-amino-2,3,4-butanetriol.

If a basic group is included, the physiologically compatible salts of organic and inorganic acids are suitable, such as hydrochloric acid, sulfuric acid, phosphoric acid, citric acid, tartaric acid, i.a.

The compounds of general formula I according to the invention also contain the possible tautomeric forms and include the E- or Z-isomers, or, if a chiral center is present, also the racemates and enantiomers. Also encompassed are the double-bond isomers.

Preferred are those compounds of general formula I, in which
X and Y are the same or different and stand for hydrogen, phenyl, cyano, C₃-C₆-cycloalkyl or for the group —COOR⁴, —CONR¹⁵—(CH₂)ₙ—R²⁵, —COOR²⁵, —CONR¹⁵R¹⁶ or —COR¹³,
R¹, R¹¹, R¹²
R¹⁵, R¹⁶
R¹⁹ and R²⁰ are the same or different and stand for hydrogen, C₁-C₁₀-alkyl, C₂-C₁₀-alkenyl, C₂-C₁₀-alkinyl, (COOR¹⁴)—(CH₂)ₙ—, (C₃-C₆-cycloalkyl)-C₁-C₄-alkylene, C₃-C₆-cycloalkyl, phenylsulfonyl, phenyl-C₃-C₆-cycloalkyl, C₁-C₁₀-alkanoyl, C₁-C₆-alkoxy-C₁-C₆-alkylene, C₁-C₄-alkoxycarbonyl-C₁-C₄-alkylene, hydroxy-C₁-C₄-alkylene, —C₁-C₆-alkyl-O—Si(phenyl)₂—C₁-C₆-alkyl, or for the group COOR¹⁴, —COR¹³, —SO₂R¹⁸, —(CH₂)ₙ—NR¹⁵R¹⁶ or —(CH₂)ₙ—C(CH₃)_{q}—(CH₂)ₙNR¹⁵R¹⁶ or —NR¹¹R¹², or 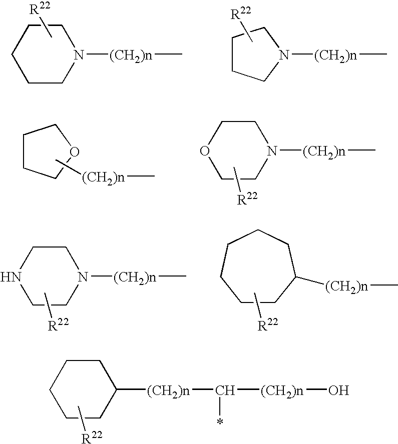
or for aryl, heteroaryl, heterocyclyl, aryl-C₁-C₄-alkylene, heteroaryl-C₁-C₄-alkylene, aryloxy-C₁-C₄-alkylene, heteroaryloxy-C₁-C₄-alkylene or aryl-C₁-C₄-alkylenoxy-C₁-C₄-alkylene that is optionally substituted in one or more places in the same way or differently with C₁-C₆-alkyl, C₂-C₆-alkenyl, C₂-C₆-alkinyl, C₃-C₆-cycloalkyl, C₃-C₆-cycloalkyloxy, phenyl, cyano, halogen, hydroxy, C₁-C₄-alkoxy, phenoxy, benzyloxy, C₁-C₄-alkylsulfanyl, benzylsulfanyl, phenylsulfanyl, dimethylamino, acetylamino, trifluoromethyl, trifluoromethoxy, trifluoromethylsulfanyl, acetyl, —CO—C₁-C₆-alkyl, 1-iminoethyl or nitro or for C₁-C₁₀-alkyl that is substituted in one or more places with fluorine,
R² and R³ are the same or different and stand for hydrogen, C₁-C₆-alkyl, hydroxy-C₁-C₆-alkylene, C₃-C₆-cyclohexyl or for the group —COOR¹⁴, —CONR¹⁵R¹⁶, —COR¹³, —SO₂R¹⁸, —NR¹¹R¹², —(CH₂)ₙ-A, 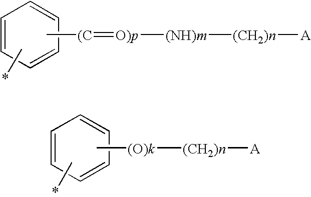
or for aryl, heteroaryl or heterocyclyl that is optionally substituted in one or more places in the same way or differently with C₁-C₆-alkyl, C₃-C₆-cycloalkyl, halo-C₁-C₆-alkyl, halo-C₁-C₆-alkoxy, halogen, cyano, hydroxy-C₁-C₆-alkylene, hydroxy-C₁-C₆-alkylenoxy, aryl, heteroaryl, heterocyclyl, —C₁-C₆-alkyl-COOR⁸ or with the group —OR¹⁰, —COR¹³, —COOR¹⁴, —NR¹¹R¹², —NR¹¹—CO—NR¹¹R¹², —NR¹¹—CO—R¹³, —NR¹¹—SO₂—R¹³, (CH₂)ₙ—CO—NR¹⁵R¹⁶, —SR¹⁰ or —SO₂R¹⁸,
R⁴, R⁸, R⁹,
R¹⁰, R¹³,
R¹⁴, R¹⁷
and R¹⁸ are the same or different and stand for hydrogen, C₁-C₁₀-alkyl, hydroxy-C₁-C₆-alkylenoxy-C₁-C₆-alkylene, C₁-C₆-alkoxy-CO—C₁-C₆-alkylene, —(CH₂)ₙ—CO—NR¹⁵R¹⁶, C₂-C₁₀-alkenyl, C₂-C₁₀-alkinyl, (C₃-C₆-cycloalkyl)-C₁-C₄-alkylene, halo-C₁-C₆-alkyl, hydroxy-C₁-C₆-alkylene, (COOR¹⁴)—(CH₂)ₙ—, hydroxy-(CH₂)ₙ—O—(CH₂)ₙ, C₃-C₆-cycloalkyl, C₁-C₁₀-alkanoyl, or for the group —NR¹¹R¹², —(CH₂)ₙ—CO—R²⁵, —(CH₂)ₙ—NR¹⁵R¹⁶, COOR¹⁴—(CH₂)ₙ— or —COR¹³, or for aryl, heteroaryl, heterocyclyl, aryl-C₁-C₄-alkylene, heteroaryl-C₁-C₄-alkylene, aryloxy-C₁-C₄-alkylene, heteroaryloxy-C₁-C₄-alkylene or aryl-C₁-C₄-alkylenoxy-C₁-C₄-alkylene that is optionally substituted in one or more places in the same way or differently with C₁-C₆-alkyl, C₂-C₆-alkenyl, C₃-C₆-cycloalkyl, C₃-C₆-cycloalkyloxy, phenyl, cyano, halogen, hydroxy-C₁-C₆-alkyl, C₁-C₄-alkoxy, phenoxy, benzyloxy, C₁-C₄-alkylsulfanyl, benzylsulfanyl, phenylsulfanyl, dimethylamino, acetylamino, trifluoromethyl, trifluoromethoxy, trifluoromethylsulfanyl, acetyl, —CO—C₁-C₆-alkyl, 1-iminoethyl or nitro, or for C₁-C₁₀-alkyl that is substituted in one or more places with fluorine or for the group —NR¹¹R¹², —COR¹³, —SO₂R¹⁸, —(CH₂)ₙ—NR¹⁵R¹⁶, —(CH₂)ₙ—C(CH₃)_{q}—(CH₂)ₙNR¹⁵R¹⁶ or 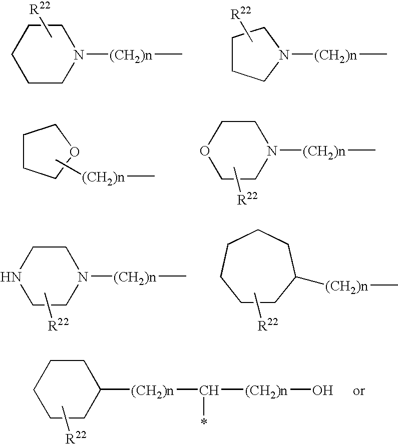
R² and R³,
R¹¹ and R¹²,
R¹⁵ and R¹⁶
and
R¹⁹ and R²⁰, in each case independently of one another, together form a 3- to 10-membered ring, which optionally can contain one or more nitrogen, oxygen or sulfur atoms,
A stands for optionally substituted aryl, heteroaryl or heterocyclyl,
R²² stands for hydrogen, hydroxy-C₁-C₆-alkyl, or for the group —OR¹⁰, —NR¹¹R¹², COR¹³, —CONR¹⁵R¹⁶, —SO₂R¹⁸, —NR¹⁵—(C═S)—NR¹⁶—(CH₂)ₙ—R²⁴, NR¹⁵—(C═O)—NR¹⁶—(CH₂)ₙ—R²⁴,
R²³ stands for hydrogen or C₁-C₆-alkyl,
R²⁴ stands for hydrogen, phenyl, C₁-C₆-alkoxy or for the group —(CH₂)ₙ—COO—C₁-C₆-alkyl,
R²⁵ stands for the group —OR¹⁰ or for C₂-C₆-alkenyl, phenyl, pyridyl, imidazolyl, morpholinyl, piperidinyl, C₃-C₆-cycloalkyl or 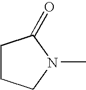
that is optionally substituted in one or more places in the same way or differently with halogen, C₁-C₆-alkyl, hydroxy-C₁-C₆-alkyl or with the group —OR¹⁰ or —COOR¹⁴,
m, p, k, in each case independently of one another, stand for 0 or 1,
n stands for 0, 1, 2, 3, 4, 5, 6, 7, 8, 9 or 10,
q stands for 1 or 2, as well as their stereoisomers, mixtures of the stereoisomers and their salts.

Selected compounds are those compounds of general formula I, in which
X and Y are the same or different and stand for hydrogen, phenyl, cyano, C₃-C₆-cycloalkyl or for the group —COOR⁴, —CONR¹⁵—(CH₂)ₙ—R²⁵, —COOR²⁵, —CONR¹⁵R¹⁶ or —COR¹³,
R¹ stands for hydrogen, phenyl, C₁-C₆-alkyl, C₃-C₆-cycloalkyl, hydroxy-C₁-C₄-alkylene, C₁-C₆-alkoxy-C₁-C₆-alkylene or for the group —C₁-C₆-alkyl-O—Si(phenyl)₂—C₁-C₆-alkyl,
R² and R³ are the same or different and stand for hydrogen, C₁-C₆-alkyl, hydroxy-C₁-C₄-alkylene, cyclohexyl or for the group —COOR¹⁴, —CONR¹⁵R¹⁶, —COR¹³, —SO₂R¹⁸, —NR¹¹R¹², —(CH₂)ₙ-A 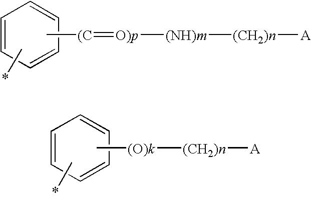
or for phenyl, pyridyl, naphthyl, biphenyl, imidazolyl, indazolyl, isothiazolyl, triazolyl, benztriazolyl, quinolinyl, isoquinolinyl, thiazolyl, pyrazolyl, anthrazenyl, pyrazolidinyl, oxazolyl, phthalazinyl, carbazolyl, benzimidazolyl, benzthiazolyl, isoxazolyl, indanyl, indolyl, pyrimidinyl, thiadiazolyl 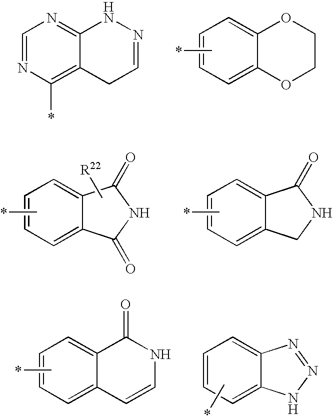
that is optionally substituted in one or more places in the same way or differently with C₁-C₆-alkyl, C₃-C₆-cycloalkyl, halo-C₁-C₆-alkyl, halo-C₁-C₆-alkoxy, halogen, cyano, triazolyl, tetrazolyl, hydroxy-C₁-C₆-alkylene, hydroxy-C₁-C₆-alkylenoxy, morpholino, —C₁-C₆-alkyl-COOR⁸ or with the group —OR¹⁰, —COR¹³, —COOR¹⁴, —NR¹¹R¹², —NR¹¹—CO—NR¹¹R¹², —NR¹¹—CO—R¹³, —NR¹¹—SO₂—R¹³, —(CH₂)ₙ—CO—NR¹⁵R¹⁶, —SR¹⁰ or —SO₂R¹⁸
or
R² and R³ together form a piperidino or morpholino ring,
A stands for the group 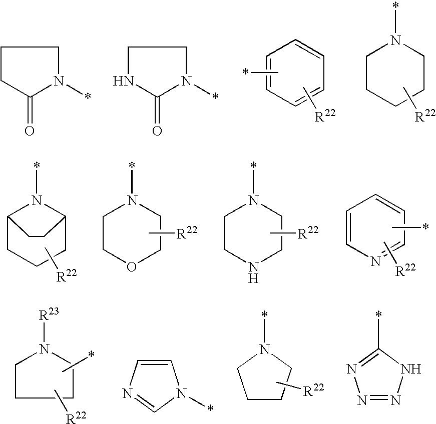
R⁴ stands for hydrogen, C₁-C₆-alkyl, halo-C₁-C₆-alkyl, hydroxy-C₁-C₆-alkyl, hydroxy-(CH₂)ₙ—O—(CH₂)ₙ—, or for the group —(CH₂)ₙ—CO—R²⁵, —(CH₂)ₙ—NR¹⁵R¹⁶, or for phenyl or benzyl that is optionally substituted with hydroxy-C₁-C₆-alkyl,
R⁸, R¹¹,
R¹² R¹⁴, R¹⁵
and R¹⁶ are the same or different and stand for hydrogen, C₁-C₁₀-alkyl, hydroxy-C₁-C₆-alkylene, (COOR¹⁴)—(CH₂)ₙ— or for phenyl, pyridyl, or pyrimidinyl that is optionally substituted with halogen or with the group —CO—C₁-C₆-alkyl, or for the group —COR¹³, —SO₂R¹⁸, —(CH₂)ₙ—NR¹⁵R¹⁶, —(CH₂)ₙ—C(CH₃)_{q}—(CH₂)ₙNR¹⁵R¹⁶ or 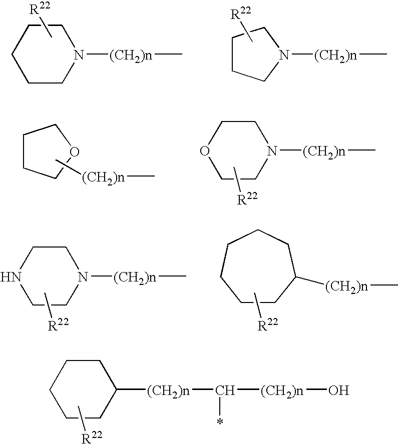
R¹⁰ stands for hydrogen, C₁-C₁₀-alkyl, hydroxy-C₁-C₆-alkylene, hydroxy-C₁-C₆-alkylenoxy-C₁-C₆-alkylene, C₁-C₆-alkoxy-CO—C₁-C₆-alkylene, —(CH₂)ₙ—CO—NR¹⁵R¹⁶ or for phenyl that is optionally substituted with halogen or with the group —CO—C—C₆-alkyl, or for the group —COR¹³—SO₂R¹⁸, or COOR¹⁴—(CH₂)ₙ—,
R¹³ stands for hydrogen, C₁-C₁₀-alkyl, C₁-C₁₀-alkenyl, C₁-C₁₀-alkinyl, C₁-C₆-alkyloxy-C₁-C₆-alkenyl, C₁-C₆-alkyloxy-C₁-C₆-alkenyloxy-C₁-C₆-alkenyl, phenyl or for the group 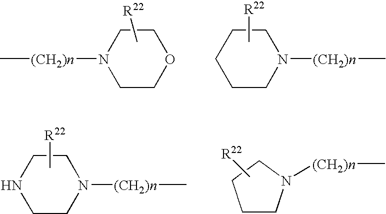
—R¹⁸ stands for C₁-C₁₀-alkyl, hydroxy, hydroxy-C₁-C₆-alkyl or for the group —NR¹¹R¹² 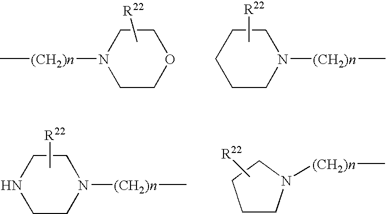
   for phenyl that is optionally substituted in one or more places in the same way or differently with C₁-C₆-alkyl,
R²² stands for hydrogen, hydroxy-C₁-C₆-alkyl, or for the group —OR¹⁰, —NR¹¹R¹², COR¹³, —CONR¹⁵R¹⁶, —SO₂R¹⁸, —NR¹⁵—(C═S)—NR¹⁶—(CH₂)ₙ—R²⁴, or —NR¹⁵—(C═O)—NR¹⁶—(CH₂)ₙ—R²⁴,
R²³ stands for hydrogen or C₁-C₆-alkyl,
R²⁴ stands for hydrogen, phenyl, C₁-C₆-alkoxy or for the group —(CH₂)ₙ—COO—C₁-C₆-alkyl,
R²⁵ stands for the group —OR¹⁰ or for C₂-C₆-alkenyl, phenyl, pyridyl, imidazolyl, morpholinyl, piperidinyl, C₃-C₆-cycloalkyl or 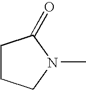
that is optionally substituted in one or more places in the same way or differently with halogen, C₁-C₆-alkyl, hydroxy-C₁-C₆-alkyl or with the group —OR¹⁰ or —COOR¹⁴,
m, p, k, in each case independently of one another, stand for 0 or 1,
n stands for 0, 1, 2, 3, 4, 5, 6, 7, 8, 9 or 10,
q stands for 1 or 2, as well as their stereoisomers, mixtures of the stereoisomers and their salts.

To use the compounds according to the invention as pharmaceutical agents, the latter are brought into the form of a pharmaceutical preparation, which in addition to the active ingredient for enteral or parenteral administration contains suitable pharmaceutical, organic or inorganic inert support media, such as, for example, water, gelatin, gum arabic, lactose, starch, magnesium stearate, talc, vegetable oils, polyalkylene glycols, etc. The pharmaceutical preparations can be present in solid form, for example as tablets, coated tablets, suppositories, or capsules, or in liquid form, for example as solutions, suspensions, or emulsions. Moreover, they optionally contain adjuvants, such as preservatives, stabilizers, wetting agents or emulsifiers; salts for changing the osmotic pressure or buffers.

These pharmaceutical preparations are also subjects of this invention.

For parenteral administration, especially injection solutions or suspensions, especially aqueous solutions of active compounds in polyhydroxyethoxylated castor oil, are suitable.

As carrier systems, surface-active adjuvants, such as salts of bile acids or animal or plant phospholipids, but also mixtures thereof, as well as liposomes or their components can also be used.

For oral administration, especially tablets, coated tablets or capsules with talc and/or hydrocarbon vehicles or binders, such as, for example, lactose, corn or potato starch, are suitable. The administration can also be carried out in liquid form, such as, for example, as a juice, to which optionally a sweetener is added.

Enteral, parenteral and oral administrations are also subjects of this invention.

The dosage of the active ingredients can vary depending on the method of administration, age and weight of the patient, type and severity of the disease to be treated and similar factors. The daily dose is 0.5-1000 mg, preferably 50-200 mg, whereby the dose can be given as a single dose to be administered once or divided into two or more daily doses.

Subjects of this invention also include the use of compounds of general formula I for the production of a pharmaceutical agent for treating cancer, auto-immune diseases, cardiovascular diseases, chemotherapy agent-induced alopecia and mucositis, infectious diseases, nephrological diseases, chronic and acute neurodegenerative diseases and viral infections, whereby cancer is defined as solid tumors and leukemia; auto-immune diseases are defined as psoriasis, alopecia and multiple sclerosis; cardiovascular diseases are defined as stenoses, arterioscleroses and restenoses; infectious diseases are defined as diseases that are caused by unicellular parasites; nephrological diseases are defined as glomerulonephritis; chronic neurodegenerative diseases are defined as Huntington's disease, amyotrophic lateral sclerosis, Parkinson's disease, AIDS dementia and Alzheimer's disease; acute neurodegenerative diseases are defined as ischemias of the brain and neurotraumas; and viral infections are defined as cytomegalic infections, herpes, hepatitis B or C, and HIV diseases.

Subjects of this invention also include pharmaceutical agents for treating the above-cited diseases, which contain at least one compound according to general formula I, as well as pharmaceutical agents with suitable formulation substances and vehicles.

The compounds of general formula I according to the invention are, i.a., excellent inhibitors of the polo-like kinases, such as Plk1, Plk2, Plk3, and Plk4.

If the production of the starting compounds is not described, the latter are known or can be produced analogously to known compounds or to processes that are described here. It is also possible to perform all reactions that are described here in parallel reactors or by means of combinatory operating procedures.

The isomer mixtures can be separated into the isomers, such as, e.g., into the enantiomers, diastereomers or E/Z isomers, according to commonly used methods, such as, for example, crystallization, chromatography or salt formation, if the isomers are not in a state of equilibrium with one another.

The production of the salts is carried out in the usual way by a solution of the compound of formula I being mixed with the equivalent amount of or excess base or acid, which optionally is in solution, and the precipitate being separated or the solution being worked up in the usual way.

Production of the Compounds According to the Invention

The following examples explain the production of the compounds according to the invention, without the scope of the claimed compounds being limited to these examples.

The compounds of general formula I according to the invention can be produced according to the following general diagrams of the process:

Diagram 1

Diagram 1

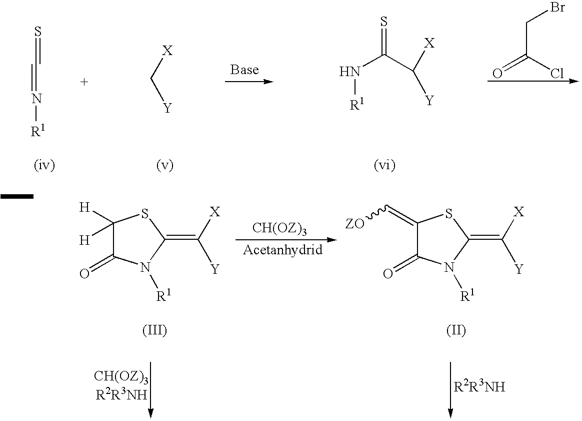

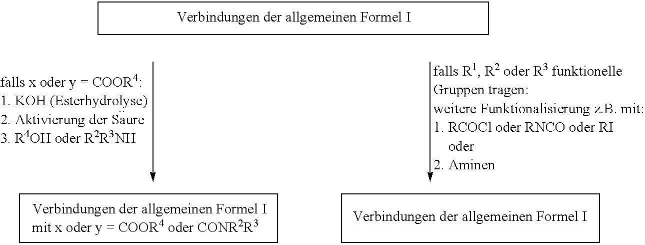

[Key to Diagram 1:]
Acetanhydrid=Acetic anhydride
Verbindungen der allgemeinen Formel I=Compounds of general formula I
falls x oder y=If x or y
Esterhydrolyse=ester hydrolysis
Aktivierung der Säure=activation of acid
oder=or
falls R¹, R² or R³ funktionelle Gruppen tragen=if R¹, R² or R³ carry functional groups weitere Funktionalisierung z.B. mit=Additional functionalization, e.g., with Aminen=Amines
Mit x oder y=with x or y

The production of the intermediate compounds of general formulas II and III, in which X, Y and R¹ have the meanings that are indicated in general formula I and Z stands for C₁-C₁₀-alkyl, is carried out from the educts of general formulas (Iv) to (vi), in which X, Y and R¹ have the meanings that are indicated in general formula I. First, compounds of general formula (v) are added to isothiocyanates of general formula (iv). The addition is usually performed in the presence of suitable bases. As bases, e.g., trialkylamines, but also sodium hydride or potassium hydride, are suitable.

By reaction of compounds of general formula (vi) with 1-halogen-substituted acyl halides or esters, the intermediate products of general formula III are then obtained. This reaction usually takes place in inert solvents, such as, e.g., tetrahydrofuran, at temperatures of between −20° C. and +50° C. The intermediate products of general formula II are obtained from the intermediate products of general formula III, e.g., by reaction with trialkylorthoformates and acetic acid anhydride in most cases at elevated temperature (100-200° C.).

From the compounds of general formula II, the compounds of general formula I according to the invention are produced by the addition of amines. This reaction can be carried out in all suitable organic solvents, such as, e.g., acetone, alcohols, dialkyl ethers, alkanes or cycloalkanes.

If the amines that are used are liquids, the reactions can also be performed without a solvent. The reaction temperatures in most cases are between −20° C. and +80° C. In addition to NH₃, the amines that are introduced can be primary or secondary.

Functional groups of educts and intermediate products can optionally be protected during the introduction.

The addition of amines to compounds of general formula II is accomplished under such reaction conditions that the use of parallel syntheses for the production of a large number of compounds of general formula I is easily possible.

As an alternative, the compounds of general formula I according to the invention can also be produced directly from the intermediate products of general formula III. In these cases, the amine is already added in the reaction with CH(OZ)₃, whereby Z has the meaning that is indicated in general formula II. These reactions are performed in most cases at temperatures of between 80-220° C.

All functional groups of general formulas I to III and iv to vi can be still further modified. Among the latter, e.g., the introduction of double and triple bonds, the hydrogenation of double and triple bonds, the introduction of additional substituents, the cleavage of esters, amides, ethers, etc., are defined. All protective groups that are introduced in the meantime are cleaved again in suitable intermediate or final stages.

Functional groups at substituents R¹, R² or R³ of general formula I, such as, for example, amines, alcohols, halides, or carboxylic acids, can be further functionalized especially to obtain additional compounds of general formula I.

If R² or R³ in the compounds of general formula I first stands for hydrogen, this radical optionally can take place with parallel syntheses by reaction with optionally substituted alkanoyl halides, arylalkanoyl halides, alkoxyalkanoyl halides, aryloxyalkanoyl, alkyl halides, isocyanates, isothiocyanates, or alkyl- or arylsulfonyl chlorides.

Subjects of this invention are thus also compounds of general formulas II and III,

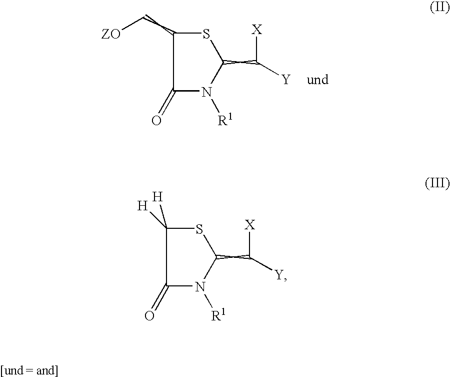

in which X, Y and R′ have the meanings that are indicated in general formula I, and Z stands for C₁-C₁₀-alkyl, as valuable intermediate products for the production of the compounds of general formula I according to the invention.

Preferred are those intermediate compounds of general formula II, in which Z stands for C₁-C₄ alkyl.

The examples below describe the production of the compounds according to the invention, without limiting the latter to the examples.

### EXAMPLE 1

### (E or Z)-Cyano-(3-ethyl-4-oxo-5-(E/Z)-phenylaminomethylene-thiazolidin-2-ylidene)-acetic acid ethyl ester

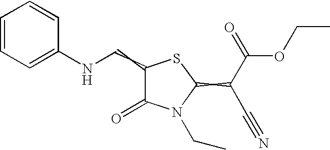 Process Variant A

3.4 g of the compound that is described under Example b) is suspended in 15 ml of ethylene glycol. 2.8 ml of triethyl orthoformate and 1.5 ml of aniline are added. The reaction mixture is refluxed for 2 hours in a water separator. Then, it is poured onto ice water. It is allowed to stir for 3 more hours, and then the precipitate is filtered off. The solid that is obtained is washed with water. Then, it is recrystallized from a mixture of ethyl acetate and diisopropyl ether. 2.9 g of product is obtained.

Process Variant B

A solution of 200 mg of the substance that is described under Example c) and 0.2 ml of aniline in 2 ml of acetone is stirred for 3 hours at 50° C. The product that is precipitated after cooling is filtered off and recrystallized from diisopropyl ether. 185 mg of product is obtained.

¹H-NMR (CDCl₃): δ=1.30-1.47 (6H); 4.30 (2H); 4.42 (2H); 7.04-7.18 (3H); 7.37 (2H); 7.62 (1H); 8.13 (1H, isomer B); 8.13 (1H, isomer B); 10.55 (1H) ppm.

### EXAMPLE 2

### 4-{[2-((E or Z)-Cyano-ethoxycarbonyl-methylene)-3-ethyl-4-oxo-thiazolidin-5-(E/Z)-ylidenemethyl]-amino}-benzoic acid ethyl ester

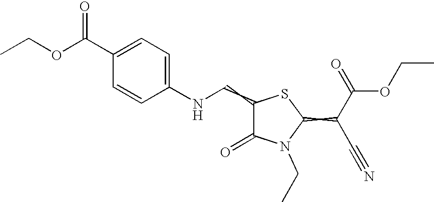

Analogously to Example 1, process variant A, 1.57 g of product is obtained from 2 g of the substance that is described under Example b), 1.7 ml of triethyl orthoformate and 1.65 g of 4-aminobenzoic acid ethyl ester.

¹H-NMR (D6-DMSO): δ=1.20-1.35 (9H); 4.20-4.35 (6H); 7.42 (2H); 7.49 (2H, isomer B); 7.90 (2H); 8.22 (1H); 8.51 (1H, isomer B); 10.70 (1H) ppm.

### EXAMPLE 3

### (E or Z)-Cyano-{3-ethyl-5-(E/Z)-[(4-methoxy-phenylamino)-methylene]-4-oxo-thiazolidin-2-ylidene}-acetic acid ethyl ester

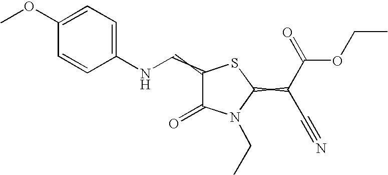

Analogously to Example 1, process variant A, 1.8 g of product is obtained from 2 g of the substance that is described under Example b), 1.7 ml of triethyl orthoformate and 1.23 g of 4-aminoanisole.

¹H-NMR (D6-DMSO): δ=1.22 (6H); 3.61 (3H); 4.22 (4H); 6.93 (2H); 7.28 (2H); 8.10 (1H); 8.38 (1H, isomer B); 10.49 (1H); 19.58 (1H, isomer B) ppm.

### EXAMPLE 4

### (E or Z)-(5-(E/Z)-{[Bis-(2-hydroxy-ethyl)-amino]-methylene}-3-ethyl-4-oxo-thiazolidin-2-ylidene)-cyanoacetic acid ethyl ester

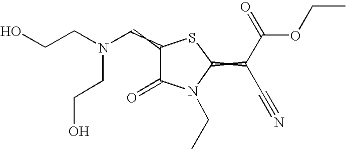

Analogously to Example 1, process variant B, 80 mg of product is obtained from 150 mg of the substance that is described under Example c), 0.05 ml of diethanolamine in 2 ml of acetone.

¹H-NMR (D6-DMSO): δ=1.15-1.28 (6H); 3.50-3.70 (8H); 4.15-4.30 (4H); 4.92 (1H); 5.09 (1H); 7.80 (1H) ppm.

### EXAMPLE 5

### (E or Z)-Cyano-(3-ethyl-4-oxo-5-(E/Z)-(piperidin-1-ylmethylene)-thiazolidin-2-ylidene)-acetic acid ethyl ester

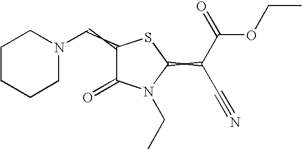

Analogously to Example 1, process variant B, 126 mg of product is obtained from 150 mg of the substance that is described under Example c), 0.056 ml of piperidine in 2 ml of acetone.

¹H-NMR (CDCl₃): δ=1.32 (6H); 1.72 (6H); 3.55 (4H); 4.29 (2H); 4.41 (2H); 7.65 (1H) ppm.

### EXAMPLE 6

### (E or Z)-Cyano-(3-ethyl-5-(E/Z)-(morpholin-4-ylmethylene)-4-oxo-thiazolidin-2-ylidene)-acetic acid ethyl ester

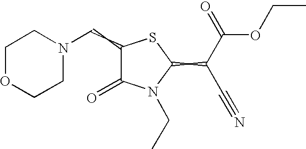

Analogously to Example 1, process variant B, 146 mg of product is obtained from 150 mg of the substance that is described under Example c), 0.05 ml of morpholine in 2 ml of acetone.

¹H-NMR (CDCl₃): δ=1.32 (6H); 3.60 (4H); 3.78 (4H); 4.29 (2H); 4.40 (2H); 7.60 (1H) ppm.

### EXAMPLE 7

### (E or Z)-Cyano-(5-(E/Z)-cyclohexylaminomethylene-3-ethyl-4-oxo-thiazolidin-2-ylidene)-acetic acid ethyl ester

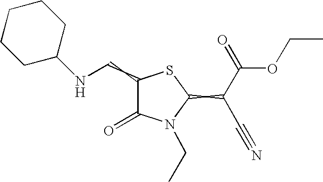

Analogously to Example 1, process variant B, 148 mg of product is obtained from 150 mg of the substance that is described under Example c), 0.065 ml of cyclohexylamine in 2 ml of acetone.

¹H-NMR (CDCl₃): δ=1.15-1.45 (12H); 1.78 (2H); 1.97 (2H); 3.25 (1H); 4.22-4.42 (4H); 5.00 (1H); 7.18 (1H, isomer B); 7.70 (1H); 8.82 (1H; isomer B) ppm.

### EXAMPLE 8

### (E or Z)-Cyano-(5-(E/Z)-diethylaminomethylene-3-ethyl-4-oxo-thiazolidin-2-ylidene)-acetic acid ethyl ester

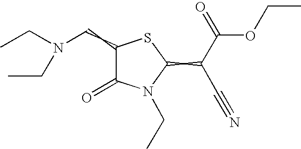

Analogously to Example 1, process variant B, 116 mg of product is obtained from 150 mg of the substance that is described under Example c), 0.058 ml of diethylamine in 2 ml of acetone.

¹H-NMR (D6-DMSO): δ=1.10-1.30 (12H); 3.50 (4H); 4.20 (4H); 7.80 (1H) ppm.

### EXAMPLE 9

### (E or Z)-Cyano-(3-ethyl-5-(E/Z)-{([(2-hydroxy-ethyl)-methyl-amino]-methylene}-4-oxo-thiazolidin-2-ylidene)-acetic acid ethyl ester

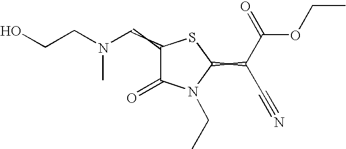

Analogously to Example 1, process variant B, 156 mg of product is obtained from 150 mg of the substance that is described under Example c), 0.045 ml of N-methylethanolamine in 2 ml of acetone.

¹H-NMR (D6-DMSO): δ=1.22 (6H); 3.27 (3H); 3.48-3.68 (4H); 4.20 (4H); 4.91 (1H); 7.78 (1H) ppm.

### EXAMPLE 10

### (E or Z)-{5-(E/Z)-[(4-Carbamoyl-phenylamino)-methylene]-3-ethyl-4-oxo-thiazolidin-2-ylidene}-cyanoacetic acid ethyl ester

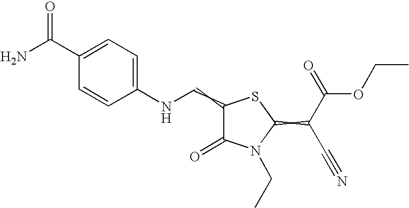

Analogously to Example 1, process variant B, 165 mg of product is obtained from 150 mg of the substance that is described under Example c), 76 mg of 4-aminobenzamide in 2 ml of acetone.

¹H-NMR (D6-DMSO): δ=1.23 (6H); 4.24 (4H); 7.26 (1H); 7.38 (2H); 7.45 (2H, isomer B); 7.89 (3H); 8.24 (1H); 8.51 (1H, isomer B); 10.65 (1H) ppm.

### EXAMPLE 11

### (E or Z)-(5-(E/Z)-Aminomethylene-3-ethyl-4-oxo-thiazolidin-2-ylidene)-cyanoacetic acid ethyl ester

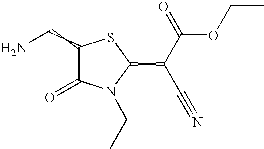

0.3 ml of a 2 molar ethanolic ammonia solution is added to a solution of 150 mg of the compound, described under Example c), in 2 ml of ethanol. It is allowed to stir for one more hour at 50° C. The product that is precipitated after cooling is filtered off and recrystallized from diisopropyl ether. 109 mg of product is obtained.

¹H-NMR (D6-DMSO): δ=1.13-1.28 (6H); 4.18 (4H); 7.70 (1H); 8.00-8.20 (2H) ppm.

### EXAMPLE 12

### (E or Z)-Cyano-(3-ethyl-4-oxo-5-(E/Z)-phenylaminomethylene-thiazolidin-2-ylidene)-acetic acid

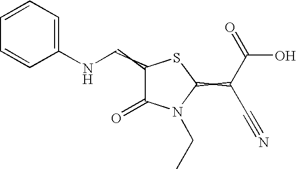

200 mg of the compound that is described under Example 1 is dissolved in 1 ml of dioxane. A solution of 200 mg of potassium hydroxide in 1 ml of ethanol is added, and it is stirred for 6 more hours at 70° C. Then, 1N HCl is added (pH 1). It is stirred for 2 more hours, and the precipitate is filtered off. The crude product is recrystallized from dichloromethane/methanol (8+2). 100 mg of product is obtained.

¹H-NMR (D6-DMSO): δ=1.21 (3H); 4.22 (2H); 7.08 (1H); 7.28-7.41 (4H); 8.17 (1H); 8.43 (1H, isomer B); 10.47 (1H); 10.52 (1H, isomer B) ppm.

### EXAMPLE 13

### 2-(3-Ethyl-4-oxo-5-(E/Z)-phenylaminomethylene-thiazolidin-2-ylidene)-malonic acid diethyl ester

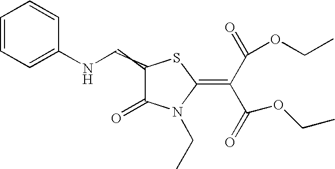

Analogously to Example 1, process variant A, 230 mg of product is obtained from 440 mg of the compound that is described under Example e), 0.4 ml of triethyl orthoformate and 0.2 ml of aniline in 5 ml of ethylene glycol.

¹H-NMR (CDCl₃): δ=1.15-1.38 (9H); 3.79 (2H); 4.25-4.38 (4H); 7.00-7.15 (3H); 7.42 (2H); 7.65 (1H); 10.46 (1H) ppm.

### EXAMPLE 14

### 2-(3-Ethyl-4-oxo-5-(E/Z)-phenylaminomethylene-thiazolidin-2-ylidene)-malononitrile

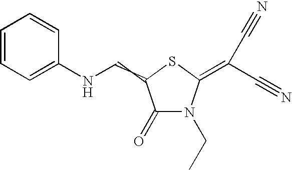

Analogously to Example 1, process variant B, 124 mg of product is obtained from 150 mg of the substance that is described under Example i), 0.06 ml of aniline in 2 ml of acetone.

¹H-NMR (D6-DMSO): δ=1.21 (3H); 4.10 (2H); 7.11 (1H); 7.30-7.43 (4H); 8.33 (1H); 10.58 (1H) ppm.

### EXAMPLE 15

### 2-(3-Ethyl-4-oxo-5-(E/Z)-[piperidin-1-ylmethylene]-thiazolidin-2-ylidene)-malononitrile

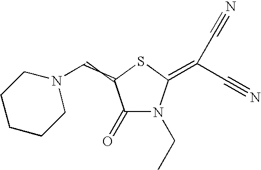

Analogously to Example 1, process variant B, 140 mg of product is obtained from 150 mg of the substance that is described under Example i), 0.066 ml of piperidine in 2 ml of acetone.

¹H-NMR (CDCl₃): δ=1.32 (3H); 1.72 (6H); 3.51 (4H); 4.21 (2H); 7.69 (1H) ppm.

### EXAMPLE 16

### 2-(3-Ethyl-5-(E/Z)-[morpholin-4-ylmethylene]-4-oxo-thiazolidin-2-ylidene)-malononitrile

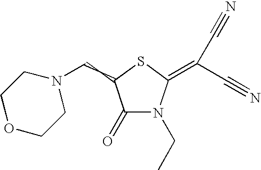

Analogously to Example 1, process variant B, 138 mg of product is obtained from 150 mg of the substance that is described under Example i), 0.058 ml of morpholine in 2 ml of acetone.

¹H-NMR (CDCl₃): δ=1.31 (3H); 3.56 (4H); 3.78 (4H); 4.23 (2H); 7.67 (1H) ppm.

### EXAMPLE 17

### 2-{3-Ethyl-5-(E/Z)-[(4-methoxy-phenylamino)-methylene]-4-oxo-thiazolidin-2-ylidene}-malononitrile

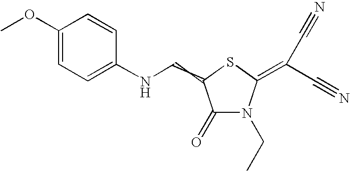

Analogously to Example 1, process variant B, 157 mg of product is obtained from 150 mg of the substance that is described under Example i), 82 mg of 4-aminoanisole in 2 ml of acetone.

¹H-NMR (D6-DMSO): δ=1.20 (3H, 3.72 (3H); 4.07 (2H); 6.94 (2H); 7.28 (2H); 8.23 (1H); 10.53 (1H) ppm.

### EXAMPLE 18

### 4-[(2-Dicyanomethylene-3-ethyl-4-oxo-thiazolidin-5-(E/Z)-ylidenemethyl)-amino]-benzamide

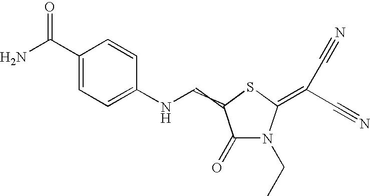

Analogously to Example 1, process variant B, 154 mg of product is obtained from 150 mg of the substance that is described under Example i), 90 mg of 4-aminobenzamide in 2 ml of ethanol.

¹H-NMR (D6-DMSO): δ=1.22 (3H); 4.08 (2H); 7.28 (1H); 7.38 (2H); 7.83-8.00 (3H); 8.40 (1H); 8.52 (1H, isomer B); 10.65 (1H) ppm.

### EXAMPLE 19

### 4-[(2-Dicyanomethylene-3-ethyl-4-oxo-thiazolidin-5-(E/Z)-ylidenemethyl)-amino]-benzoic acid ethyl ester

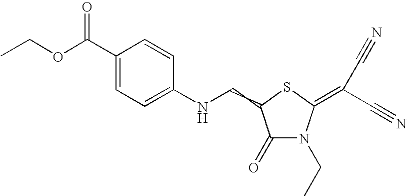

Analogously to Example 1, process variant B, 140 mg of product is obtained from 150 mg of the substance that is described under Example i), 110 mg of 4-aminobenzoic acid ethyl ester and 2 ml of acetone.

¹H-NMR (D6-DMSO): δ=1.38 (6H); 4.28 (2H); 4.37 (2H); 7.11 (2H); 7.14 (2H, isomer B); 7.69 (1H); 7.90 (1H, isomer B); 8.08 (2H); 8.25 (2H, isomer B); 10.57 ppm.

### EXAMPLE 20

### 2-(5-(E/Z)-Aminomethylene-3-ethyl-4-oxo-thiazolidin-2-ylidene)-malononitrile

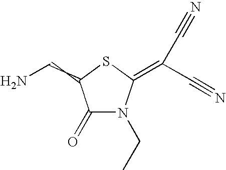

Analogously to Example 11, 101 mg of product is obtained from 150 mg of the compound that is described under Example i) and 0.3 ml of a 2 molar ethanolic ammonia solution in 2 ml of ethanol.

¹H-NMR (CDCl₃): δ=1.16 (3H); 4.02 (2H); 7.82 (1H); 8.10-8.40 (2H) ppm.

### EXAMPLE 21

### (E or Z)-(3-Ethyl-4-oxo-5-(E/Z)-phenylaminomethylene-thiazolidin-2-ylidene)-acetonitrile

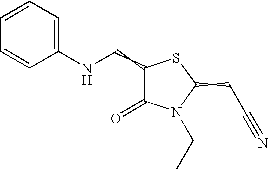

85 mg of the compound that is described under Example 12 is dissolved in 1 ml of methanol. 0.2 ml of 2N HCl is added and stirred for 30 minutes at 50° C. Then, it is poured onto ice water. The precipitate is suctioned off and recrystallized from methanol. 53 mg of product is obtained.

¹H-NMR (CDCl₃): δ=1.03 (3H); 3.70 (2H); 5.31 (1H); 7.03 (1H); 7.22 (2H); 7.31 (2H); 8.04 (1H); 9.76 (1H) ppm.

Analogously to Example 1, process variant B, the following compounds are produced from the intermediate product that is described under Example c):

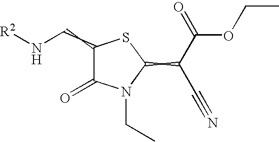

| Ex- | | | |
|---|---|---|---|
| ample | | Molecular | MS (ESI) |
| No. | R² | Weight | M + 1 |
| 22 | 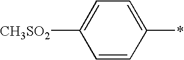 | 421.48 | 422 |
| 23 | 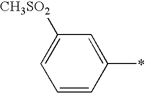 | 421.48 | 422 |
| 24 | 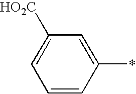 | 387.41 | 388 |
| 25 | 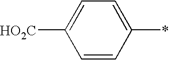 | 387.41 | 388 |
| 26 | 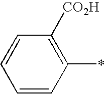 | 387.41 | 388 |
| 27 | 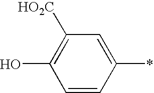 | 403.41 | 404 |
| 28 | 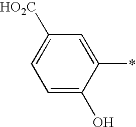 | 403.41 | 404 |
| 29 | 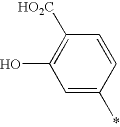 | 403.41 | 404 |
| 30 | 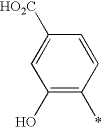 | 403.41 | 404 |
| 31 | 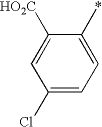 | 421.86 | 422/424 |
| 32 | 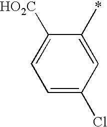 | 421.86 | 422/424 |
| 33 | 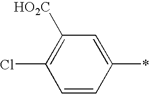 | 421.86 | 422/424 |
| 34 | 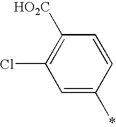 | 421.86 | 422/424 |
| 35 | 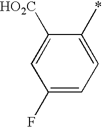 | 405.41 | 406 |
| 36 | 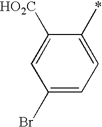 | 466.30 | 466/468 |
| 37 | 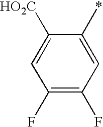 | 423.39 | 424 |
| 38 | 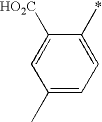 | 401.43 | 402 |
| 39 | 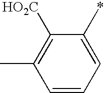 | 401.43 | 402 |
| 40 | 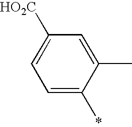 | 401.43 | 402 |
| 41 | 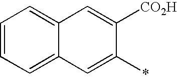 | 437.48 | 438 |
| 42 | 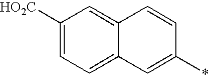 | 437.48 | 438 |
| 43 | 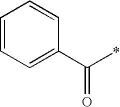 | 371.41 | 372 |
| 44 | 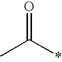 | 309.34 | 310 |
| 45 | 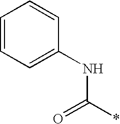 | 386.42 | 387 |
| 46 | 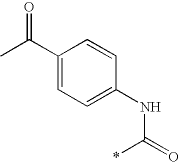 | 428.47 | 429 |
| 47 | 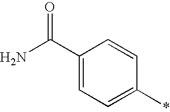 | 386.42 | 387 |
| 48 | 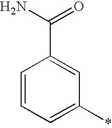 | 386.42 | 387 |
| 49 | 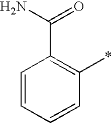 | 386.42 | 387 |
| 50 | 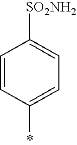 | 422.49 | 423 |
| 51 | 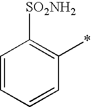 | 422.49 | 423 |
| 52 | 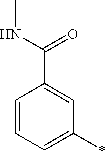 | 400.45 | 401 |
| 53 | 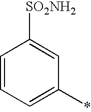 | 422.49 | 423 |
| 54 | 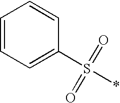 | 407.46 | 408 |
| 55 | 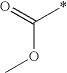 | 325.34 | 326 |
| 56 | 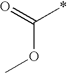 | 325.34 | 326 |
| 57 | 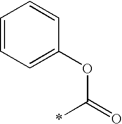 | 387.41 | 388 |
| 58 | 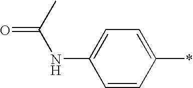 | 400.45 | 401 |
| 59 | 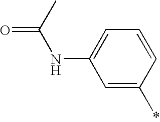 | 400.45 | 401 |
| 60 | 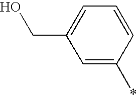 | 373.42 | 374 |
| 61 | 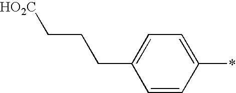 | 429.49 | 430 |

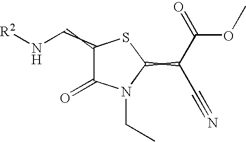

| Example | | | MS (ESI) |
|---|---|---|---|
| No. | R² | Molecular Weight | M + 1 |
| 62 | 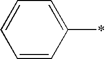 | 329.37 | 330 |

Analogously to Example 1, process variant B, the following compounds are produced from the intermediate product that is described under Example c):

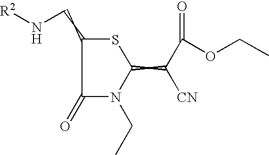

| Example | | | MS (ESI) |
|---|---|---|---|
| No. | R² | Molecular Weight | M + 1 |
| 63 | 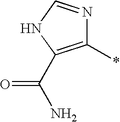 | 376.40 | 377 |
| 64 | 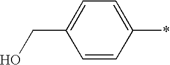 | 373.43 | 374 |
| 65 | 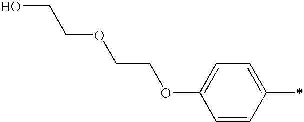 | 447.51 | 448 |
| 66 | 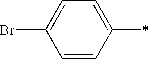 | 422.30 | 423 |
| 67 | 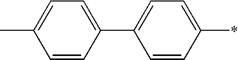 | 433.53 | 434 |
| 68 | 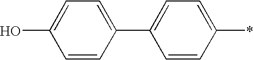 | 435.50 | 436 |
| 69 | 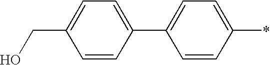 | 449.53 | 450 |
| 70 | 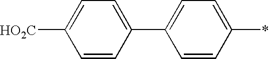 | 463.51 | 464 |

Analogously to Example 1, process variant B, the following compounds are produced from the intermediate product that is described under Example 1):

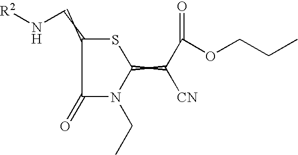

| Example | | Molecular | MS (ESI) |
|---|---|---|---|
| No. | R² | Weight | M + 1 |
| 71 | 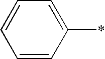 | 357.43 | 358 |
| 72 | 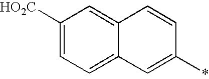 | 451.50 | 452 |
| 73 | 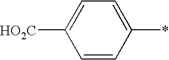 | 401.44 | 402 |
| 74 | 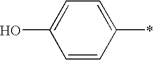 | 373.43 | 374 |
| 75 | 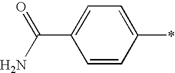 | 400.46 | 401 |
| 76 | 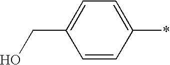 | 387.46 | 388 |

Analogously to Example 1, process variant B, the following compounds are produced from the intermediate product that is described under Example o):

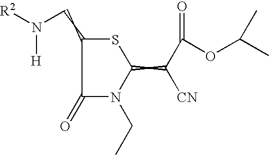

| Example | | Molecular | MS (ESI) |
|---|---|---|---|
| No. | R² | Weight | M + 1 |
| 77 | 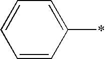 | 357.43 | 358 |
| 78 | 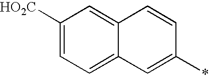 | 451.50 | 452 |
| 79 | 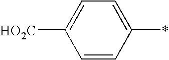 | 401.44 | 402 |
| 80 | 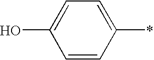 | 373.43 | 374 |
| 81 | 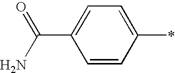 | 400.46 | 401 |
| 82 | 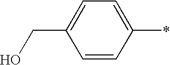 | 387.46 | 388 |

Analogously to Example 1, process variant B, the following compounds are produced from the intermediate product that is described under Example r):

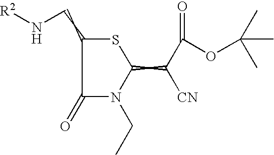

| Example | | Molecular | MS (ESI) |
|---|---|---|---|
| No. | R² | Weight | M + 1 |
| 83 | 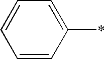 | 371.46 | 372 |
| 84 | 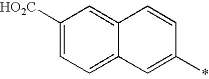 | 465.53 | 466 |
| 85 | 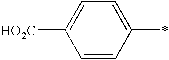 | 414.47 | 415 |
| 86 | 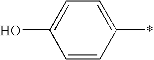 | 387.46 | 388 |
| 87 | 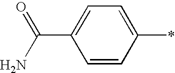 | 414.48 | 415 |

Analogously to Example 1, process variant B, the following compounds are produced from the intermediate product that is described under Example t):

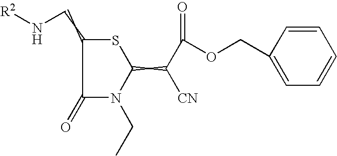

| Example | | Molecular | MS (ESI) |
|---|---|---|---|
| No. | R² | Weight | M + 1 |
| 88 | 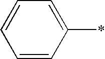 | 405.48 | 406 |
| 89 | 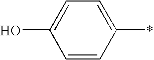 | 421.48 | 422 |
| 90 | 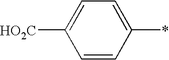 | 449.49 | 450 |
| 91 | 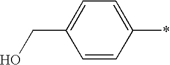 | 435.50 | 436 |
| 92 | 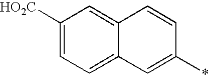 | 499.55 | 500 |
| 93 | 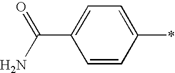 | 448.50 | 449 |
| 94 | 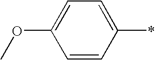 | 435.50 | 436 |

Analogously to Example 1, process variant B, the following compounds are produced from the intermediate product that is described under Example w):

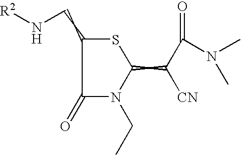

| Example | | Molecular | MS (ESI) |
|---|---|---|---|
| No. | R² | Weight | M + 1 |
| 95 | 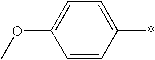 | 372.45 | 373 |
| 96 | 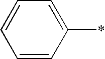 | 342.42 | 343 |
| 97 | 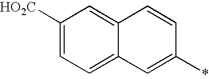 | 436.49 | 437 |
| 98 | 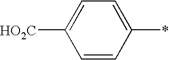 | 386.43 | 387 |
| 99 | 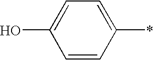 | 358.42 | 359 |
| 100 | 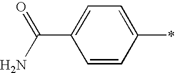 | 385.45 | 386 |
| 101 | 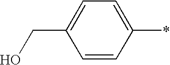 | 372.45 | 373 |

Analogously to Example 1, process variant B, the following compounds are produced from the intermediate product that is described under Example z):

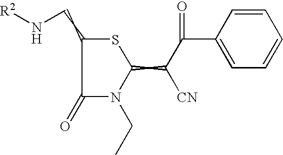

| Example | | | MS (ESI) |
|---|---|---|---|
| No. | R² | Molecular Weight | M + 1 |
| 102 | 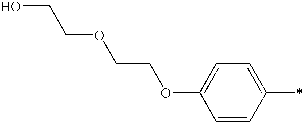 | 479.56 | 480 |
| 103 | 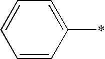 | 375.45 | 376 |
| 104 | 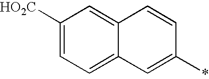 | 469.52 | 470 |
| 105 | 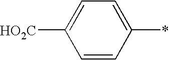 | 419.46 | 420 |
| 106 | 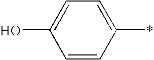 | 391.45 | 392 |
| 107 | 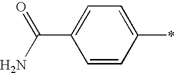 | 418.48 | 419 |
| 108 | 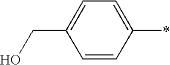 | 405.48 | 406 |
| 109 | 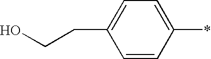 | 419.50 | 420 |
| 110 | 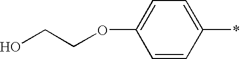 | 435.50 | 436 |
| 111 | 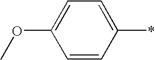 | 405.48 | 406 |

Analogously to Example 13, the following compounds are produced from the intermediate product that is described under Example e):

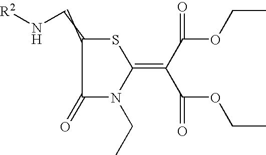

| Example | | Molecular | MS (ESI) |
|---|---|---|---|
| No. | R² | Weight | M + 1 |
| 112 | 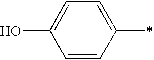 | 406.46 | 407 |
| 113 | 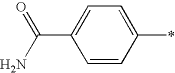 | 433.49 | 434 |
| 114 | 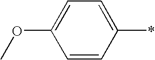 | 420.49 | 421 |
| 115 | 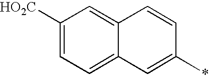 | 484.53 | 485 |

Analogously to Example 1, process variant A, the following compounds are produced from the intermediate product that is described under Example aa):

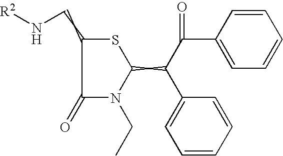

| Example | | Molecular | MS (ESI) |
|---|---|---|---|
| No. | R² | Weight | M + 1 |
| 116 | 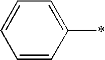 | 426.54 | 427 |
| 117 | 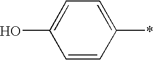 | 442.54 | 443 |

Analogously to Example 1, process variant A, the following compounds are produced from the intermediate product that is described under Example ab):

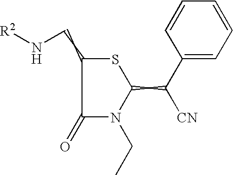

| Example | | Molecular | MS (ESI) |
|---|---|---|---|
| No. | R² | Weight | M + 1 |
| 118 | 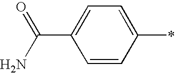 | 390.47 | 391 |
| 119 | 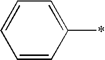 | 347.44 | 348 |
| 120 | 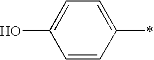 | 363.44 | 364 |
| 121 | 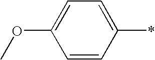 | 377.47 | 378 |
| 122 | 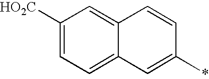 | 441.51 | 442 |

Analogously to Example 1, process variant B, the following compounds are produced from the intermediate product that is described under Example ag):

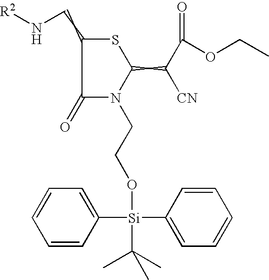

| Example | | Molecular | MS (ESI) |
|---|---|---|---|
| No. | R² | Weight | M + 1 |
| 123 | 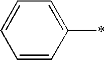 | 597.80 | 598 |
| 124 | 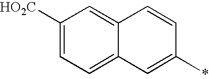 | 691.88 | 692 |
| 125 | 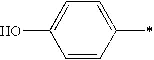 | 613.81 | 614 |
| 126 | 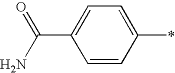 | 640.83 | 641 |
| 127 | 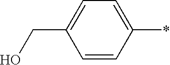 | 627.83 | 628 |
| 128 | 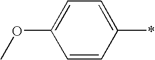 | 627.83 | 628 |

### EXAMPLE 129

### (E or Z)-Cyano-[3-(2-hydroxy-ethyl)-4-oxo-5-(E/Z)-phenylaminomethylene-thiazolidin-2-ylidene]-acetic acid ethyl ester

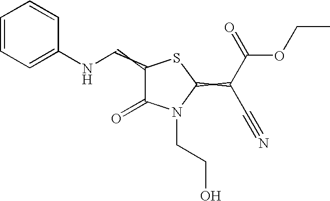

0.3 ml of a 1 molar solution of tetrabutylammonium fluoride in tetrahydrofuran is added to 125 mg of the compound, described under Example 123, in 5 ml of tetrahydrofuran. It is allowed to stir for 3 more hours at 50° C. Then, the reaction mixture is poured onto ice-cold saturated ammonium chloride solution. It is allowed to stir for 2 more hours and filtered. The crude product is recrystallized from a mixture that consists of ethanol and dichloromethane. 38 mg of product is obtained.

Molecular weight: 359.40; MS (ESI): [M+1]⁺-peak: 360.

Analogously to Example 129), the following Examples 130), 131), 132), 133) and 123) are produced from the compounds that are described under Examples 124), 125), 126), 127) and 128):

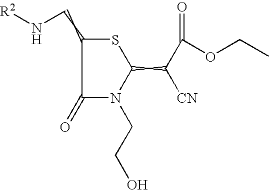

| Example | | Molecular | MS (ESI) |
|---|---|---|---|
| No. | R² | Weight | M + 1 |
| 130 | 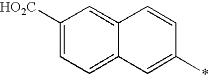 | 453.47 | 454 |
| 131 | 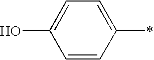 | 375.40 | 376 |
| 132 | 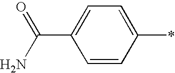 | 402.43 | 403 |
| 133 | 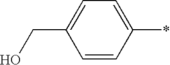 | 389.43 | 390 |
| 134 | 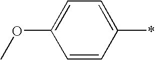 | 389.43 | 390 |

### EXAMPLE 135

### (E or Z)-{5-(E/Z)-[3-(2-Chloro-phenyl)-ureidomethylene]-3-ethyl-4-oxo-thiazolidin-2-ylidene}-cyanoacetic acid ethyl ester

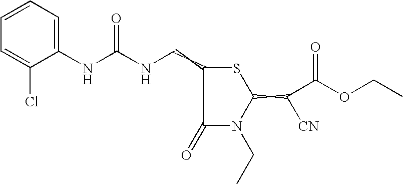

135 μl of 2-chlorophenyl isocyanate is added to a solution that consists of 150 mg of the compound, described under Example 11, in 5 ml of tetrahydrofuran. It is heated in a bomb tube for 48 hours to 100° C. After cooling, the reaction mixture is concentrated by evaporation in a vacuum. The residue is purified by column chromatography on silica gel with a mixture that consists of hexane/ethyl acetate. 181 mg of product is obtained.

1H-NMR (DMSO-d6), main isomer: δ=1.30-1.42 (6H); 4.18-4.30 (4H); 7.12 (1H); 7.35 (1H); 7.51 (1H); 8.00 (1H); 8.25 (1H); 8.78 (1H); 11.08 (1H) ppm.

Analogously to Example 135), the following compounds are produced:

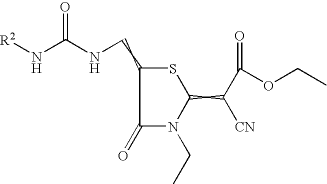

| Example | | Molecular | MS (ESI) |
|---|---|---|---|
| No. | R² | Weight | M + 1 |
| 136 | 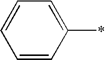 | 386.43 | 387 |
| 137 | 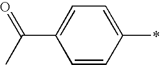 | 428.47 | 429 |

### EXAMPLE 138

### (E or Z)-Cyano-{3-ethyl-4-oxo-5-(E/Z)-[(toluene-4-sulfonylamino)-methylene]-thiazolin-2-ylidene}-acetic acid ethyl ester

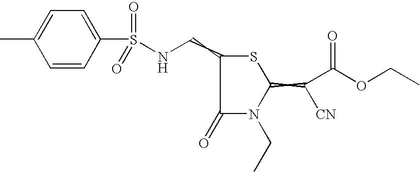

233 μl of triethylamine and 161 mg of p-toluenesulfonic acid chloride are added to a solution that consists of 150 mg of the compound, described under Example 11, in 5 ml of tetrahydrofuran. It is refluxed for 48 hours. Then, the reaction mixture is poured onto ice-cold 2N hydrochloric acid. It is extracted with ethyl acetate, the organic phase is washed with saturated sodium chloride solution, dried on sodium sulfate and concentrated by evaporation in a vacuum. The residue is purified by column chromatography on silica gel with a mixture that consists of hexane/ethyl acetate. 155 mg of product is obtained.

1H-NMR (DMSO-d6): δ=1.12-1.24 (6H); 2.33 (3H); 4.15-4.22 (4H); 7.31 (2H); 7.62 (2H); 8.18 (1H) ppm.

### EXAMPLE 139

### (E or Z)-[5-(E/Z)-(Benzenesulfonylamino-methylene)-3-ethyl-4-oxo-thiazolidin-2-ylidene]-cyanoacetic acid ethyl ester

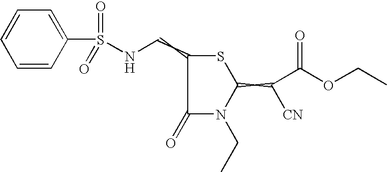

Example 139 is produced analogously to the compound that is described under Example 138).

1H-NMR (DMSO-d6): δ=1.12-1.25 (6H); 4.10-4.22 (4H); 7.52-7.67 (3H); 7.78 (2H); 8.05 (1H) ppm.

### EXAMPLE 140

### (E or Z)-Cyano-[5-(E/Z)—(N,N-dimethylaminosulfonylamino-methylene)-3-ethyl-4-oxo-thiazolidin-2-ylidene]-acetic acid ethyl ester

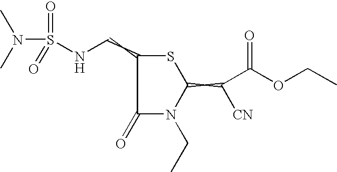

470 μl of triethylamine and 180 μl of N,N-dimethylamidosulfonic acid chloride are added to a solution that consists of 150 mg of the compound, described under Example 11, in 5 ml of toluene. It is refluxed for 16 hours. Then, the reaction mixture is poured onto ice-cold 2N hydrochloric acid. It is extracted with ethyl acetate, the organic phase is washed with saturated sodium chloride solution, dried on sodium sulfate and concentrated by evaporation in a vacuum. The residue is purified by column chromatography on silica gel with a mixture that consists of hexane/ethyl acetate. 52 mg of product is obtained.

1H-NMR (DMSO-d6): δ=1.12-1.22 (6H); 2.60 (6H); 4.10-4.25 (4H); 8.05 (1H) ppm.

### EXAMPLE 141

### (E or Z)-Cyano-[3-(2-methoxy-ethyl)-4-oxo-5-(E/Z)-phenylaminomethylene-thiazolidin-2-ylidene]-acetic acid ethyl ester

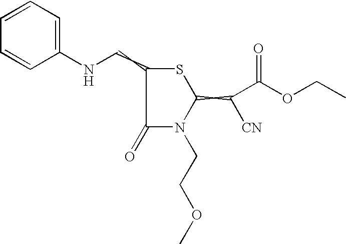

Analogously to Example 1, process variant B, 123 mg of product is obtained from 150 mg of the compound that is described under Example aj) and 4611 of aniline in 3 ml of ethanol.

1H-NMR (DMSO-d6), main isomer: δ=1.23 (3H); 3.25 (3H); 3.61 (2H); 4.20 (2H); 4.46 (2H); 7.11 (1H); 7.30-7.43 (5H); 8.20 (1H) ppm

Analogously to Example 1, process variant B, the following compounds are produced from the intermediate product that is described under Example am):

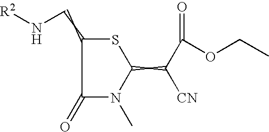

| Example | | Molecular | MS (ESI) |
|---|---|---|---|
| No. | R² | Weight | M + 1 |
| 142 | 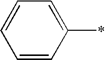 | 329.38 | 330 |
| 143 | 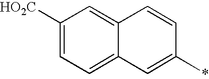 | 423.45 | 424 |
| 144 | 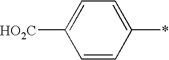 | 373.39 | 374 |
| 145 | 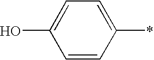 | 345.38 | 346 |
| 146 | 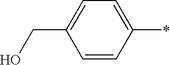 | 359.40 | 360 |
| 147 | 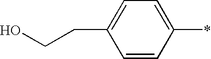 | 373.43 | 374 |
| 148 | 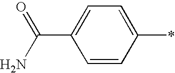 | 372.40 | 373 |
| 149 | 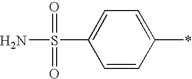 | 408.46 | 409 |

### EXAMPLE 150

### (E or Z)-Cyano-(3-cyclobutyl-4-oxo-5-(E/Z)-phenylaminomethylene-thiazolidin-2-ylidene)-acetic acid ethyl ester

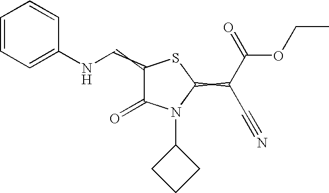

50 mg of the compound that is described under Example aq) and 17 mg of aniline are introduced into 2 ml of ethanol and stirred under reflux for 3 hours. The product that is precipitated after the cooling is filtered off and recrystallized twice from ethanol. 12 mg of the title compound is obtained as a pH-dependent 5-(E/Z)-isomer mixture.

1H-NMR (DMSO-d6, stored with K₂CO₃, main isomer): δ=1.25 (3H); 1.40-1.90 (2H); 2.35 (2H); 2.90 (2H); 4.23 (2H); 5.13 (1H); 7.10 (1H); 7.25-7.43 (4H); 8.15 (1H); 10.45 (1H) ppm.

Analogously to the compound that is described under Example 150), the following compounds are also produced:

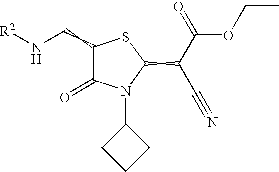

| Example | | Molecular | MS (ESI) | Synthesis |
|---|---|---|---|---|
| No. | R² | Weight | [M + 1]⁺ | Analogous to |
| 151 | 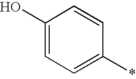 | 385.44 | 386 | Example No. 150 |
| 152 | 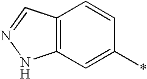 | 409.47 | 410 | Example No. 150 |
| 153 | 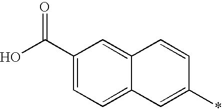 | 463.51 | 464 | Example No. 150 |
| 154 | 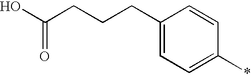 | 455.53 | 456 | Example No. 150 |
| 155 | 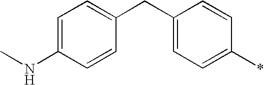 | 488.61 | 489 | Example No. 150 |
| 156 | 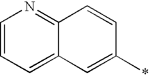 | 420.49 | 421 | Example No. 150 |
| 157 | 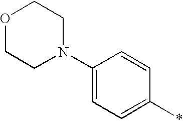 | 454.55 | 455 | Example No. 150 |
| 158 | 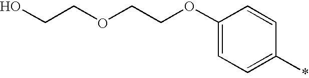 | 473.55 | 474 | Example No. 150 |

### EXAMPLE 159

### (E or Z)-Cyano-{3-ethyl-4-oxo-5-(E/Z)-[(4-sulfo-phenylamino)-methylene]-thiazolidin-2-ylidene}-acetic acid ethyl ester

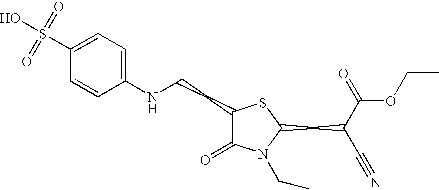

100 mg of the compound that is described under Example c), 0.1 ml of triethylamine and 74 mg of 4-sulfanilic acid are introduced into 2 ml of ethanol and stirred under reflux for 3 hours. The solvent is removed, and the crude product is recrystallized from ethanol. After treatment with acidic ion exchanger, 40 mg of the title compound is obtained as a pH-dependent 5-(E/Z)-isomer mixture.

1H-NMR (DMSO-d6, stored with K₂CO₃, main isomer): δ=1.10-1.45 (6H); 4.15-4.35 (4H); 7.27 (2H); 7.57 (2H); 8.21 (1H); 10.60 (1H) ppm.

### EXAMPLE 160

### (E or Z)-Cyano-{3-ethyl-5-(E/Z)-[(6-hydroxy-naphthalen-1-ylamino)-methylene]-4-oxo-thiazolidin-2-ylidene}-acetic acid ethyl ester

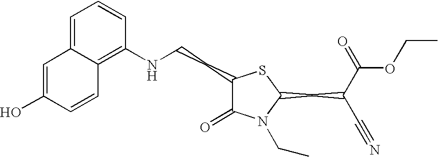

100 mg of the compound that is described under Example c) and 68 mg of 1-amino-6-hydroxynaphthalene are introduced into 2 ml of ethanol and stirred for 3 hours under reflux. The solvent is removed, and the crude product is recrystallized from ethanol. 82 mg of the title compound is obtained as a pH-dependent 5-(E/Z)-isomer mixture.

1H-NMR (DMSO-d6, stored with K₂CO₃): δ=1.15-1.35 (6H); 4.10-4.30 (4H); 7.08-7.22 (3H); 7.40 (1H); 7.60 (1H); 8.01 (1H, isomer A); 8.08 (1H); 8.70 (1H, isomer B); 9.95 (1H, isomer A); 10.01 (1H, isomer B); 10.65 (1H, isomer A); 11.40 (1H, isomer B) ppm.

Similarly produced are also the following compounds:

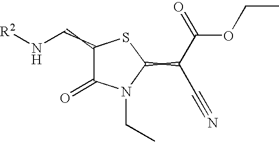

| Example | | Molecular | MS (ESI) | Synthesis |
|---|---|---|---|---|
| No. | R² | Weight | [M + 1]⁺ | Analogous to |
| 161 | 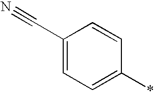 | 368.42 | 369 | Example No. 160 |
| 162 | 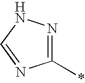 | 334.36 | 335 | Example No. 160 |
| 163 | 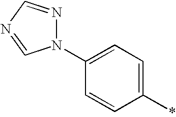 | 410.46 | 411 | Example No. 160 |
| 164 | 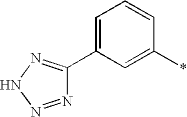 | 411.44 | 412 | Example No. 160 |
| 165 | 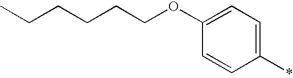 | 443.57 | 444 | Example No. 160 |
| 166 | 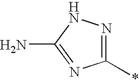 | 349.37 | 350 | Example No. 160 |
| 167 | 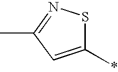 | 364.45 | 365 | Example No. 160 |
| 168 | 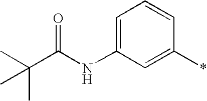 | 442.54 | 443 | Example No. 160 |
| 169 | 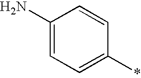 | 358.42 | 359 | Example No. 160 |
| 170 | 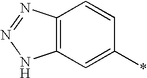 | 384.418 | 385 | Example No. 160 |

### EXAMPLE 171

### (E or Z)-Cyano-{3-ethyl-4-oxo-5-(E/Z)-[(3-piperidin-1-ylmethyl-phenylamino)-methylene]-thiazolidin-2-ylidene}-acetic acid ethyl ester

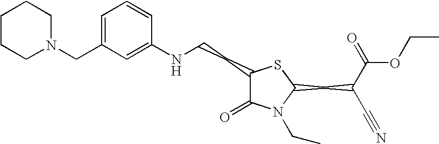

60 mg of the compound that is described under Example ar), 110 mg of potassium carbonate and 18 μl of piperidine are dissolved in 2 ml of DMF and stirred for 24 hours at room temperature. The reaction mixture is mixed with dichloromethane and washed three times with water. After purification by chromatography on silica gel, 22 mg of the title compound is obtained as a pH-dependent 5-(E/Z)-isomer mixture.

1H-NMR (DMSO-d6, stored with K₂CO₃, main isomer): δ=1.13-1.34 (6H); 1.34-1.57 (6H); 2.20-2.37 (4H); 3.40 (2H); 4.15-4.33 (4H); 7.00 (1H); 7.12-7.34 (3H); 8.20 (1H); 10.56 (1H) ppm.

Similarly produced are also the following compounds:

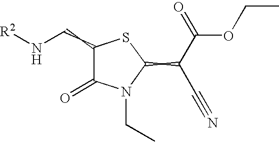

| Example | | Molecular | MS (ESI) | Synthesis |
|---|---|---|---|---|
| No. | R² | Weight | [M + 1]⁺ | Analogous to |
| 172 | 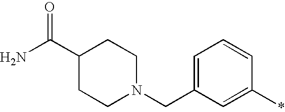 | 483.59 | 484 | Example No. 171 |
| 173 | 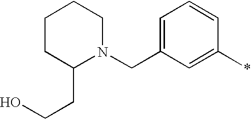 | 484.62 | 485 | Example No. 171 |
| 174 | 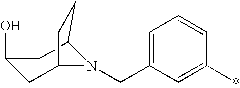 | 482.6 | 483 | Example No. 171 |
| 175 | 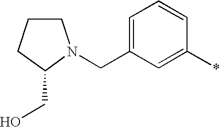 | 456.56 | 457 | Example No. 171 |
| 176 | 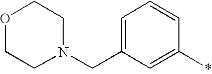 | 442.54 | 443 | Example No. 171 |
| 177 | 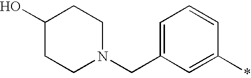 | 456.56 | 457 | Example No. 171 |

### EXAMPLE 178

### (E or Z)-Cyano-(3-ethyl-5-(E/Z)-{[4-(2-morpholin-4-yl-ethoxy)-phenylamino]-methylene}-4-oxo-thiazolidin-2-ylidene)-acetic acid ethyl ester

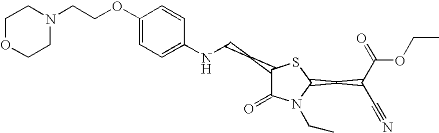

84 mg of the compound that is described under Example av), 97 mg of potassium carbonate and 18 μl of morpholine are dissolved in 5 ml of DMF and stirred for 18 hours at room temperature. The solvent is condensed under high vacuum, the residue is taken up in ethyl acetate and washed three times with water. After purification by chromatography on silica gel, 23 mg of the title compound is obtained as a pH-dependent 5-(E/Z)-isomer mixture.

1H-NMR (DMSO-d6, stored with K₂CO₃, main isomer): δ=1.15-1.30 (6H); 2.38-2.55 (4H); 2.68 (2H); 3.54 (4H); 4.05 (2H); 4.15-4.30 (4H); 6.94 (2H); 7.20 (2H); 8.14 (1H); 10.48 (1H) ppm.

Similarly produced are also the following compounds:

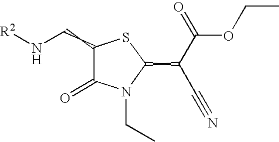

| Example | | Molecular | MS (ESI) | Synthesis |
|---|---|---|---|---|
| No. | R² | Weight | [M + 1]⁺ | Analogous to |
| 179 | 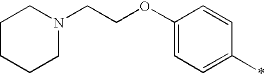 | 470.6 | 471 | Example No. 178 |
| 180 | 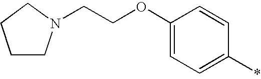 | 456.6 | 457 | Example No. 178 |
| 181 | 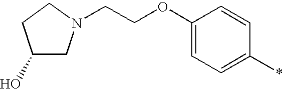 | 472.6 | 473 | Example No. 178 |
| 182 | 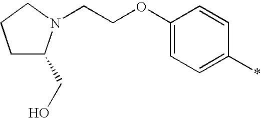 | 486.6 | 487 | Example No. 178 |
| 183 | 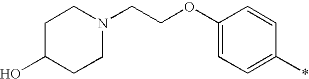 | 486.6 | 487 | Example No. 178 |
| 184 | 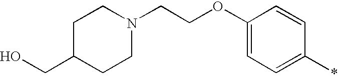 | 500.6 | 501 | Example No. 178 |
| 185 | 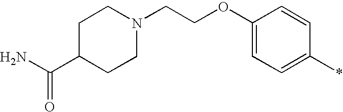 | 513.6 | 514 | Example No. 178 |
| 186 | 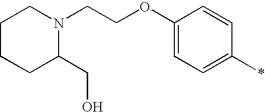 | 500.6 | 501 | Example No. 178 |
| 187 | 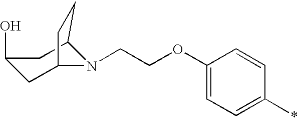 | 512.6 | 513 | Example No. 178 |
| 188 | 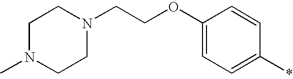 | 485.6 | 486 | Example No. 178 |

### EXAMPLE 189

### (E or Z)-(5-(E/Z)-{[3-(4-Acetyl-piperazin-1-ylmethyl)-phenylamino]-methylene}-3-ethyl-4-oxo-thiazolidin-2-ylidene)-cyanoacetic acid ethyl ester

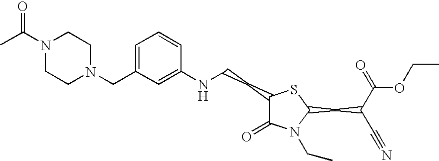

60 mg of the compound that is described under Example at) is dissolved in 2 ml of THF, mixed with 4111 of triethylamine and 8.5 μl of acetyl chloride, and stirred for 2 hours at room temperature. The reaction mixture is mixed with water and extracted with ethyl acetate. After purification by chromatography on silica gel, 19 mg of the title compound is obtained as a pH-dependent 5-(E/Z)-isomer mixture.

1H-NMR (DMSO-d6, stored with K₂CO₃, main isomer): δ=1.11-1.35 (6H); 1.18 (3H); 2.22-2.42 (4H); 3.38-3.55 (6H); 4.13-4.31 (4H); 7.03 (1H); 7.15-7.38 (3H); 8.20 (1H); 10.57 (1H) ppm.

### EXAMPLE 190

### (E or Z)-[5-(E/Z)-({Acetyl-[3-(4-acetyl-piperazin-1-ylmethyl)-phenyl]-amino}-methylene)-3-ethyl-4-oxo-thiazolidin-2-ylidene]-cyanoacetic acid ethyl ester

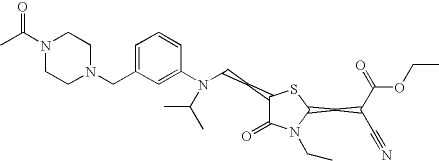

60 mg of the compound that is described under Example at) is dissolved in 2 ml of THF, mixed with 45 μl of triethylamine and 16 μl of acetyl chloride and stirred overnight at room temperature. The reaction mixture is mixed with water and extracted with ethyl acetate. After purification by chromatography on silica gel, 42 mg of the title compound is obtained as a pH-dependent 5-(E/Z)-isomer mixture.

1H-NMR (DMSO-d6, stored with K₂CO₃, main isomer): δ=1.10-1.30 (6H); 1.95 (3H); 2.02 (3H); 2.26-2.47 (4H); 3.25-3.40 (4H); 3.55 (2H); 4.01-4.25 (4H); 7.37-7.49 (2H); 7.51-7.68 (2H); 8.58 (1H) ppm.

### EXAMPLE 191

### (E or Z)-Cyano-(3-ethyl-5-(E/Z)-{[3-(4-methanesulfonyl-piperazin-1-ylmethyl)-phenylamino]-methylene}-4-oxo-thiazolidin-2-ylidene)-acetic acid ethyl ester

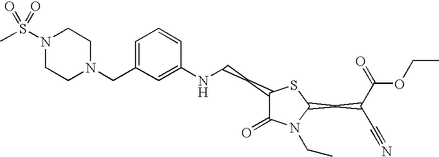

Analogously to Example 189), after purification by chromatography on silica gel, 35 mg of the title compound is obtained as a pH-dependent 5-(E/Z)-isomer mixture from 60 mg of the compound that is described under Example at), 4511 of triethylamine and 16 mg of methanesulfonic acid chloride.

1H-NMR (DMSO-d6, stored with K₂CO₃, main isomer): δ=1.12-1.34 (6H); 2.38-2.56 (4H); 2.88 (3H); 3.04-3.18 (4H); 3.51 (2H); 4.14-4.32 (4H); 7.05 (1H); 7.18-7.38 (3H); 8.20 (1H); 10.56 (1H) ppm.

### EXAMPLE 192

### (E or Z)-(5-(E/Z)-{[3-(4-tert-Butylcarbamoyl-piperazin-1-ylmethyl)-phenylamino]-methylene}-3-ethyl-4-oxo-thiazolidin-2-ylidene)-cyano-acetic acid ethyl ester

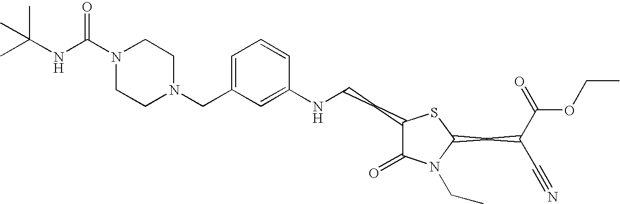

Analogously to Example 189), after purification by chromatography on silica gel, 31 mg of the title compound is obtained as a pH-dependent 5-(E/Z)-isomer mixture from 60 mg of the compound that is described under Example at), 45 μl of triethylamine and 14 mg of tert-butyl isocyanate.

1H-NMR (DMSO-d6, stored with K₂CO₃, main isomer): δ=1.14-1.35 (15H); 2.20-2.35 (4H); 3.15-3.28 (4H); 3.46 (2H); 4.15-4.33 (4H); 5.68-5.79 (1H); 7.03 (1H); 7.15-7.38 (3H); 8.21 (1H); 10.57 (1H) ppm.

### EXAMPLE 193

### (E or Z)-Cyano-(5-(E/Z)-{[3-(4-dimethylsulfamoyl-piperazin-1-ylmethyl)-phenylamino]-methylene}-3-ethyl-4-oxo-thiazolidin-2-ylidene)-acetic acid ethyl ester

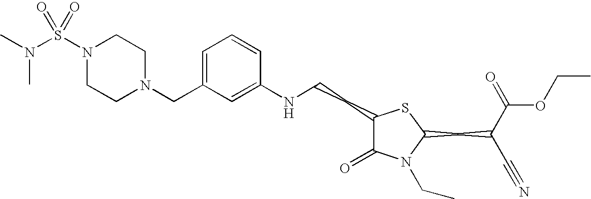

Analogously to Example 189), after purification by chromatography on silica gel, 15 mg of the title compound is obtained as a pH-dependent 5-(E/Z)-isomer mixture from 60 mg of the compound that is described under Example at), 45 μl of triethylamine and 20 mg of N,N-dimethylamidosulfonic acid chloride.

1H-NMR (DMSO-d6, stored with K₂CO₃, main isomer): δ=1.15-1.35 (6H); 2.35-2.50 (4H); 2.75 (6H); 3.16 (4H); 3.51 (2H); 4.15-4.32 (4H); 7.02 (1H); 7.14-7.37 (3H); 8.22 (1H); 10.59 (1H) ppm.

Similarly produced are also the following compounds:

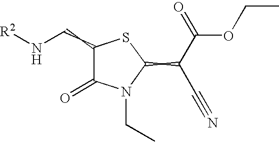

| Example | | Molecular | MS (ESI) | Synthesis |
|---|---|---|---|---|
| No. | R² | Weight | [M + 1]⁺ | Analogous to |
| 194 | 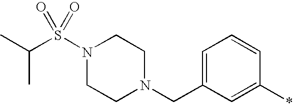 | 547.7 | 548 | Example No. 191 |
| 195 | 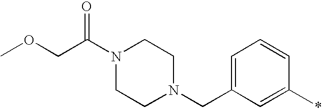 | 513.6 | 514 | Example No. 189 |
| 196 | 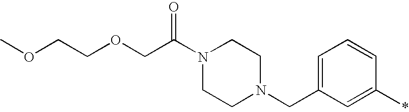 | 557.7 | 558 | Example No. 189 |

### EXAMPLE 197

### (E or Z)-Cyano-(3-ethyl-5-(E/Z)-{[3-(morpholine-4-carbonyl)-phenylamino]-methylene}-4-oxo-thiazolidin-2-ylidene)-acetic acid ethyl ester

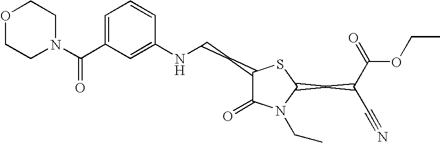

100 mg of the compound that is described under Example 24), 0.04 ml of triethylamine and 93 mg of TBTU are introduced into 2 ml of DMF and stirred for 30 minutes at room temperature. 26 μl of morpholine is added, and it is stirred overnight at room temperature. The reaction mixture is mixed with sodium bicarbonate solution and extracted with ethyl acetate. After purification by chromatography on silica gel, 57 mg of the title compound is obtained as a pH-dependent 5-(E/Z)-isomer mixture.

1H-NMR (DMSO-d6, stored with K₂CO₃, main isomer): δ=1.18-1.32 (6H); 3.45-3.75 (8H); 4.15-4.30 (4H); 7.10 (1H); 7.30-7.48 (3H); 8.25 (1H); 10.57 (1H) ppm.

### EXAMPLE 198

### (E or Z)-Cyano-(3-ethyl-5-(E/Z)-{[3-(2-morpholin-4-yl-ethylcarbamoyl)-phenylamino]-methylene}-4-oxo-thiazolidin-2-ylidene)-acetic acid ethyl ester

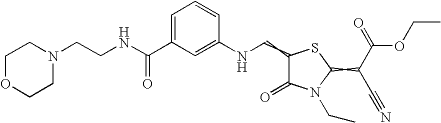

Analogously to Example 197), after purification by chromatography on silica gel, 26 mg of the title compound is obtained as a pH-dependent 5-(E/Z)-isomer mixture from 100 mg of the compound that is described under Example 24), 0.04 ml of triethylamine, 93 mg of TBTU and 39 μl of 4-(2-aminoethyl)morpholine.

1H-NMR (DMSO-d6, stored with K₂CO₃, main isomer): δ=1.18-1.35 (6H); 2.35-2.50 (6H); 3.40 (2H); 3.58 (4H); 4.15-4.35 (4H); 7.45 (2H); 7.57 (1H); 7.77 (1H); 8.30 (1H); 8.53 (1H); 10.65 (1H) ppm.

### EXAMPLE 199

### (E or Z)-Cyano-(3-ethyl-5-(E/Z)-{[4-(2-morpholin-4-yl-ethylcarbamoyl)-phenylamino]-methylene}-4-oxo-thiazolidin-2-ylidene)-acetic acid ethyl ester

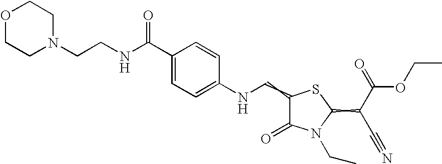

Analogously to Example 197), after purification by chromatography on silica gel, 84 mg of the title compound is obtained as a pH-dependent 5-(E/Z)-isomer mixture from 100 mg of the compound that is described under Example 25), 0.04 ml of triethylamine, 93 mg of TBTU and 39 μl of 4-(2-aminoethyl)morpholine.

1H-NMR (DMSO-d6, stored with K₂CO₃, main isomer): δ=1.15-1.34 (6H); 2.34-2.48 (6H); 3.30-3.45 (2H); 3.50-3.64 (4H); 4.15-4.33 (4H); 7.33 (2H); 7.82 (2H); 8.21-8.40 (2H); 10.65 (1H) ppm.

### EXAMPLE 200

### (E or Z)-Cyano-(3-ethyl-5-(E/Z)-{[4-(morpholine-4-carbonyl)-phenylamino]-methylene}-4-oxo-thiazolidin-2-ylidene)-acetic acid ethyl ester

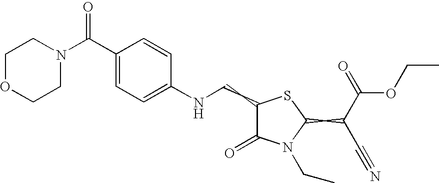

Analogously to Example 197), after purification by chromatography on silica gel, 40 mg of the title compound is obtained as a pH-dependent 5-(E/Z)-isomer mixture from 100 mg of the compound that is described under Example 25), 0.04 ml of triethylamine, 93 mg of TBTU and 26 μl of morpholine.

1H-NMR (DMSO-d6, stored with K₂CO₃, main isomer): δ=1.15-1.35 (6H); 3.40-3.70 (8H); 4.16-4.32 (4H); 7.27-7.48 (4H); 8.25 (1H); 10.64 (1H) ppm.

Similarly produced are also the following compounds:

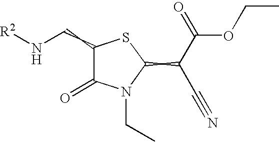

| Example | | Molecular | MS (ESI) | Synthesis |
|---|---|---|---|---|
| No. | R² | Weight | [M + 1]⁺ | Analogous to |
| 201 | 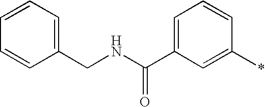 | 476.56 | 477 | Example No. 197 |
| 202 | 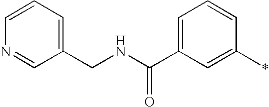 | 477.54 | 478 | Example No. 197 |
| 203 | 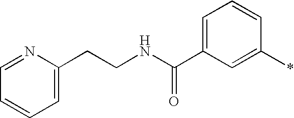 | 491.57 | 492 | Example No. 197 |
| 204 | 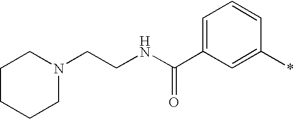 | 497.62 | 498 | Example No. 197 |
| 205 | 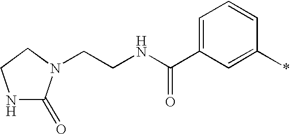 | 498.56 | 499 | Example No. 197 |
| 206 | 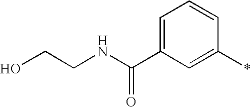 | 430.48 | 431 | Example No. 197 |
| 207 | 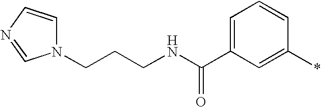 | 494.57 | 495 | Example No. 197 |
| 208 | 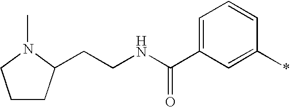 | 497.62 | 498 | Example No. 197 |
| 209 | 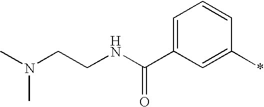 | 457.55 | 458 | Example No. 197 |
| 210 | 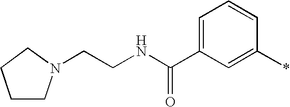 | 483.59 | 484 | Example No. 197 |
| 211 | 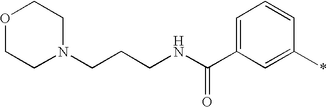 | 513.62 | 514 | Example No. 197 |
| 212 | 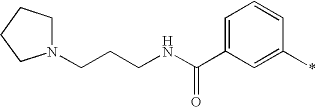 | 497.62 | 498 | Example No. 197 |
| 213 | 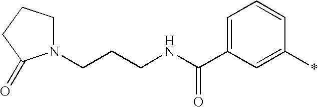 | 511.60 | 512 | Example No. 197 |
| 214 | 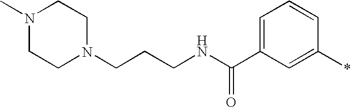 | 526.66 | 527 | Example No. 197 |
| 215 | 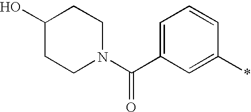 | 470.547 | 471 | Example No. 197 |
| 216 | 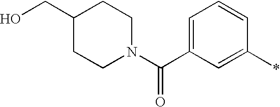 | 484.574 | 485 | Example No. 197 |
| 217 | 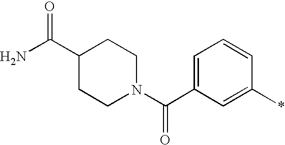 | 497.573 | 498 | Example No. 197 |
| 218 | 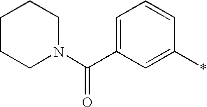 | 454.548 | 455 | Example No. 197 |
| 219 | 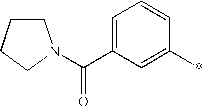 | 440.522 | 441 | Example No. 197 |

### EXAMPLE 220

### (E or Z)-Cyano-(3-ethyl-5-(E/Z)-{[4-(2-hydroxy-ethoxy)-phenylamino]-methylene}-4-oxo-thiazolidin-2-ylidene)-acetic acid ethyl ester

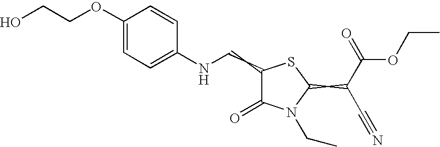

2 g of the compound that is described under Example c) and 1.14 g of the compound that is described under Example au) are introduced into 50 ml of ethanol and stirred under reflux for 4 hours. The reaction mixture is hot-filtered, and the solid is recrystallized from ethanol. 1.78 g of the title compound is obtained as a pH-dependent 5-(E/Z)-isomer mixture.

1H-NMR (DMSO-d6, stored with K₂CO₃, main isomer): δ=1.14-1.34 (6H); 3.70 (2H); 3.95 (2H); 4.15-4.32 (4H); 4.88 (1H); 6.94 (2H); 7.25 (2H); 8.12 (1H); 10.50 (1H) ppm.

### EXAMPLE 221

### (E or Z)-Cyano-(3-ethyl-5-(E/Z)-{[3-(2-methoxy-acetylamino)-phenylamino]-methylene}-4-oxo-thiazolidin-2-ylidene)-acetic acid ethyl ester

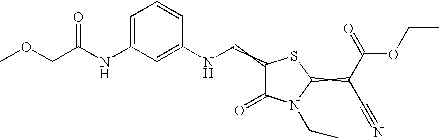

75 mg of the compound that is described under Example be) is dissolved in 5 ml of dichloromethane, mixed with 6 ml of 2-molar hydrochloric acid in diethyl ether and stirred for 18 hours at room temperature. The reaction mixture is evaporated to the dry state in a rotary evaporator and dissolved in 5 ml of ethanol. 93 μl of triethylamine and 63 mg of the compound that is described under Example c) are added and stirred under reflux for 7 hours. The reaction mixture is concentrated by evaporation, and after purification by chromatography on silica gel, 41 mg of the title compound is obtained as a pH-dependent 5-(E/Z)-isomer mixture.

1H-NMR (DMSO-d6, stored with K₂CO₃, main isomer): δ=1.14-1.33 (6H); 3.39 (3H); 4.00 (2H); 4.15-4.32 (4H); 6.96 (1H); 7.25 (1H); 7.33 (1H); 7.72 (1H); 8.15 (1H); 9.80 (1H); 10.65 (1H) ppm.

### EXAMPLE 222

### (E or Z)-Cyano-(3-ethyl-5-(E/Z)-{[3-(3-morpholin-4-yl-propionylamino)-phenylamino]-methylene}-4-oxo-thiazolidin-2-ylidene)-acetic acid ethyl ester

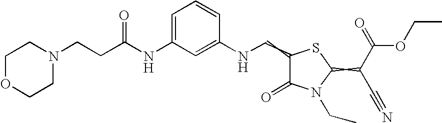

92 mg of the compound that is described under Example bg) is dissolved in 4 ml of dichloromethane, mixed with 5 ml of 2-molar hydrochloric acid in diethyl ether and stirred for 18 hours at room temperature. The reaction mixture is evaporated to the dry state in a rotary evaporator and dissolved in 3 ml of ethanol. 166 μl of triethylamine and 60 mg of the compound that is described under Example c) are added and stirred under reflux for 4 hours. The reaction mixture is concentrated by evaporation, mixed with water and extracted with dichloromethane. The solution is concentrated by evaporation, and after purification by chromatography on silica gel, 65 mg of the title compound is obtained as a pH-dependent 5-(E/Z)-isomer mixture.

1H-NMR (DMSO-d6, stored with K₂CO₃, main isomer): δ=1.19-1.35 (6H); 2.35-2.46 (6H); 3.40 (2H); 3.58 (4H); 4.18-4.33 (4H); 7.40-7.50 (2H); 7.51-7.59 (1H); 7.75 (1H); 8.53 (1H); 10.64 (1H) ppm.

### EXAMPLE 223

### (E or Z)-Cyano-(3-ethyl-5-(E/Z)-{[3-(2-morpholin-4-yl-ethanesulfonylamino)-phenylamino]-methylene}-4-oxo-thiazolidin-2-ylidene)-acetic acid ethyl ester

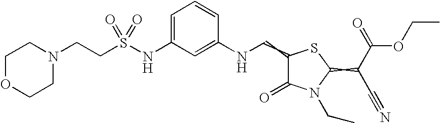

52 mg of the compound that is described under Example bi) is dissolved in 3 ml of dichloromethane, mixed with 6 ml of 2-molar hydrochloric acid in diethyl ether, and stirred for 18 hours at room temperature. The reaction mixture is evaporated to the dry state in a rotary evaporator and dissolved in 3 ml of ethanol. 55 μl of triethylamine and 30 mg of the compound that is described under Example c) are added and stirred under reflux for 7 hours. The reaction mixture is concentrated by evaporation, mixed with water and extracted with dichloromethane. The solution is concentrated by evaporation, and after purification by chromatography on silica gel, 11 mg of the title compound is obtained as a pH-dependent 5-(E/Z)-isomer mixture.

1H-NMR (DMSO-d6, stored with K₂CO₃, main isomer): δ=1.16-1.31 (6H); 2.29 (4H); 2.67 (2H); 3.20-3.34 (2H); 3.47 (4H); 4.16-4.30 (4H); 6.90 (1H); 7.01 (1H); 7.1 (1H); 7.28 (1H); 8.14 (1H); 9.93 (1H); 10.61 (1H); ppm.

Analogously to Examples 221, 222 and 223), the following compounds are produced from the intermediate product that is described under Example c):

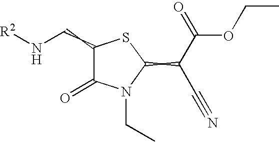

| Example | | Molecular | MS (ESI) | Synthesis |
|---|---|---|---|---|
| No. | R² | Weight | [M + 1]⁺ | Analogous to |
| 224 | 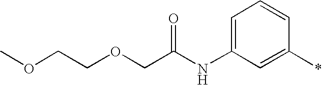 | 474.535 | 475 | Example No. 221 |
| 225 | 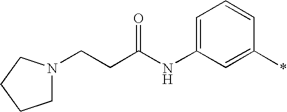 | 483.59 | 484 | Example No. 222 |
| 226 | 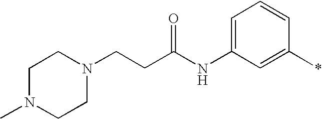 | 512.632 | 513 | Example No. 222 |
| 227 | 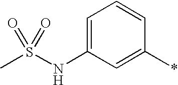 | 436.511 | 437 | Example No. 221 |
| 228 | 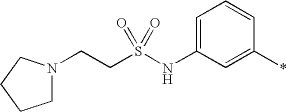 | 519.644 | 520 | Example No. 223 |
| 229 | 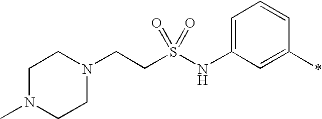 | 548.686 | 549 | Example No. 223 |
| 230 | 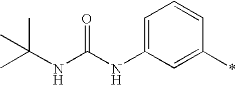 | 457.552 | 458 | Example No. 221 |
| 231 | 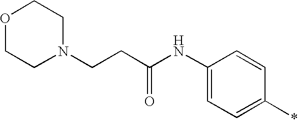 | 499.589 | 500 | Example No. 222 |
| 232 | 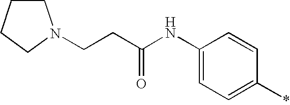 | 483.59 | 484 | Example No. 222 |
| 233 | 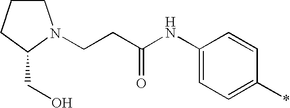 | 513.616 | 514 | Example No. 222 |
| 234 | 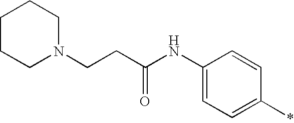 | 497.617 | 498 | Example No. 222 |
| 235 | 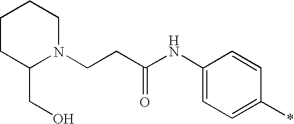 | 527.643 | 528 | Example No. 222 |
| 236 | 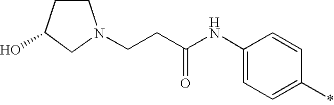 | 499.589 | 500 | Example No. 222 |
| 237 | 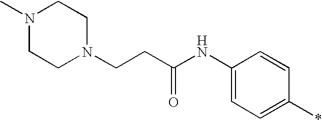 | 512.632 | 513 | Example No. 222 |
| 238 | 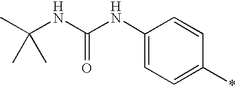 | 457.552 | 458 | Example No. 221 |

Analogously to Example 160), the following compounds are produced from the intermediate product that is described under Example c):

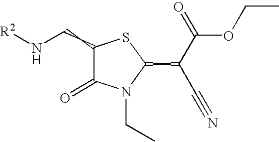

| Example | | Molecular | MS (ESI) | Synthesis |
|---|---|---|---|---|
| No. | R² | Weight | [M + 1]⁺ | Analogous to |
| 239 | 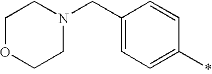 | 442.537 | 443 | Example No. 160 |
| 240 | 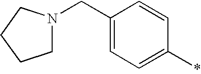 | 426.538 | 427 | Example No. 160 |
| 241 | 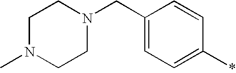 | 455.58 | 456 | Example No. 160 |
| 242 | 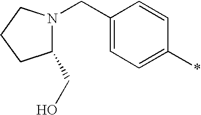 | 456.564 | 457 | Example No. 160 |

Analogously to Example 178), the following compounds are produced from the intermediate product that is described under Example ba):

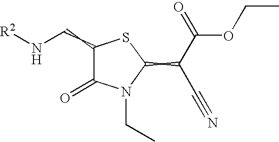

| Example | | Molecular | MS (ESI) | Synthesis |
|---|---|---|---|---|
| No. | R² | Weight | [M + 1]⁺ | Analogous to |
| 243 | 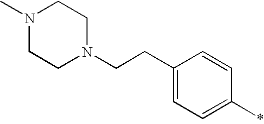 | 469.607 | 470 | Example No. 178 |
| 244 | 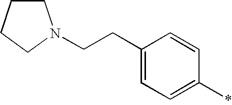 | 440.565 | 441 | Example No. 178 |
| 245 | 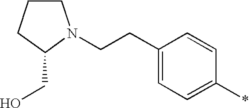 | 470.591 | 471 | Example No. 178 |
| 246 | 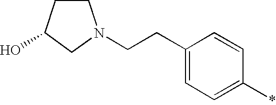 | 456.564 | 457 | Example No. 178 |
| 247 | 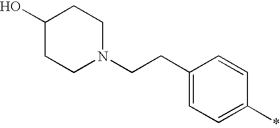 | 470.591 | 471 | Example No. 178 |
| 248 | 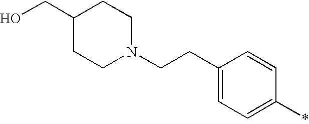 | 484.618 | 485 | Example No. 178 |
| 249 | 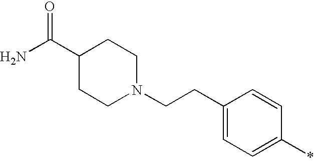 | 497.617 | 498 | Example No. 178 |
| 250 | 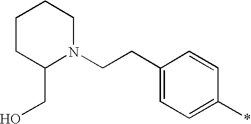 | 484.618 | 485 | Example No. 178 |
| 251 | 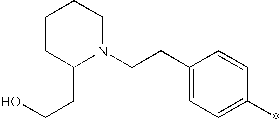 | 498.645 | 499 | Example No. 178 |
| 252 | 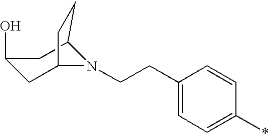 | 496.629 | 497 | Example No. 178 |
| 253 | 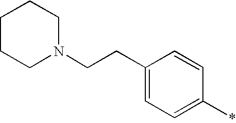 | 454.592 | 455 | Example No. 178 |
| 254 | 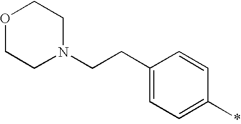 | 456.564 | 457 | Example No. 178 |

### EXAMPLE 255

### (E or Z)-Cyano-(3-ethyl-5-(E/Z)-{[4-(3-morpholin-4-yl-propoxy)-phenylamino]-methylene}-4-oxo-thiazolidin-2-ylidene)-acetic acid ethyl ester

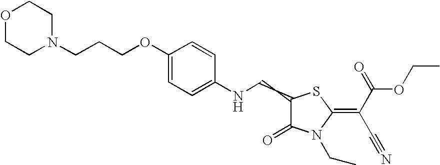

130 mg of the compound that is described under Example bc) is dissolved in 5 ml of dichloromethane, mixed with 3 ml of 2-molar hydrochloric acid in diethyl ether, and stirred for 18 hours at room temperature. The reaction mixture is evaporated to the dry state in a rotary evaporator and dissolved in 3 ml of ethanol. 168 μl of triethylamine and 89 mg of the compound that is described under Example c) are added and stirred for 4 hours under reflux. The reaction mixture is concentrated by evaporation, mixed with water and extracted with dichloromethane. The solution is concentrated by evaporation, and after purification by chromatography on silica gel, 33 mg of the title compound is obtained as a pH-dependent 5-(E/Z)-isomer mixture.

1H-NMR (DMSO-d6, stored with K₂CO₃, main isomer): δ=1.15-1.30 (6H); 1.85 (2H); 2.29-2.45 (6H); 3.58 (4H); 3.97 (2H); 4.16-4.30 (4H); 6.95 (2H); 7.25 (2H); 8.12 (1H); 10.48 (1H); ppm.

### EXAMPLE 256

### (E or Z)-Cyano-{3-cyclopropyl-4-oxo-5-(E/Z)-[(3,4,5-trimethoxy-phenylamino)-methylene]-thiazolidin-2-ylidene}-acetic acid ethyl ester

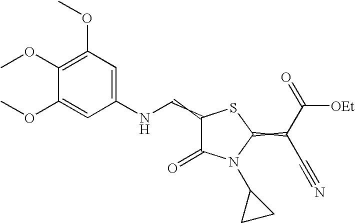 Process Variant C

A solution of 31 mg of the substance that is described under Example ay) and 18 mg of 3,4,5-trimethoxyaniline in 1 ml DMSO is shaken for 6 hours at 100° C. Ethyl acetate and a semi-saturated aqueous ammonium chloride solution are added. The mixture is extracted with ethyl acetate. The crude product that is obtained after the organic solvent is evaporated is purified by HPLC. 4 mg of the title compound is obtained as a pH-dependent 5-(E/Z)-isomer mixture.

¹H-NMR (DMSO-d6): δ=1.00 (2H), 1.18 (2H), 1.28 (3H), 3.02 (1H), 3.61 (3H), 3.81 (6H), 4.23 (2H), 6.63 (2H), 6.78 (2H, Z-isomer), 8.18 (1H), 8.42 (1H, Z-isomer), 11.10 (1H), 11.20 (1H, Z-isomer) ppm.

### EXAMPLE 257

### (E or Z)-Cyano-{3-ethyl-5-(E/Z)-[(1H-indazol-6-ylamino)-methylene]-4-oxo-thiazolidin-2-ylidene}-acetic acid ethyl ester

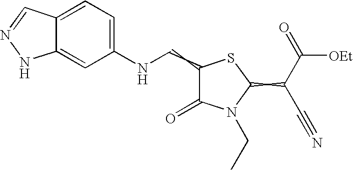 Process Variant D

A solution of 30 mg of the substance that is described under Example c) and 13 mg of 6-aminoindazole in 1 ml of DMSO is shaken for 6 hours at 100° C. The reaction mixture that is obtained is purified directly by HPLC. 8 mg of the title compound is obtained as a pH-dependent 5-(E/Z)-isomer mixture.

¹H-NMR (DMSO-d6): δ=1.28 (6H), 4.27 (4H), 6.75 (2H), 7.13 (1H), 7.40 (1H), 7.55 (1H, Z-isomer), 7.72 (1H), 8.00 (1H), 8.28 (1H), 8.59 (1H, Z-isomer), 11.31 (1H), 12.46 (1H), 12.55 (1H, Z-isomer) ppm.

### EXAMPLE 258

### (E or Z)-Cyano-{3-Butyl-5-(E/Z)-[(6-methoxy-pyridin-3-ylamino)-methylene]-4-oxo-thiazolidin-2-ylidene}-acetic acid ethyl ester

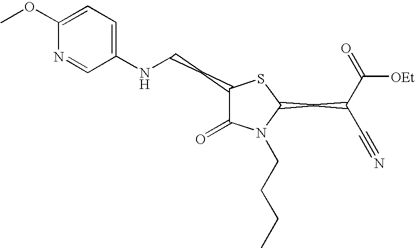

Analogously to Example 63, process variant C, 12 mg of the title compound is obtained as a pH-dependent 5-(E/Z)-isomer mixture from 31 mg of the N-n-butyl derivative that is produced analogously to Example c) and 12 mg of 2-methoxy-5-amino-pyridine in 1 ml of DMSO.

¹H-NMR (DMSO-d6): δ=0.91 (3H), 1.27 (3H), 1.32 (2H), 1.61 (2H), 3.82 (3H), 4.2 (4H), 6.82 (1H), 7.77 (1H), 8.15 (2H), 11.25 (1H), 11.30 ppm.

### EXAMPLE 259

### (E or Z)-Cyano-(3-cyclopropyl-5-(E/Z)-{[4-(4-methylamino-benzyl)-phenylamino]-methylene}-4-oxo-thiazolidin-2-ylidene)-acetic acid ethyl ester

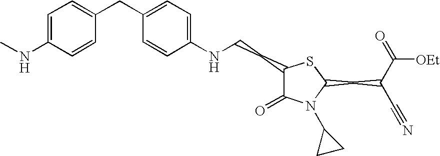

Analogously to Example 63, process variant C, 10 mg of the title compound is obtained as a pH-dependent 5-(E/Z)-isomer mixture from 31 mg of the substance that is described under Example yc) and 22 mg of 4-(4-N-methylaminobenzyl-)-phenylamine in 1 ml of DMSO.

¹H-NMR (DMSO-d6): δ=1.0 (2H), 1.15 (2H), 1.28 (3H), 2.62 (3H), 3.02 (1H), 3.74 (2H), 4.23 (2H), 5.43 (1H), 6.46 (2H), 6.93 (2H), 7.16 (4H), 8.05 (1H), 8.35 (1H, Z-isomer), 11.16 (1H), 11.25 (1H, Z-isomer) ppm.

### EXAMPLE 260

### (E or Z)-Cyano-[3-cyclopropyl-4-oxo-5-(E/Z)-(thiazol-2-ylamino-methylene)-thiazolidin-2-ylidene]-acetic acid ethyl ester

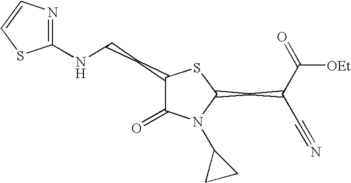

Analogously to Example 63, process variant C, 7 mg of the title compound is obtained as a pH-dependent 5-(E/Z)-isomer mixture from 31 mg of the substance that is described under Example yc) and 10 mg of 2-aminothiazole in 1 ml of DMSO.

¹H-NMR (DMSO-d6): δ=1.02 (2H), 1.18 (2H), 1.28 (3H), 3.04 (1H), 4.22 (2H), 7.20 (1H), 7.39 (1H), 8.22 (1H, 11.86 (1H) ppm.

### EXAMPLE 261

### (E or Z)-Cyano-(3-cyclopropyl-4-oxo-5 (E/Z)-phenylamino-methylene-thiazolidin-2-ylidene)-acetic acid ethyl ester

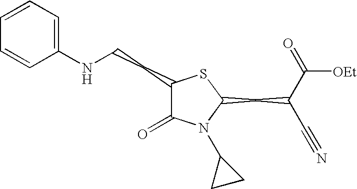

Analogously to Example 1, process variant B, 94 mg of the title compound is obtained as a pH-dependent 5-(E/Z)-isomer mixture from 154 mg of the substance that is described under Example yc) and 52 mg of aniline in 5 ml of EtOH.

¹H-NMR (DMSO-d6): δ=1.10 (2H), 1.17 (2H), 1.28 (3H), 3.03 (1H), 4.22 (2H), 7.08 (1H), 7.31 (4H), 8.11 (1H), 8.41 (1H, Z-isomer), 10.39 (1H), 10.51 (1H, Z-isomer) ppm.

### EXAMPLE 262

### (E or Z)-Cyano-[3-cyclopropyl-5-(E/Z)-({4-[2-(2-hydroxy-ethoxy)-ethoxy]-phenylamino}-methylene)-4-oxo-thiazolidin-2-ylidene]-acetic acid ethyl ester

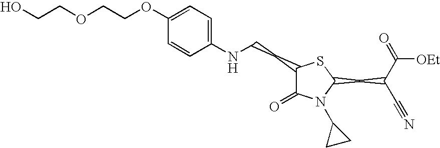

Analogously to Example 1, process variant B, 160 mg of the title compound is obtained as a pH-dependent 5-(E/Z)-isomer mixture from 154 mg of the substance that is described under Example yc) and 111 mg of 2-[2-(4-amino-phenoxy)-ethoxy]-ethanol in 5 ml of EtOH.

¹H-NMR (DMSO-d6): δ=0.99 (2H), 1.17 (2H), 1.25 (3H), 3.02 (1H), 3.49 (4H), 3.72 (2H), 4.07 (2H), 4.22 (2H), 4.62 (1H), 6.93 (2H), 7.23 (2H), 7.32 (2H, Z-isomer), 8.02 (1H), 8.31 (1H, Z-isomer), 10.31 (1H), 10.51 (1H, Z-isomer) ppm.

### EXAMPLE 263

### 6-{[2-(E or Z)-(Cyano-ethoxycarbonyl-methylene)-3-cyclopropyl-4-oxo-thiazolidin-5-(E,Z)-ylidene-methyl]-amino}-naphthalene-2-carboxylic acid

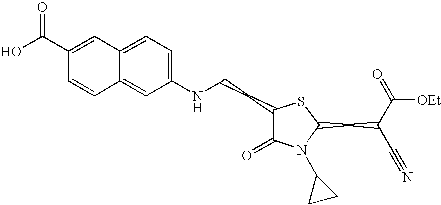

Analogously to Example 1, process variant B, 147 mg of the title compound is obtained as a pH-dependent 5-(E/Z)-isomer mixture from 154 mg of the substance that is described under Example yc) and 105 mg of 6-amino-naphthalene-2-carboxylic acid in 5 ml of EtOH.

¹H-NMR (DMSO-d6): δ=1.02 (2H), 1.20 (2H), 1.28 (3H), 3.08 (1H), 4.24 (2H), 7.59 (1H), 7.36 (1H), 7.92 (2H), 8.08 (1H), 8.29 (1H), 8.52 (1H), 10.62 (1H), 10.70 (1H, Z-isomer), 12.96 (1H) ppm.

### EXAMPLE 264

### (E or Z)-Cyano-{3-isobutyl-4-oxo-5-(E/Z)-[(3,4,5-trimethoxy-phenylamino)-methylene]-thiazolidin-2-ylidene}-acetic acid ethyl ester

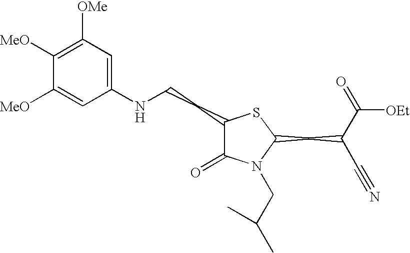

Analogously to Example 63, process variant C, 9 mg of the title compound is obtained as a pH-dependent 5-(E/Z)-isomer mixture from 32 mg of the N-iso-butyl derivative that is produced analogously to Example c) and 18 mg of 3,4,5-trimethoxyaniline in 1 ml of DMSO.

¹H-NMR (DMSO-d6): δ=0.88 (6H), 1.27 (3H), 2.12 (1H), 3.63 (3H), 3.81 (6H), 4.06 (2H), 4.22 (2H), 6.67 (2H), 6.78 (2H, Z-isomer), 8.30 (1H), 8.54 (1H, Z-isomer), 11.20 (1H), 11.25 ppm.

### EXAMPLE 265

### (E or Z)-Cyano-[3-isobutyl-4-oxo-5-(E/Z)-(thiazol-2-ylamino-methylene)-thiazolidin-2-ylidene]-acetic acid ethyl ester

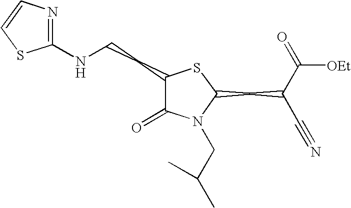

Analogously to Example 63, process variant C, 5 mg of the title compound is obtained as a pH-dependent 5-(E/Z)-isomer mixture from 32 mg of the N-iso-butyl derivative that is produced analogously to Example c) and 10 mg of 2-aminothiazole in 1 ml of DMSO.

¹H-NMR (DMSO-d6): δ=0.89 (6H), 1.28 (3H), 2.12 (1H), 4.05 (2H), 4.24 (2H), 7.25 (1H), 7.42 (1H), 8.32 (1H, 11.95 (1H) ppm.

### EXAMPLE 266

### (E or Z)-Cyano-{3-isobutyl-(E/Z)-5-[(6-methoxy-pyridin-3-ylamino)-methylene]-4-oxo-thiazolidin-2-(Z)-ylidene}-acetic acid ethyl ester

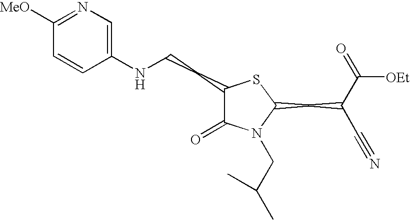

Analogously to Example 63, process variant C, 8 mg of the title compound is obtained as a pH-dependent 5-(E/Z)-isomer mixture from 32 mg of the N-iso-butyl derivative that is produced analogously to Example c) and 13 mg of 2-methoxy-4-amino-pyridine in 1 ml of DMSO.

¹H-NMR (DMSO-d6): δ=0.88 (6H), 1.27 (3H), 2.12 (1H), 3.82 (3H), 4.08 (2H), 4.22 (2H), 6.82 (2H), 7.78 (1H), 8.18 (2H), 8.31 (2H, Z-isomer), 11.25 (1H), 11.30 (1H, Z-isomer) ppm.

### EXAMPLE 267

### (E or Z)-Cyano-(3-ethyl-5-(E/Z)-{[4-(4-methylamino-benzyl)-phenylamino]-methylene}-4-oxo-thiazolidin-2-ylidene)-acetic acid ethyl ester

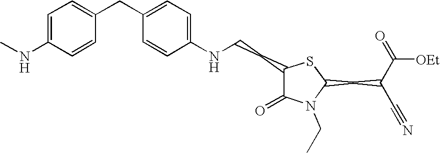

Analogously to Example 64, process variant D, 9 mg of the title compound is obtained as a pH-dependent 5-(E/Z)-isomer mixture from 30 mg of the substance that is described under Example c) and 21 mg of 4-(4-N-methylaminobenzyl-)-phenylamine in 1 ml of DMSO.

¹H-NMR (DMSO-d6): δ=1.22 (6H), 2.64 (3H), 3.73 (2H), 4.21 (4H), 6.51 (2H), 6.95 (2H), 7.19 (4H), 8.16 (1H), 8.42 (1H, Z-isomer), 11.25 (1H), 11.30 (1H, Z-isomer) ppm.

### EXAMPLE 268

### (E or Z)-Cyano-{3-ethyl-5-(E/Z)-[(4-hydroxy-phenylamino)-methylene]-4-oxo-thiazolidin-2-ylidene}-acetic acid ethyl ester

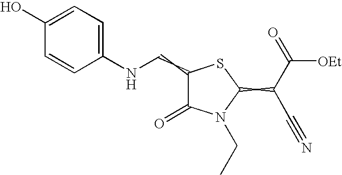

Analogously to Example 1, process variant B, 37 mg of the title compound is obtained as a pH-dependent 5-(E/Z)-isomer mixture from 50 mg of the substance that is described under Example c) and 20 mg of 4-hydroxyaniline in 1 ml of EtOH.

¹H-NMR (DMSO-d6): δ=1.24 (6H), 4.20 (4H), 6.75 (2H), 7.15 (2H), 7.21 (2H, Z-isomer), 8.05 (1H), 8.35 (1H, Z-isomer), 9.40 (1H), 9.45 (1H, Z-isomer), 11.45 (1H), 11.60 (1H, Z-isomer) ppm.

Similarly produced according to process variant B), C) or D) are also the following compounds:

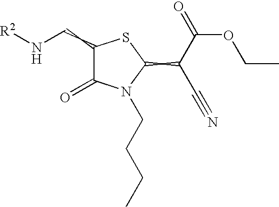

| | | Molecular | MS (ESI) |
|---|---|---|---|
| Ex. No. | R² | Weight | M + 1 |
| 269 | 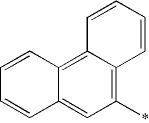 | 471.58 | 472 |
| 270 | 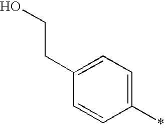 | 415.51 | 416 |
| 271 | 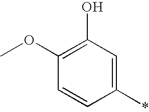 | 417.48 | 418 |
| 272 | 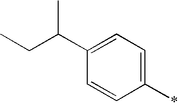 | 427.57 | 428 |
| 273 | 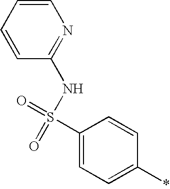 | 527.62 | 528 |
| 274 | 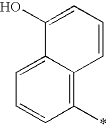 | 437.52 | 438 |
| 275 | 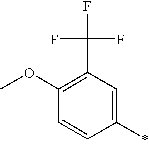 | 469.48 | 470 |
| 276 | 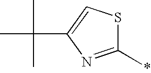 | 434.58 | 435 |
| 277 | 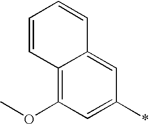 | 451.54 | 452 |
| 278 | 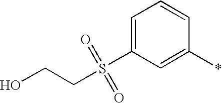 | 479.58 | 480 |
| 279 | 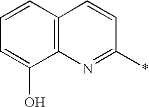 | 438.51 | 439 |
| 280 | 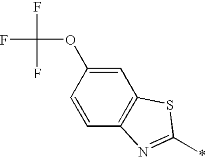 | 512.53 | 513 |
| 281 | 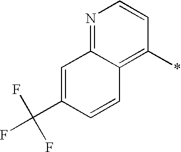 | 490.50 | 491 |
| 282 | 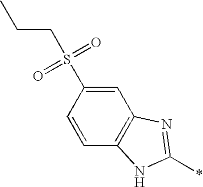 | 517.63 | 518 |
| 283 | 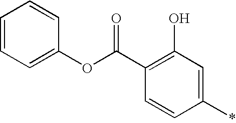 | 507.57 | 508 |
| 284 | 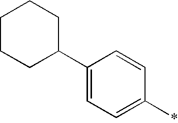 | 453.60 | 454 |
| 285 | 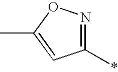 | 376.44 | 377 |
| 286 | 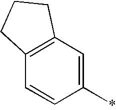 | 411.52 | 412 |
| 287 | 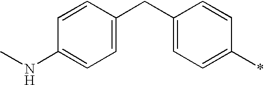 | 490.63 | 491 |
| 288 | 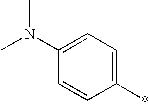 | 414.53 | 415 |
| 289 | 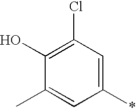 | 435.93 | 436 |
| 290 | 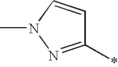 | 375.45 | 376 |
| 291 | 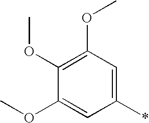 | 461.54 | 462 |
| 292 | 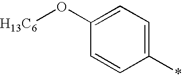 | 471.62 | 472 |
| 293 | 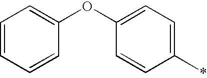 | 463.56 | 464 |
| 294 | 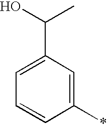 | 415.51 | 416 |
| 295 | 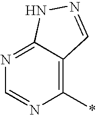 | 413.46 | 414 |
| 296 | 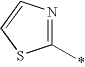 | 378.48 | 379 |
| 297 | 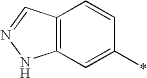 | 411.48 | 412 |
| 298 | 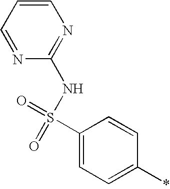 | 528.61 | 529 |
| 299 | 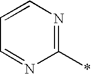 | 373.44 | 374 |
| 300 | 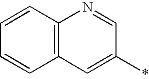 | 422.51 | 423 |
| 301 | 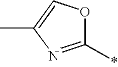 | 376.44 | 377 |
| 302 | 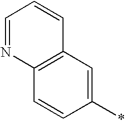 | 422.51 | 423 |
| 303 | 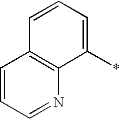 | 422.51 | 423 |
| 304 | 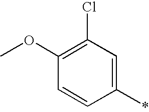 | 435.93 | 436 |
| 305 | 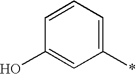 | 387.46 | 388 |
| 306 | 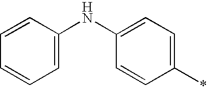 | 462.57 | 463 |
| 307 | 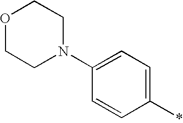 | 456.56 | 457 |
| 308 | 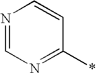 | 373.44 | 374 |
| 309 | 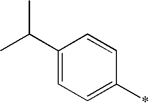 | 413.54 | 414 |
| 310 | 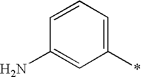 | 386.47 | 387 |
| 311 | 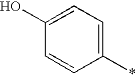 | 387.46 | 388 |

Similarly produced according to process variant B), C) or D) are also the following compounds:

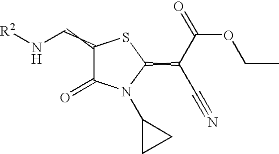

| Ex. | | Molecular | MS (ESI) |
|---|---|---|---|
| No. | R² | Weight | M + 1 |
| 312 | 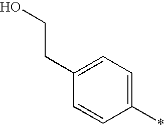 | 399.47 | 400 |
| 313 | 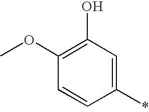 | 401.44 | 402 |
| 314 | 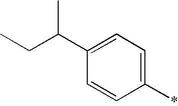 | 411.52 | 412 |
| 315 | 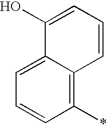 | 421.48 | 422 |
| 316 | 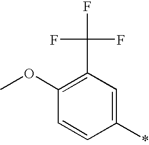 | 453.44 | 454 |
| 317 | 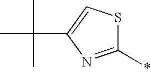 | 418.54 | 419 |
| 318 | 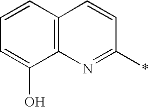 | 422.46 | 423 |
| 319 | 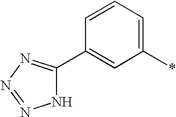 | 423.46 | 424 |
| 320 | 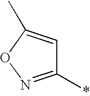 | 360.39 | 361 |
| 321 | 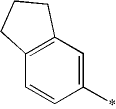 | 395.48 | 396 |
| 322 | 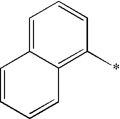 | 405.48 | 406 |
| 323 | 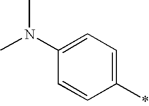 | 398.49 | 399 |
| 324 | 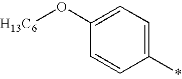 | 455.58 | 456 |
| 325 | 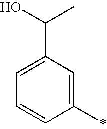 | 399.47 | 400 |
| 326 | 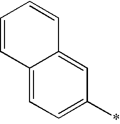 | 405.48 | 406 |
| 327 | 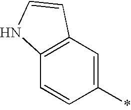 | 394.45 | 395 |
| 328 | 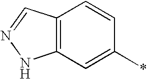 | 395.44 | 396 |
| 329 | 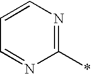 | 357.39 | 358 |
| 330 | 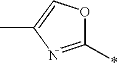 | 360.39 | 361 |
| 331 | 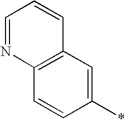 | 406.46 | 407 |
| 332 | 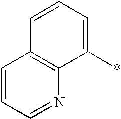 | 406.46 | 407 |
| 333 | 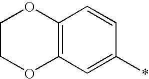 | 413.45 | 414 |
| 334 | 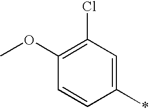 | 419.89 | 420 |
| 335 | 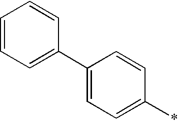 | 431.51 | 432 |
| 336 | 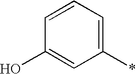 | 371.42 | 372 |
| 337 | 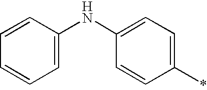 | 446.53 | 447 |
| 338 | 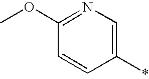 | 386.43 | 387 |
| 339 | 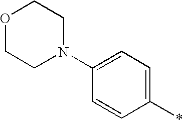 | 440.52 | 441 |
| 340 | 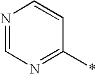 | 357.39 | 358 |
| 341 | 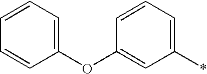 | 447.51 | 448 |
| 342 | 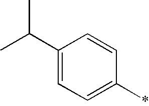 | 397.50 | 398 |
| 343 | 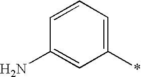 | 370.43 | 371 |
| 344 | 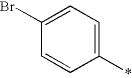 | 434.31 | 435 |
| 345 | 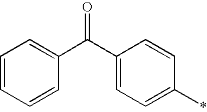 | 459.52 | 460 |
| 346 | 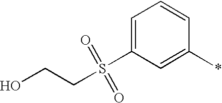 | 463.53 | 464 |
| 347 | 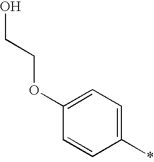 | 415.47 | 416 |
| 348 | 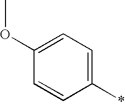 | 385.44 | 386 |
| 349 | 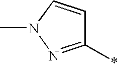 | 359.41 | 360 |
| 350 | 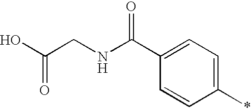 | 456.48 | 457 |
| 351 | 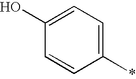 | 371.42 | 372 |

Similarly produced according to process variant B), C) or D) are also the following compounds:

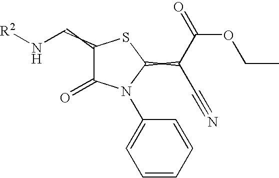

| Ex. | | Molecular | MS (ESI) |
|---|---|---|---|
| No. | R² | Weight | M + 1 |
| 352 | 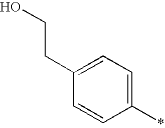 | 435.50 | 436 |
| 353 | 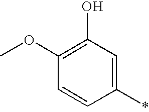 | 437.47 | 438 |
| 354 | 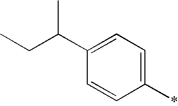 | 447.56 | 448 |
| 355 | 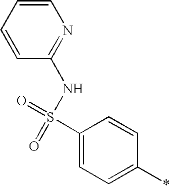 | 547.61 | 548 |
| 356 | 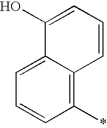 | 457.51 | 458 |
| 357 | 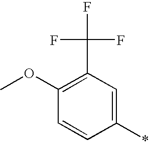 | 489.47 | 490 |
| 358 | 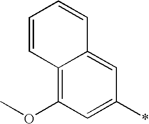 | 471.54 | 472 |
| 359 | 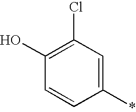 | 441.89 | 442 |
| 360 | 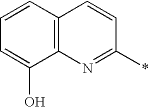 | 458.50 | 459 |
| 361 | 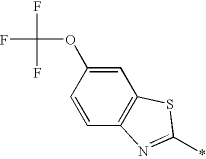 | 532.52 | 533 |
| 362 | 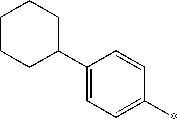 | 473.59 | 474 |
| 363 | 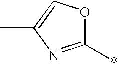 | 396.43 | 397 |
| 364 | 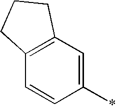 | 431.51 | 432 |
| 365 | 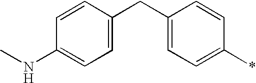 | 510.62 | 511 |
| 366 | 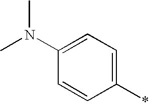 | 434.52 | 435 |
| 367 | 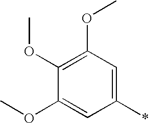 | 481.53 | 482 |
| 368 | 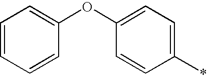 | 483.55 | 484 |
| 369 | 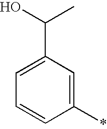 | 435.50 | 436 |
| 370 | 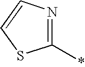 | 398.47 | 399 |
| 371 | 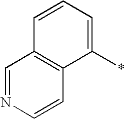 | 442.50 | 443 |
| 372 | 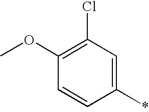 | 455.92 | 456 |
| 373 | 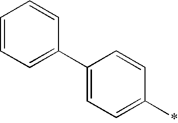 | 467.55 | 468 |
| 374 | 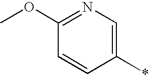 | 422.46 | 423 |
| 375 | 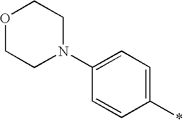 | 476.56 | 477 |
| 376 | 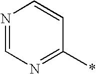 | 393.43 | 394 |
| 377 | 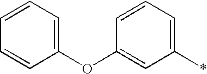 | 483.55 | 484 |
| 378 | 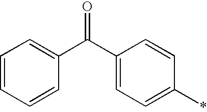 | 495.56 | 496 |
| 379 | 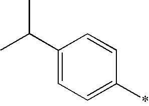 | 433.53 | 434 |
| 380 | 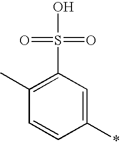 | 485.54 | 486 |

Similarly produced according to process variant B), C) or D) are also the following compounds:

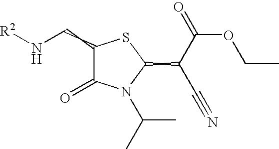

| Ex. | | Molecular | MS (ESI) |
|---|---|---|---|
| No. | R² | Weight | M + 1 |
| 381 | 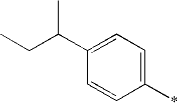 | 413.54 | 414 |
| 382 | 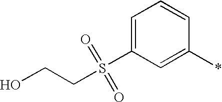 | 465.55 | 466 |
| 383 | 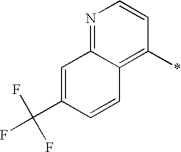 | 476.48 | 477 |
| 384 | 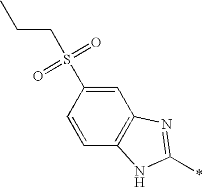 | 503.60 | 504 |
| 385 | 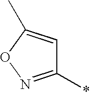 | 362.41 | 363 |
| 386 | 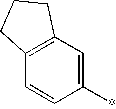 | 397.50 | 398 |
| 387 | 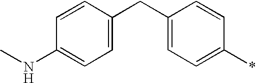 | 476.60 | 477 |
| 388 | 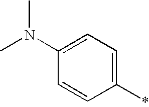 | 400.50 | 401 |
| 389 | 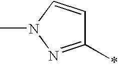 | 361.42 | 362 |
| 390 | 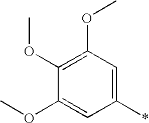 | 447.51 | 448 |
| 391 | 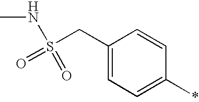 | 464.57 | 465 |
| 392 | 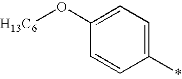 | 457.59 | 458 |
| 393 | 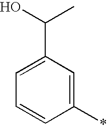 | 401.49 | 402 |
| 394 | 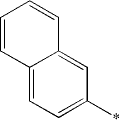 | 407.49 | 408 |
| 395 | 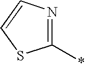 | 364.45 | 365 |
| 396 | 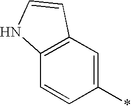 | 396.47 | 397 |
| 397 | 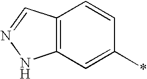 | 397.46 | 398 |
| 398 | 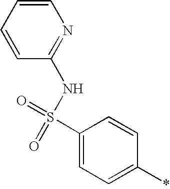 | 514.59 | 515 |
| 399 | 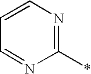 | 359.41 | 360 |
| 400 | 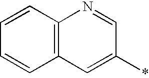 | 408.48 | 409 |
| 401 | 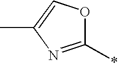 | 362.41 | 363 |
| 402 | 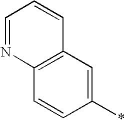 | 408.48 | 409 |
| 403 | 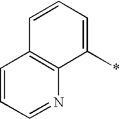 | 408.48 | 409 |
| 404 | 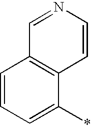 | 408.48 | 409 |
| 405 | 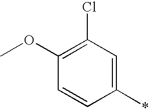 | 421.90 | 422 |
| 406 | 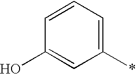 | 373.43 | 374 |
| 407 | 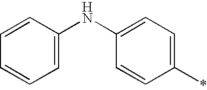 | 448.55 | 449 |
| 408 | 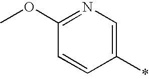 | 388.45 | 389 |
| 409 | 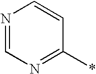 | 359.41 | 360 |
| 410 | 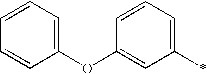 | 449.53 | 450 |
| 411 | 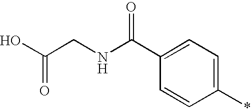 | 458.49 | 459 |
| 412 | 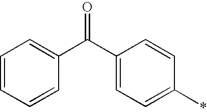 | 461.54 | 462 |
| 413 | 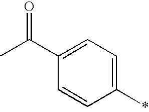 | 399.47 | 400 |

Similarly produced according to process variant B), C) or D) are also the following compounds:

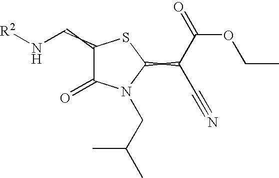

| Ex. | | Molecular | MS (ESI) |
|---|---|---|---|
| No. | R² | Weight | M + 1 |
| 414 | 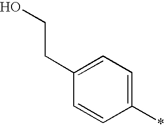 | 415.51 | 416 |
| 415 | 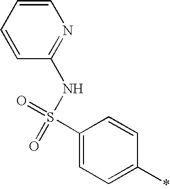 | 527.62 | 528 |
| 416 | 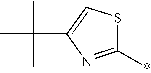 | 434.58 | 435 |
| 417 | 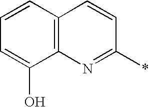 | 438.51 | 439 |
| 418 | 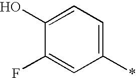 | 405.45 | 406 |
| 419 | 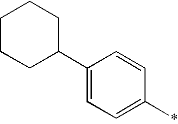 | 453.60 | 454 |
| 420 | 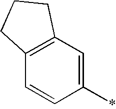 | 411.52 | 412 |
| 421 | 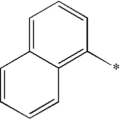 | 421.52 | 422 |
| 422 | 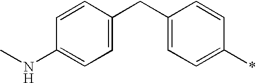 | 490.63 | 491 |
| 423 | 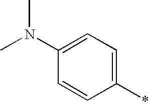 | 414.53 | 415 |
| 424 | 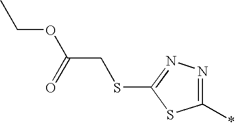 | 497.62 | 498 |
| 425 | 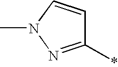 | 375.45 | 376 |
| 426 | 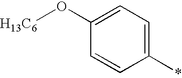 | 471.62 | 472 |
| 427 | 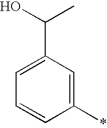 | 415.51 | 416 |
| 428 | 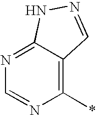 | 413.46 | 414 |
| 429 | 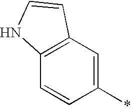 | 410.50 | 411 |
| 430 | 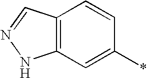 | 411.48 | 412 |
| 431 | 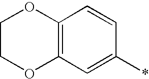 | 415.47 | 416 |
| 432 | 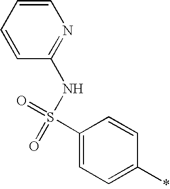 | 528.61 | 529 |
| 433 | 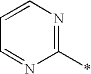 | 373.44 | 374 |
| 434 | 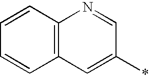 | 422.51 | 423 |
| 435 | 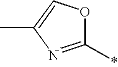 | 376.44 | 377 |
| 436 | 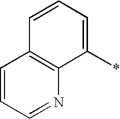 | 422.51 | 423 |
| 437 | 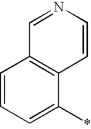 | 422.51 | 423 |
| 438 | 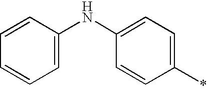 | 462.57 | 463 |
| 439 | 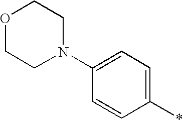 | 456.56 | 457 |
| 440 | 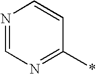 | 373.44 | 374 |
| 441 | 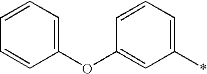 | 463.56 | 464 |

Similarly produced according to process variant C) or D) are also the following compounds:

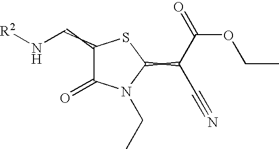

| Ex. | | Molecular | MS (ESI) |
|---|---|---|---|
| No. | R² | Weight | M + 1 |
| 442 | 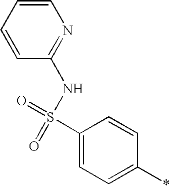 | 499.57 | 500 |
| 443 | 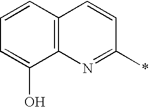 | 410.45 | 411 |
| 444 | 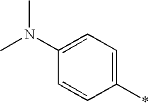 | 386.47 | 387 |
| 445 | 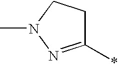 | 347.40 | 348 |
| 446 | 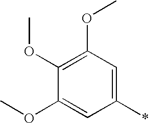 | 433.48 | 434 |
| 447 | 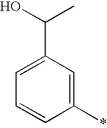 | 387.46 | 388 |
| 448 | 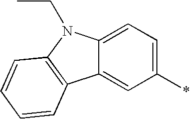 | 460.56 | 461 |
| 449 | 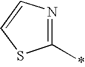 | 350.42 | 351 |
| 450 | 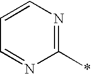 | 345.38 | 346 |
| 451 | 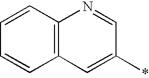 | 394.45 | 395 |
| 452 | 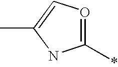 | 348.38 | 349 |
| 453 | 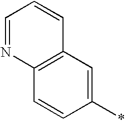 | 394.45 | 395 |
| 454 | 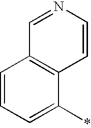 | 394.45 | 395 |
| 455 | 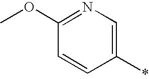 | 374.42 | 375 |
| 456 | 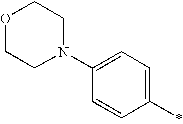 | 428.51 | 429 |
| 457 | 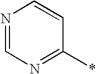 | 345.38 | 346 |
| 458 | 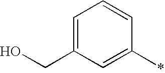 | 373.43 | 374 |
| 459 | 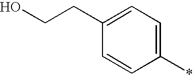 | 387.46 | 388 |
| 460 | 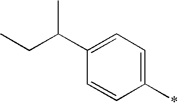 | 399.51 | 400 |
| 461 | 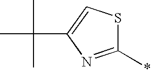 | 406.53 | 407 |
| 462 | 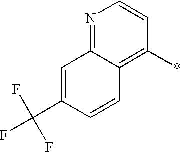 | 462.4 5 | 463 |
| 463 | 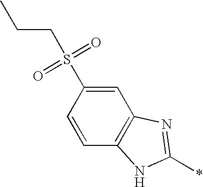 | 489.58 | 490 |
| 464 | 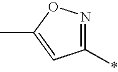 | 348.38 | 349 |
| 465 | 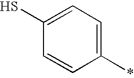 | 375.47 | 376 |
| 466 | 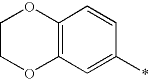 | 401.44 | 402 |
| 467 | 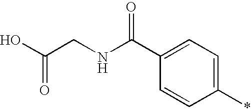 | 444.47 | 445 |
| 468 | 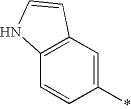 | 382.44 | 383 |
| 469 | 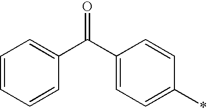 | 447.51 | 448 |
| 470 | 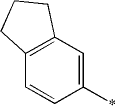 | 383.47 | 384 |
| 471 | 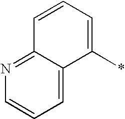 | 394.45 | 395 |
| 472 | 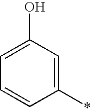 | 359.40 | 360 |
| 473 | 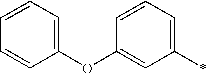 | 435.50 | 436 |
| 474 | 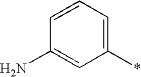 | 358.42 | 359 |
| 475 | 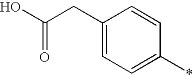 | 401.44 | 402 |
| 476 | 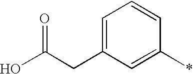 | 401.44 | 402 |
| 477 | 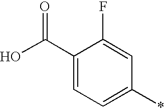 | 405.41 | 406 |
| 478 | 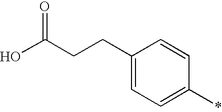 | 415.47 | 416 |

### EXAMPLE 479

### (E or Z)-Cyano-(3-ethyl-5-(E/Z)-{[4-(3-morpholin-4-yl-propylcarbamoyl)-phenylamino]-methylene}-4-oxo-thiazolidin-2-ylidene)-acetic acid ethyl ester

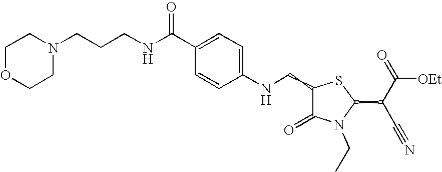

First, a solution of 0.018 ml of triethylamine and 42 mg of TBTU in 0.5 ml of DMF is added to a suspension of 39 mg of the compound, described in Example 25), in 1 ml of DMF. Then, 19 mg of N-(3-aminopropyl)-morpholine in 0.5 ml of DMF is added. The mixture is shaken overnight at room temperature. The solvent is evaporated, and the crude product that is obtained by preparative HPLC is purified. 11 mg of the title compound is obtained as a pH-dependent 5-(E/Z)-isomer mixture.

¹H-NMR (DMSO-d6): δ=1.32-1.48 (6H); 1.77-1.90 (2H); 2.52-2.68 (6H); 3.58 (2H); 3.70-3.80 (4H); 4.23-4.35 (2H); 4.40-4.50 (2H); 7.1 (2H); 7.85 (2H); 8.03 (1H); 9.00 (1H); 11.65 (1H) ppm.

### EXAMPLE 480

### (E or Z)-Cyano-{5-(E/Z)-[(4-{[(2-dimethylamino-ethyl)-methyl-carbamoyl]-methyl}-phenylamino)-methylene]-3-ethyl-4-oxo-thiazolidin-2-ylidene}-acetic acid ethyl ester

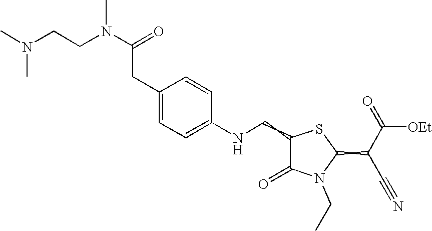

Produced in a way similar to Example 479, 25 mg of the title compound is obtained as a pH-dependent 5-(E/Z)-isomer mixture.

1H-NMR (DMSO-d6): δ=1.20-1.32 (6H); 2.80-2.88 (6H); 3.05 (3H); 3.20-3.26 (2H); 3.58-3.73 (4H); 4.18-4, 4.30 (4H); 7.21 (2H); 7.28 (2H); 8.18 (1H); 8.87 (1H); 10.53 (1H) ppm.

### EXAMPLE 481

### (E or Z)-Cyano-[5-({4-[2-(2-dimethylamino-1,1-dimethyl-ethylcarbamoyl)-ethyl]-phenylamino}-methylene)-3-ethyl-4-oxo-thiazolidin-2-ylidene]-acetic acid ethyl ester

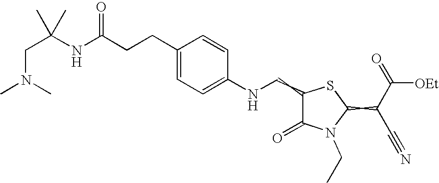

Produced in a way similar to Example 479, 17 mg of the title compound is obtained as a pH-dependent 5-(E/Z)-isomer mixture.

¹H-NMR (DMSO-d6): δ=0.90 (6H); 1.20-1.32 (6H); 2.65-2.90 (10H); 3.03 (2H); 1 (4H); 7.17-7.29 (4H); 7.28 (2H); 8.18 (1H); 8.80 (1H); 10.50 (1H) ppm.

Similarly produced are also the following compounds:

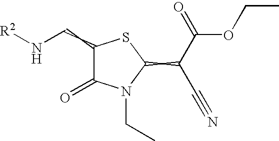

| Ex. | | Molecular | MS (ESI) |
|---|---|---|---|
| No. | R² | Weight | M + 1 |
| 482 | 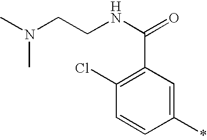 | 492.00 | 493 |
| 483 | 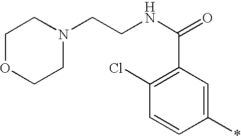 | 534.03 | 535 |
| 484 | 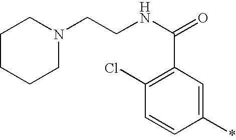 | 532.06 | 533 |
| 485 | 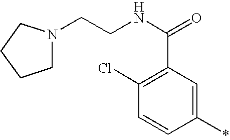 | 518.04 | 519 |
| 486 | 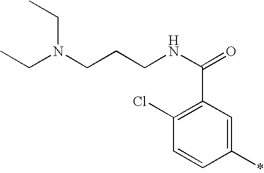 | 534.08 | 535 |
| 487 | 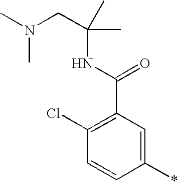 | 534.08 | 535 |
| 488 | 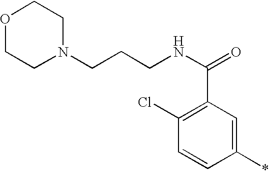 | 548.06 | 549 |
| 489 | 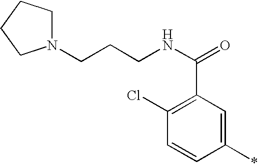 | 532.06 | 533 |
| 490 | 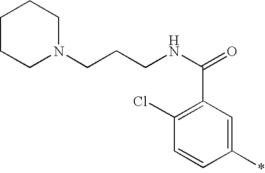 | 546.09 | 547 |
| 491 | 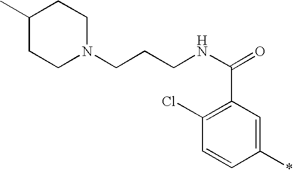 | 560.12 | 561 |
| 492 | 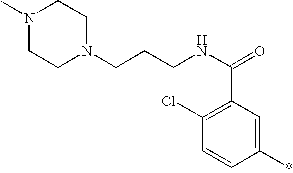 | 561.10 | 562 |
| 493 | 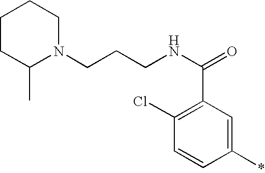 | 560.12 | 561 |
| 494 | 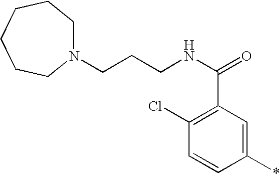 | 560.12 | 561 |
| 495 | 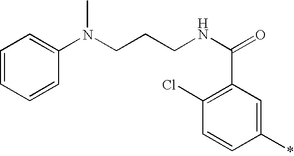 | 568.10 | 569 |
| 496 | 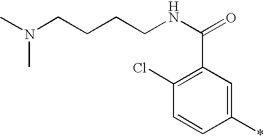 | 520.05 | 521 |
| 497 | 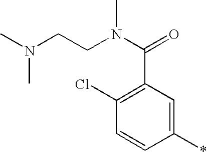 | 506.02 | 507 |
| 498 | 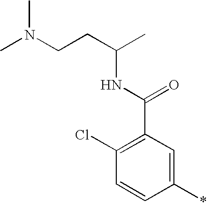 | 562.13 | 563 |
| 499 | 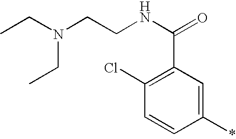 | 520.05 | 521 |
| 500 | 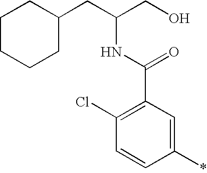 | 561.10 | 562 |
| 501 | 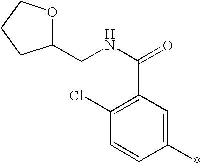 | 504.99 | 506 |
| 502 | 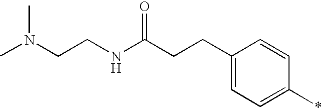 | 485.61 | 486 |
| 503 | 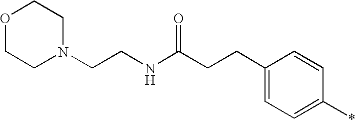 | 527.64 | 528 |
| 504 | 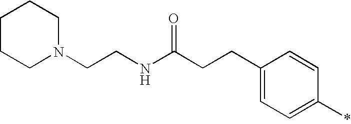 | 525.67 | 528 |
| 505 | 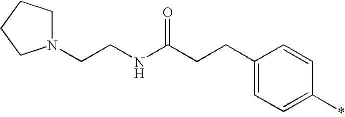 | 511.64 | 512 |
| 506 | 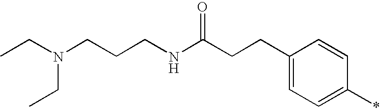 | 527.69 | 528 |
| 507 | 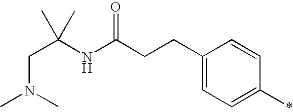 | 527.69 | 528 |
| 508 | 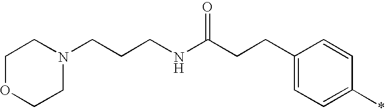 | 541.67 | 542 |
| 509 | 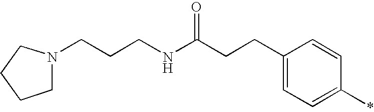 | 525.67 | 526 |
| 519 | 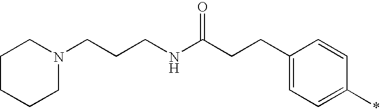 | 539.70 | 540 |
| 511 | 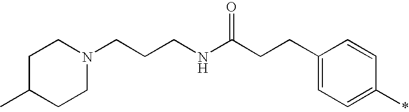 | 553.72 | 554 |
| 512 | 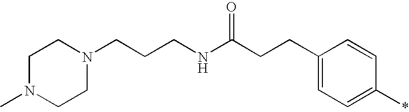 | 554.71 | 555 |
| 513 | 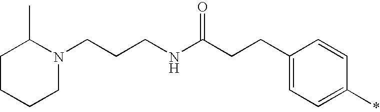 | 553.72 | 554 |
| 514 | 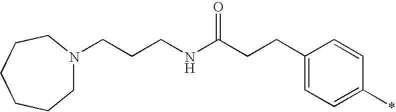 | 553.72 | 554 |
| 515 | 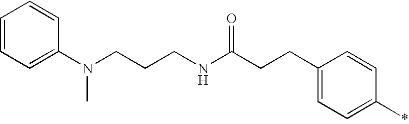 | 561.70 | 562 |
| 516 | 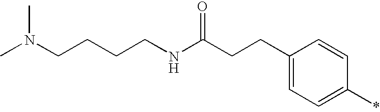 | 513.66 | 514 |
| 517 | 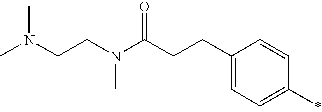 | 499.63 | 500 |
| 518 | 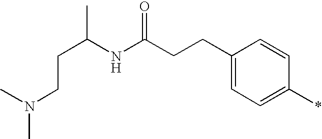 | 555.74 | 556 |
| 519 | 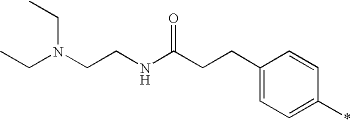 | 513.66 | 514 |
| 520 | 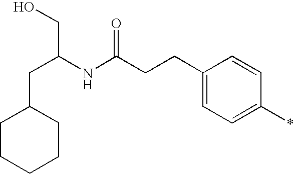 | 554.71 | 555 |
| 521 | 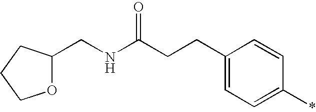 | 498.60 | 499 |
| 522 | 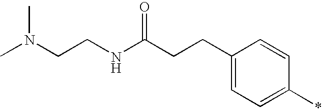 | 485.61 | 486 |
| 523 | 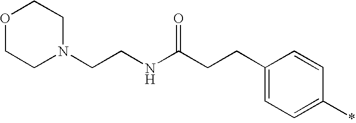 | 527.64 | 528 |
| 524 | 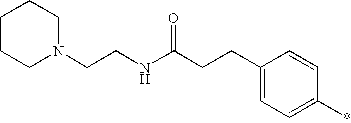 | 525.67 | 528 |
| 525 | 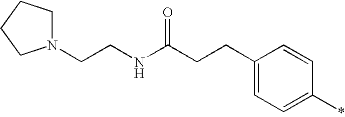 | 511.64 | 512 |
| 526 | 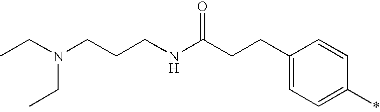 | 527.69 | 528 |
| 527 | 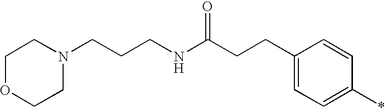 | 541.67 | 542 |
| 528 | 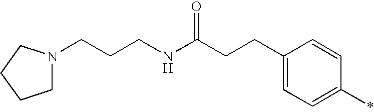 | 525.67 | 526 |
| 529 | 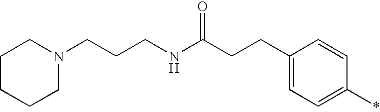 | 539.70 | 540 |
| 530 | 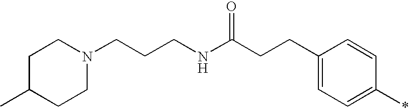 | 553.72 | 554 |
| 531 | 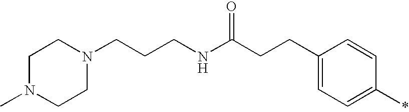 | 554.71 | 555 |
| 532 | 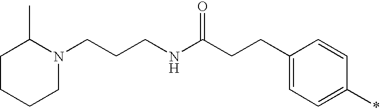 | 553.72 | 554 |
| 533 | 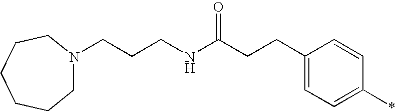 | 553.72 | 554 |
| 534 | 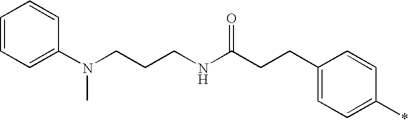 | 561.70 | 562 |
| 535 | 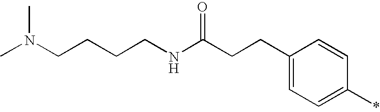 | 513.66 | 514 |
| 536 | 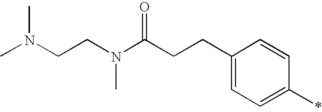 | 499.63 | 500 |
| 537 | 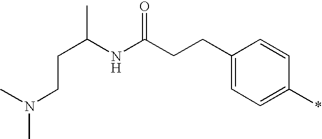 | 555.74 | 556 |
| 538 | 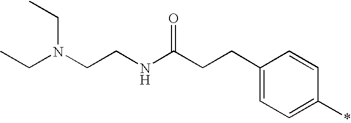 | 513.66 | 514 |
| 539 | 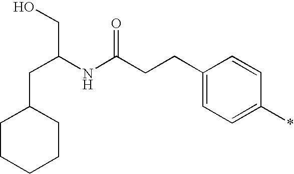 | 554.71 | 555 |
| 540 | 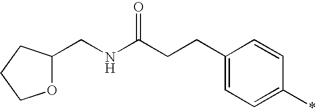 | 498.60 | 499 |
| 541 | 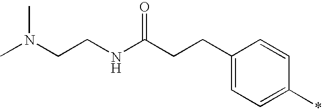 | 457.55 | 458 |
| 542 | 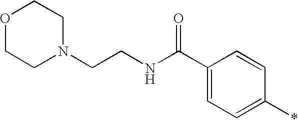 | 499.59 | 500 |
| 543 | 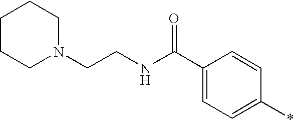 | 497.62 | 498 |
| 544 | 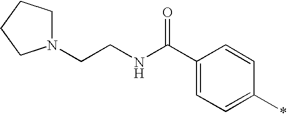 | 483.59 | 484 |
| 545 | 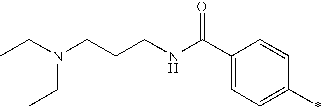 | 499.63 | 500 |
| 546 | 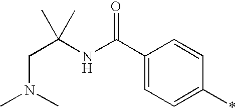 | 499.63 | 500 |
| 547 | 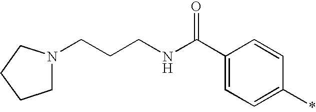 | 497.62 | 498 |
| 548 | 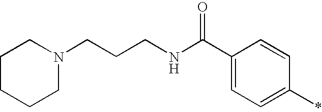 | 511.64 | 512 |
| 549 | 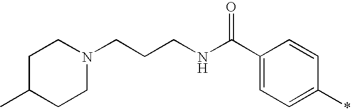 | 525.67 | 526 |
| 550 | 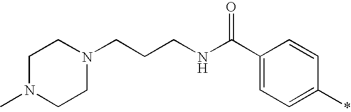 | 526.66 | 527 |
| 551 | 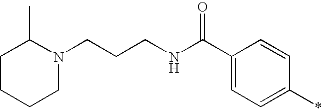 | 525.67 | 526 |
| 552 | 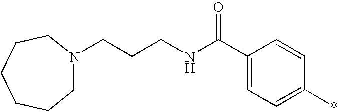 | 525.67 | 526 |
| 553 | 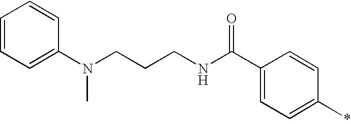 | 533.65 | 534 |
| 554 | 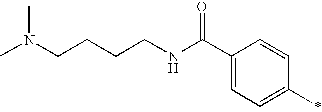 | 485.61 | 486 |
| 555 | 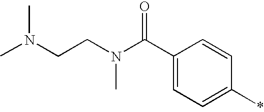 | 471.58 | 472 |
| 556 | 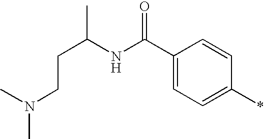 | 527.69 | 528 |
| 557 | 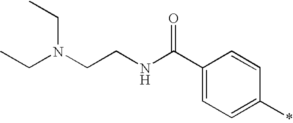 | 485.61 | 486 |
| 558 | 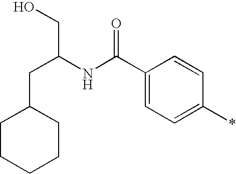 | 526.66 | 527 |
| 559 | 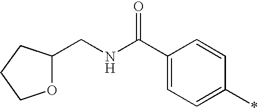 | 470.55 | 471 |
| 560 | 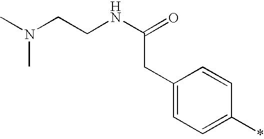 | 471.58 | 472 |
| 561 | 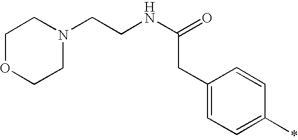 | 513.62 | 514 |
| 562 | 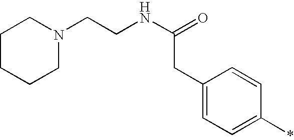 | 511.64 | 512 |
| 563 | 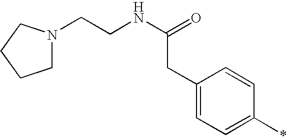 | 497.62 | 498 |
| 564 | 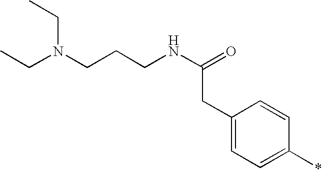 | 513.66 | 514 |
| 565 | 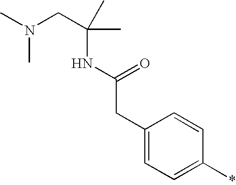 | 513.66 | 514 |
| 566 | 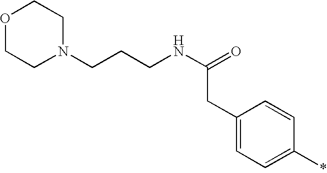 | 527.64 | 528 |
| 567 | 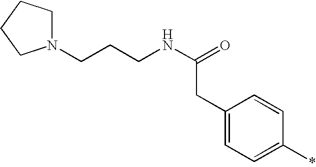 | 511.64 | 512 |
| 568 | 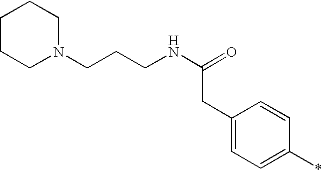 | 525.67 | 526 |
| 569 | 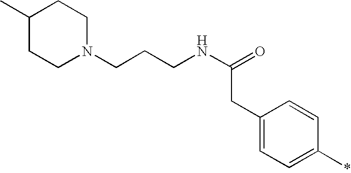 | 539.70 | 540 |
| 570 | 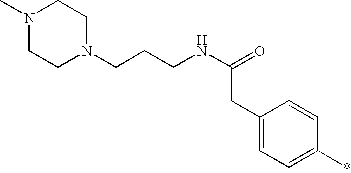 | 540.69 | 541 |
| 571 | 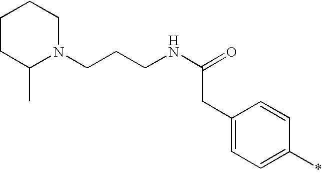 | 539.70 | 540 |
| 572 | 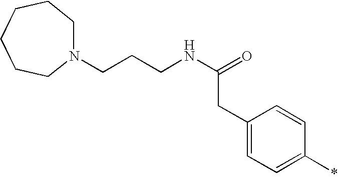 | 539.70 | 540 |
| 573 | 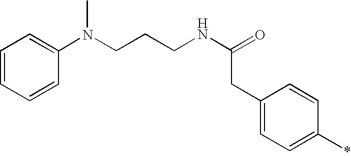 | 547.68 | 548 |
| 574 | 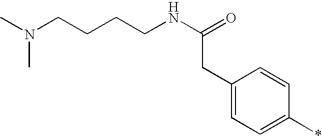 | 499.63 | 500 |
| 575 | 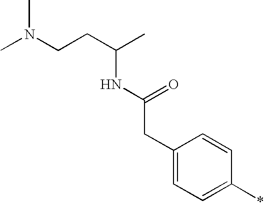 | 541.71 | 542 |
| 576 | 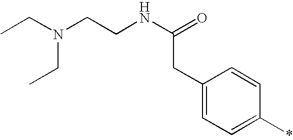 | 499.63 | 500 |
| 577 | 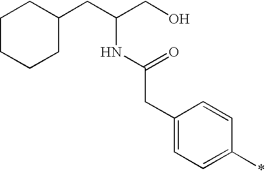 | 540.68 | 541 |
| 578 | 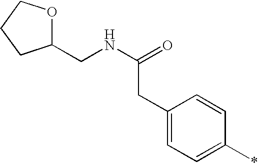 | 484.57 | 485 |
| 579 | 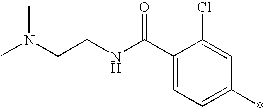 | 492.00 | 493 |
| 580 | 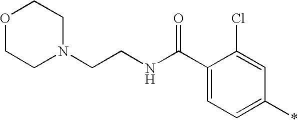 | 534.03 | 535 |
| 581 | 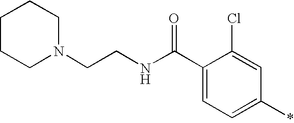 | 532.06 | 533 |
| 582 | 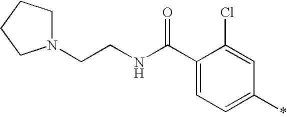 | 518.04 | 519 |
| 583 | 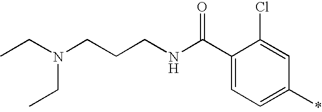 | 534.08 | 535 |
| 584 | 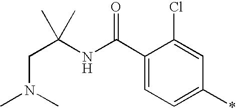 | 534.08 | 535 |
| 585 | 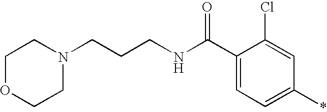 | 548.06 | 549 |
| 586 | 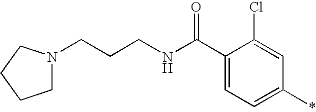 | 532.06 | 533 |
| 587 | 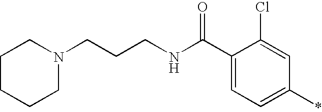 | 546.09 | 547 |
| 588 | 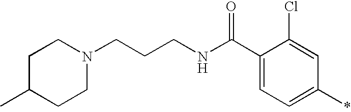 | 560.12 | 561 |
| 589 | 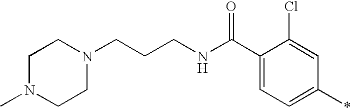 | 561.10 | 562 |
| 590 | 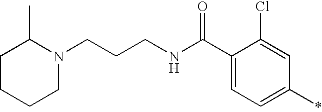 | 560.12 | 561 |
| 591 | 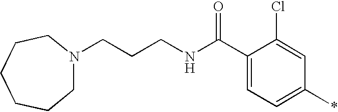 | 560.12 | 561 |
| 592 | 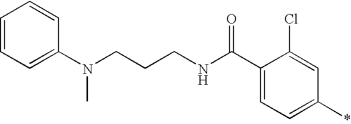 | 568.10 | 569 |
| 593 | 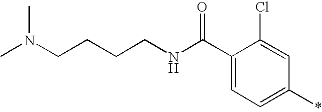 | 520.05 | 521 |
| 594 | 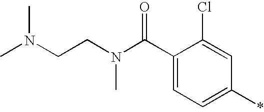 | 506.02 | 507 |
| 595 | 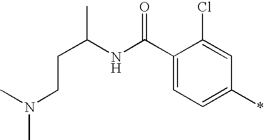 | 562.13 | 563 |
| 596 | 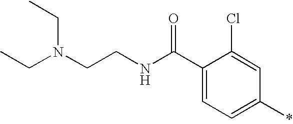 | 520.05 | 521 |
| 597 | 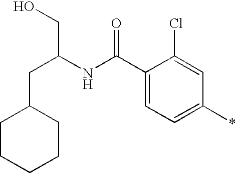 | 561.10 | 562 |
| 598 | 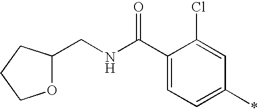 | 504.99 | 506 |
| 599 | 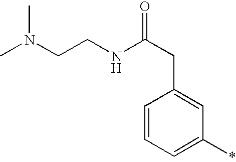 | 471.58 | 472 |
| 600 | 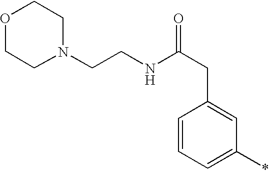 | 513.62 | 514 |
| 601 | 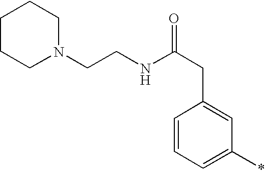 | 511.64 | 512 |
| 602 | 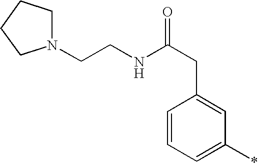 | 497.62 | 498 |
| 603 | 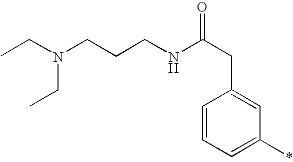 | 513.66 | 514 |
| 604 | 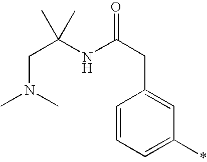 | 513.66 | 514 |
| 605 | 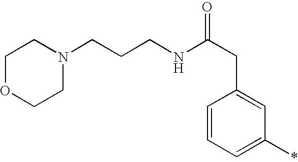 | 527.64 | 528 |
| 606 | 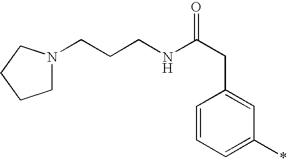 | 511.64 | 512 |
| 607 | 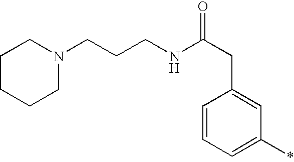 | 525.67 | 526 |
| 608 | 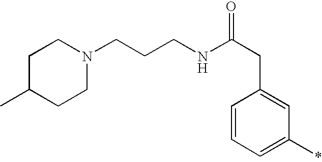 | 539.70 | 540 |
| 609 | 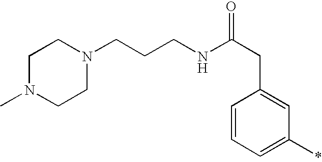 | 540.69 | 541 |
| 610 | 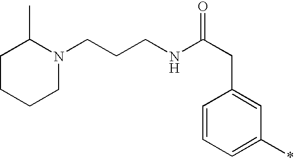 | 539.70 | 540 |
| 611 | 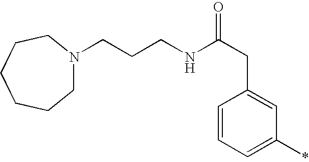 | 539.70 | 540 |
| 612 | 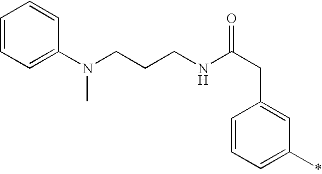 | 547.68 | 548 |
| 613 | 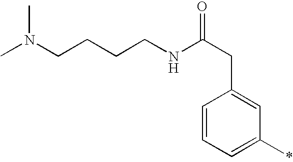 | 499.63 | 500 |
| 614 | 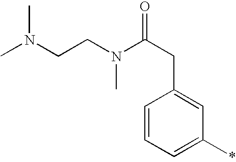 | 485.61 | 486 |
| 615 | 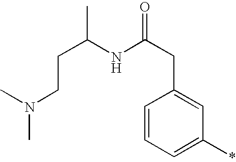 | 541.71 | 542 |
| 616 | 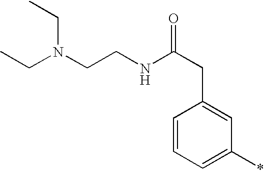 | 499.63 | 500 |
| 617 | 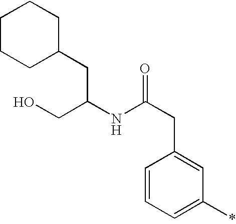 | 540.68 | 541 |
| 618 | 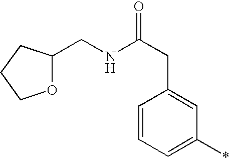 | 484.57 | 485 |
| 619 | 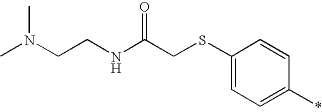 | 503.65 | 504 |
| 620 | 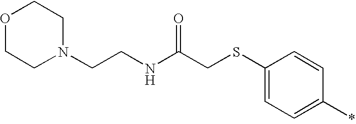 | 545.68 | 546 |
| 621 | 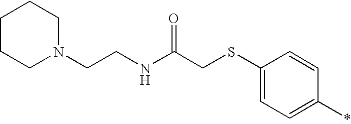 | 543.71 | 544 |
| 622 | 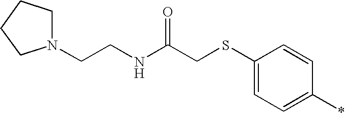 | 529.68 | 530 |
| 623 | 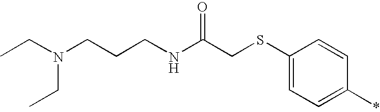 | 545.73 | 546 |
| 624 | 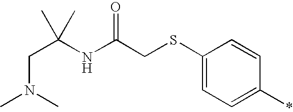 | 545.73 | 546 |
| 625 | 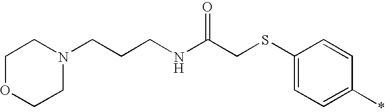 | 559.71 | 560 |
| 626 | 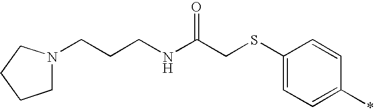 | 543.71 | 544 |
| 627 | 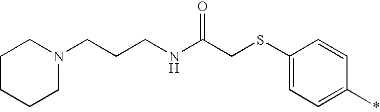 | 557.74 | 558 |
| 628 | 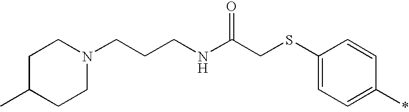 | 571.76 | 572 |
| 629 | 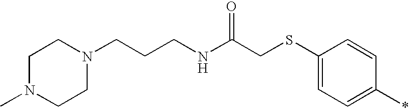 | 572.75 | 573 |
| 630 | 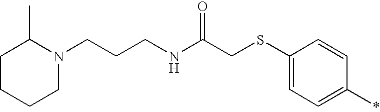 | 571.76 | 572 |
| 631 | 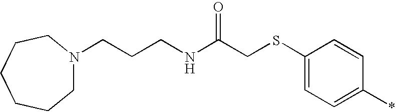 | 571.76 | 572 |
| 632 | 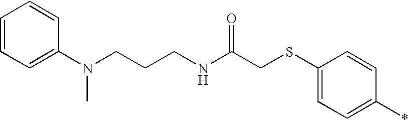 | 579.74 | 580 |
| 633 | 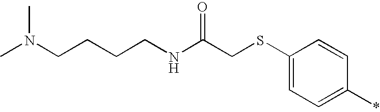 | 531.70 | 532 |
| 634 | 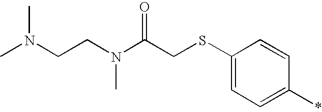 | 517.67 | 518 |
| 635 | 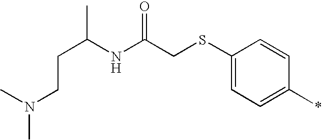 | 573.78 | 574 |
| 636 | 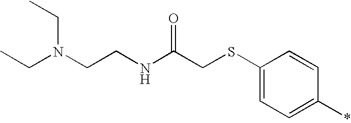 | 531.70 | 532 |
| 637 | 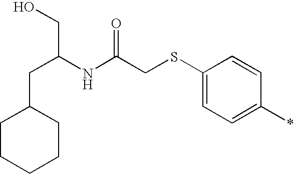 | 572.75 | 573 |
| 638 | 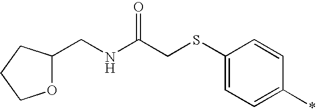 | 516.64 | 517 |
| 639 | 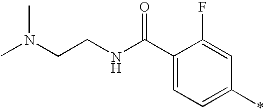 | 475.54 | 476 |
| 640 | 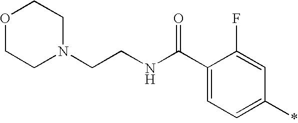 | 517.58 | 518 |
| 641 | 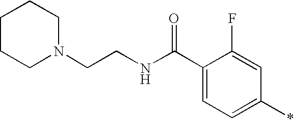 | 515.61 | 516 |
| 642 | 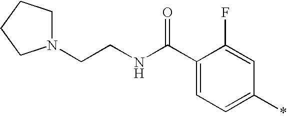 | 501.58 | 502 |
| 643 | 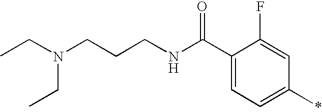 | 517.62 | 518 |
| 644 | 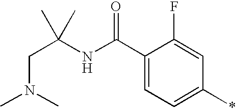 | 517.62 | 518 |
| 645 | 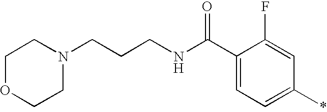 | 531.61 | 532 |
| 646 | 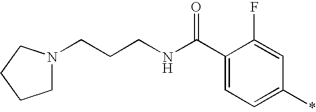 | 515.61 | 516 |
| 647 | 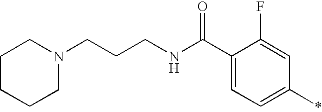 | 529.63 | 530 |
| 648 | 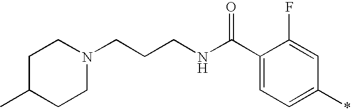 | 543.66 | 544 |
| 649 | 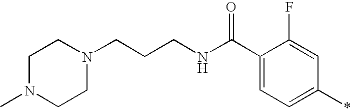 | 544.65 | 545 |
| 650 | 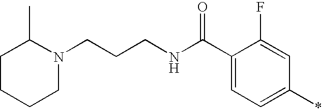 | 543.66 | 544 |
| 651 | 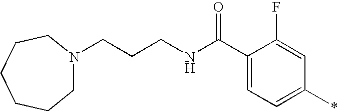 | 543.66 | 544 |
| 652 | 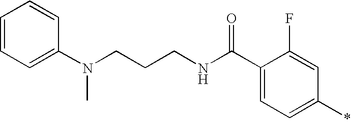 | 551.64 | 552 |
| 653 | 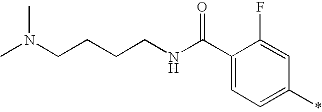 | 503.60 | 504 |
| 654 | 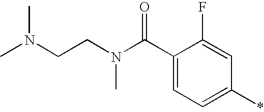 | 489.57 | 490 |
| 655 | 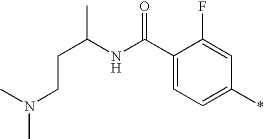 | 545.68 | 546 |
| 656 | 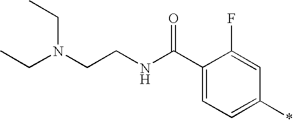 | 503.60 | 504 |
| 657 | 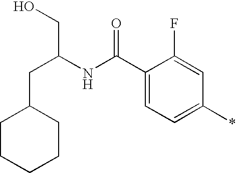 | 544.65 | 545 |
| 658 | 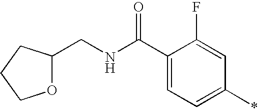 | 488.54 | 489 |
| 659 | 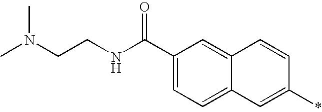 | 507.61 | 508 |
| 660 | 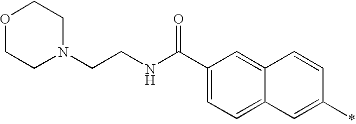 | 549.65 | 550 |
| 661 | 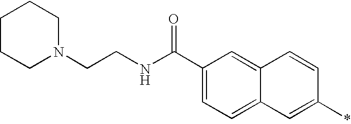 | 547.68 | 548 |
| 662 | 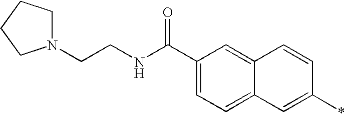 | 533.65 | 534 |
| 663 | 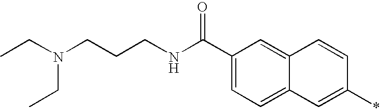 | 549.69 | 550 |
| 664 | 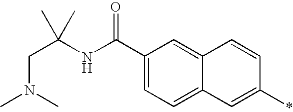 | 549.69 | 550 |
| 665 | 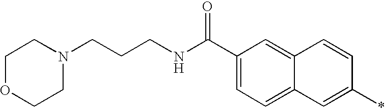 | 563.68 | 564 |
| 666 | 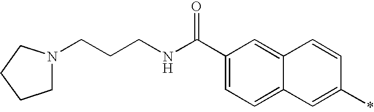 | 547.68 | 548 |
| 667 | 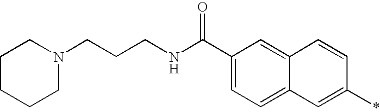 | 561.70 | 562 |
| 668 | 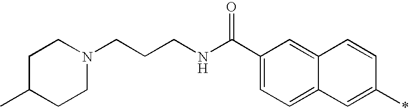 | 575.73 | 576 |
| 669 | 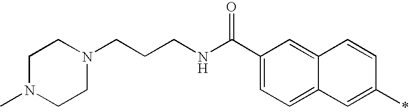 | 576.72 | 577 |
| 670 | 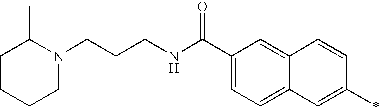 | 575.73 | 576 |
| 671 | 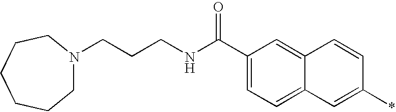 | 575.73 | 576 |
| 672 | 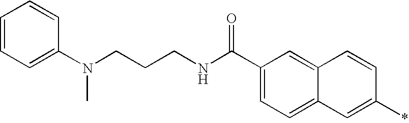 | 583.71 | 584 |
| 673 | 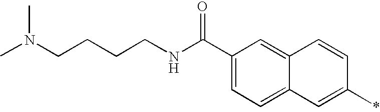 | 535.67 | 536 |
| 674 | 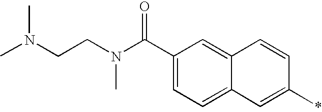 | 521.64 | 522 |
| 675 | 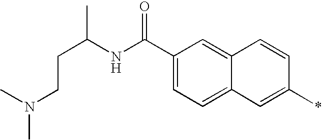 | 577.75 | 578 |
| 676 | 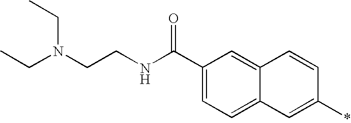 | 535.67 | 536 |
| 677 | 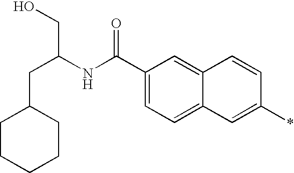 | 576.71 | 577 |
| 678 | 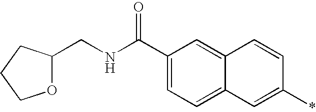 | 520.61 | 521 |
| 679 | 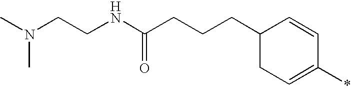 | 499.63 | 500 |
| 680 | 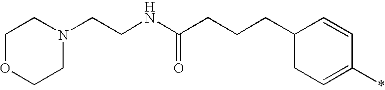 | 541.67 | 542 |
| 681 | 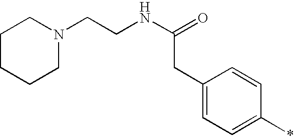 | 539.70 | 540 |
| 682 | 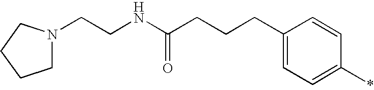 | 525.67 | 526 |
| 683 | 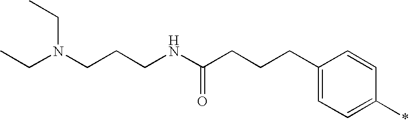 | 541.71 | 542 |
| 684 | 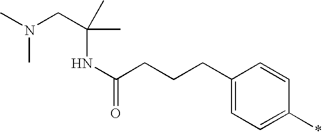 | 541.71 | 542 |
| 685 | 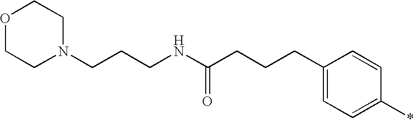 | 555.70 | 556 |
| 686 | 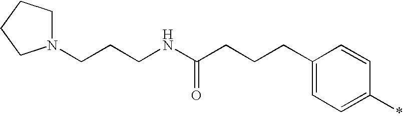 | 539.70 | 540 |
| 687 | 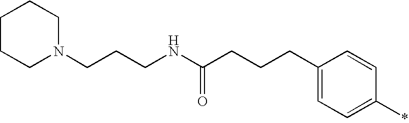 | 553.72 | 554 |
| 688 | 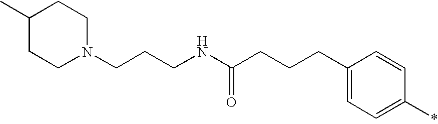 | 567.75 | 568 |
| 689 | 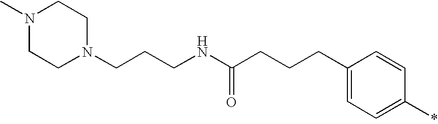 | 568.74 | 569 |
| 690 | 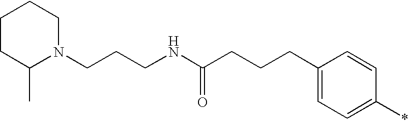 | 567.75 | 568 |
| 691 | 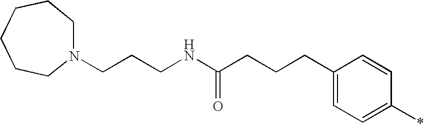 | 567.75 | 568 |
| 692 | 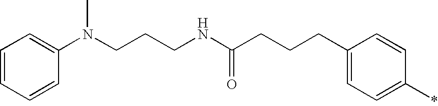 | 575.73 | 576 |
| 693 | 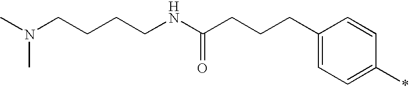 | 527.69 | 528 |
| 694 | 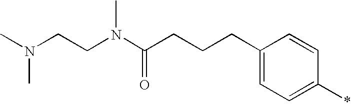 | 513.66 | 514 |
| 695 | 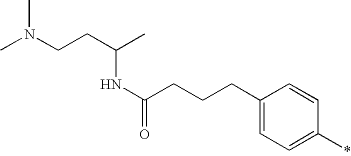 | 569.77 | 570 |
| 696 | 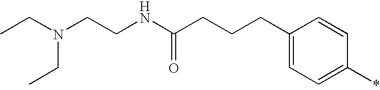 | 527.69 | 528 |
| 697 | 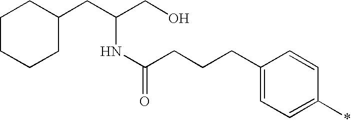 | 568.74 | 569 |
| 698 | 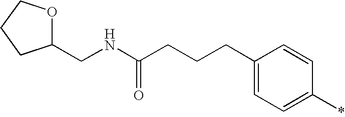 | 512.63 | 513 |

### EXAMPLE 699

### (E or Z)-Cyano-{3-ethyl-5-(E/Z)-[(7-hydroxy-naphthalen-1-ylamino)-methylene]-4-oxo-thiazolidin-2-ylidene}-acetic acid ethyl ester

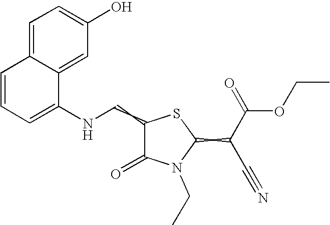

Analogously to Example 1, process variant B, 91.8 mg of product is obtained from 98 mg of the substance that is described under Example c) and 52.5 mg of 7-hydroxy-1-naphthylamine.

¹H-NMR (DMSO-d6, stored with K₂CO₃, main isomer): δ=1.13-1.35 (6H), 4.08-4.39 (4H), 7.16 (1H), 7.23-7.38 (3H), 7.73 (1H), 7.84 (1H), 8.05 (1H), 9.99 (1H), 10.57 (1H) ppm.

### EXAMPLE 700

### (E or Z)-Cyano-{3-ethyl-5-(E/Z)-[(5-hydroxy-naphthalen-2-ylamino)-methylene]-4-oxo-thiazolidin-2-ylidene}-acetic acid ethyl ester

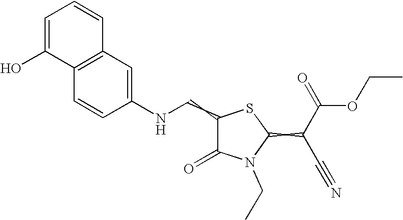

Analogously to Example 1, process variant B, 111 mg of product is obtained from 98 mg of the substance that is described under Example c) and 47.8 mg of 5-hydroxy-2-naphthylamine.

¹H-NMR (DMSO-d6, stored with K₂CO₃, main isomer): δ=1.26 (3H), 1.27 (3H), 4.18-4.34 (4H), 6.76 (1H), 7.22-7.35 (2H), 7.44 (1H), 7.70 (1H), 8.10 (1H), 8.36 (1H), 10.11 (1H), 10.70 (1H) ppm.

### EXAMPLE 701

### (E or Z)-(5-(E/Z)-{[4-(2-Carboxy-ethylcarbamoyl)-phenylamino]-methylene}-3-ethyl-4-oxo-thiazolidin-2-ylidene)-cyanoacetic acid ethyl ester

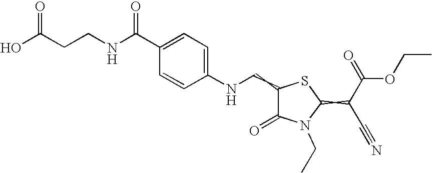

Analogously to Example 1, process variant B, 111 mg of product is obtained from 98 mg of the substance that is described under Example c) and 68.7 mg of 3-(4-amino-benzoylamino)-propionic acid.

¹H-NMR (DMSO-d6, stored with K₂CO₃, main isomer): δ=1.24 (3H), 1.27 (3H), 2.46-2.54 (2H), 3.38-3.50 (2H), 4.18-4.31 (4H), 7.37 (2H), 7.83 (2H), 8.27 (1H), 8.46 (1H), 10.6 (broad, 2H) ppm.

### EXAMPLE 702

### (E or Z)-{5-(E/Z)-[(4-Carboxymethylsulfanyl-phenylamino)-methylene]-3-ethyl-4-oxo-thiazolidin-2-ylidene}-cyanoacetic acid ethyl ester

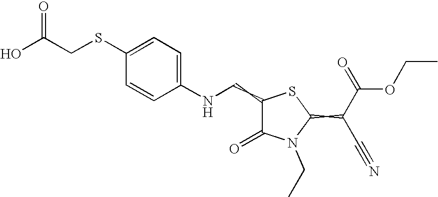

Analogously to Example 1, process variant B, 112 mg of product is obtained from 98 mg of the substance that is described under Example c) and 60.5 mg of (4-amino-phenylsulfanyl)-ethanoic acid.

¹H-NMR (DMSO-d6, stored with K₂CO₃, main isomer): δ=1.24 (3H), 1.27 (3H), 3.74 (2H), 4.16-4.32 (4H), 7.25-7.41 (4H), 8.18 (1H), 10.54 (1H), 12.74 (1H) ppm.

### EXAMPLE 703

### (E or Z)-Cyano-{3-ethyl-5-(E/Z)-[(1H-indol-6-ylamino)-methylene]-4-oxo-thiazolidin-2-ylidene}-acetic acid ethyl ester

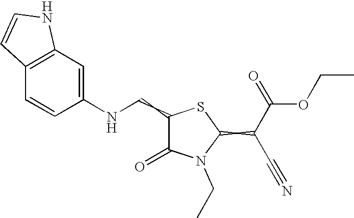

Analogously to Example 1, process variant B, 81.6 mg of product is obtained from 98 mg of the substance that is described under Example c) and 43.6 mg of 1H-indol-6-ylamine.

¹H-NMR (DMSO-d6, stored with K₂CO₃, main isomer): δ=1.24 (3H), 1.26 (3H), 4.15-4.32 (4H), 6.42 (1H), 7.08 (1H), 7.33-7.43 (2H), 7.47 (1H), 8.19 (1H), 10.59 (1H), 11.14 (1H) ppm.

### EXAMPLE 704

### (E or Z)-Cyano-{3-ethyl-5-(E/Z)-[(3-hydroxy-4-methyl-phenylamino)-methylene]-4-oxo-thiazolidin-2-ylidene}-acetic acid ethyl ester

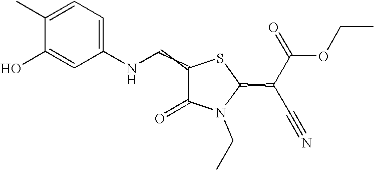

Analogously to Example 1, process variant B, 89.9 mg of product is obtained from 98 mg of the substance that is described under Example c) and 40.6 mg of 5-amino-2-methyl-phenol.

¹H-NMR (DMSO-d6, stored with K₂CO₃, main isomer): δ=1.24 (3H), 1.26 (3H), 2.07 (3H), 4.16-4.29 (4H), 6.66 (1H), 6.71 (1H), 7.03 (1H), 8.04 (1H), 9.56 (1H), 10.49 (1H) ppm.

### EXAMPLE 705

### (E or Z)-Cyano-{3-ethyl-5-(E/Z)-[(3-hydroxy-4-methoxy-phenylamino)-methylene]-4-oxo-thiazolidin-2-ylidene}-acetic acid ethyl ester

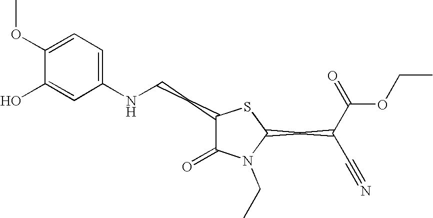

Analogously to Example 1, process variant B, 88.0 g of product is obtained from 98 mg of the substance that is described under Example c) and 46.0 mg of 5-amino-2-methoxy-phenol.

¹H-NMR (DMSO-d6, stored with K₂CO₃, main isomer): δ=1.24 (3H), 1.26 (3H), 3.75 (3H), 4.16-4.30 (4H), 6.67-6.79 (2H), 6.90 (1H), 8.02 (1H), 9.31 (1H), 10.42 (1H) ppm.

### EXAMPLE 706

### (E or Z)-{5-(E/Z)-[(4-Bromo-phenylamino)-methylene]-3-ethyl-4-oxo-thiazolidin-2-ylidene}-cyanoacetic acid ethyl ester

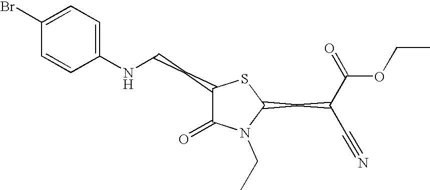

Analogously to Example 1, process variant B, 90.7 mg of product is obtained from 98 mg of the substance that is described under Example c) and 56.8 mg of 4-bromo-aniline.

¹H-NMR (DMSO-d6, stored with K₂CO₃, main isomer): δ=1.24 (3H), 1.26 (3H), 4.17-4.31 (4H), 7.29 (2H), 7.52 (2H), 8.18 (1H), 10.55 (1H) ppm.

### EXAMPLE 707

### (E or Z)-[Cyano-[3-ethyl-4-oxo-5-(E/Z)-(phthalazin-5-ylaminomethylene)-thiazolidin-2-ylidene]-acetic acid ethyl ester

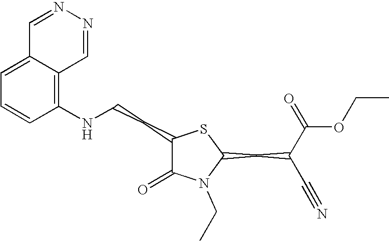

Analogously to Example 1, process variant B, 172 mg of product is obtained from 196 mg of the substance that is described under Example c) and 106 mg of phthalazin-5-ylamine.

¹H-NMR (DMSO-d6, stored with K₂CO₃, main isomer): δ=1.26 (3H), 1.27 (3H), 4.17-4.35 (4H), 7.84-8.06 (3H), 8.21 (1H), 9.68 (1H), 9.94 (1H), 10.89 (1H) ppm.

### EXAMPLE 708

### (E or Z)-[Cyano-{3-ethyl-5-[(2-methyl-1,3-dioxo-2,3-dihydro-1H-isoindol-5-(E/Z)-ylamino)-methylene]-4-oxo-thiazolidin-2-ylidene}-acetic acid ethyl ester

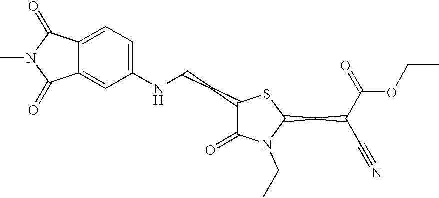

Analogously to Example 1, process variant B, 108 mg of product is obtained from 98.0 mg of the substance that is described under Example c) and 58.0 mg of 4-amino-N-methylphthalimide.

¹H-NMR (DMSO-d6, stored with K₂CO₃, main isomer): δ=1.26 (3H), 1.28 (3H), 3.05 (3H), 4.16-4.37 (4H), 7.67 (1H), 7.72 (1H), 7.79 (1H), 8.29 (1H), 10.57 (1H) ppm.

### EXAMPLE 709

### (E or Z)-[Cyano-{3-ethyl-5-(E/Z)-[(5-methyl-1H-[1,2,4]triazol-3-ylamino)-methylene]-4-oxo-thiazolidin-2-ylidene}-acetic acid ethyl ester

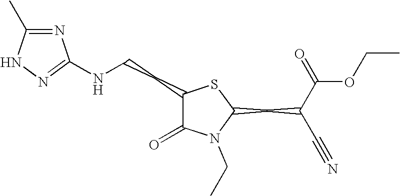

Analogously to Example 1, process variant B, 95.0 mg of product is obtained from 98.0 mg of the substance that is described under Example c) and 32.4 mg of 3-amino-5-methyl-1,2,4-triazole.

¹H-NMR (DMSO-d6, stored with K₂CO₃, main isomer): δ=1.23 (3H), 1.26 (3H), 2.33 (3H), 4.23 (4H), 8.30 (1H), 11.31 (1H), 13.39 (1H) ppm.

### EXAMPLE 710

### (E or Z)-[Cyano-{3-ethyl-5-(E/Z)-[(1H-indazol-5-ylamino)-methylene]-4-oxo-thiazolidin-2-ylidene}-acetic acid ethyl ester

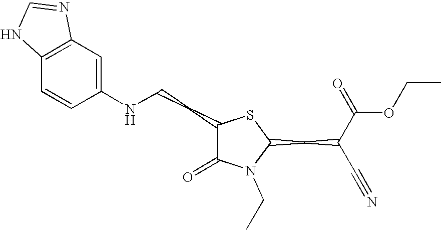

Analogously to Example 1, process variant B, 101 mg of product is obtained from 98.0 mg of the substance that is described under Example c) and 43.9 mg of 5-aminoindazole.

¹H-NMR (DMSO-d6, stored with K₂CO₃, main isomer): δ=1.25 (3H), 1.26 (3H), 4.23 (2H), 4.25 (2H), 7.37 (1H), 7.55 (1H), 7.68 (1H), 8.04 (1H), 8.23 (1H), 10.62 (1H), 13.09 (1H) ppm.

### EXAMPLE 711

### (E or Z)-[Cyano-{3-ethyl-5-(E/Z)-[(1H-indazol-7-ylamino)-methylene]-4-oxo-thiazolidin-2-ylidene}-acetic acid ethyl ester

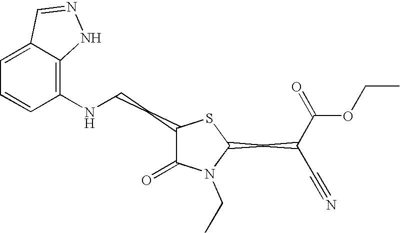

Analogously to Example 1, process variant B, 64.0 mg of product is obtained from 148.2 mg of the substance that is described under Example c) and 146.5 mg of 7-aminoindazole.

¹H-NMR (DMSO-d6, stored with K₂CO₃, main isomer): δ=1.25 (3H), 1.26 (3H), 4.14-4.35 (414), 6.99-7.18 (114), 7.31 (1H), 7.44-7.63 (114), 8.07-8.30 (2H), 10.20 (1H), 13.04 (1H) ppm.

### EXAMPLE 712

### (E or Z)-Cyano-{3-ethyl-4-oxo-5-(E/Z)-[(1-oxo-2,3-dihydro-1H-isoindol-4-ylamino)-methylene]-thiazolidin-2-ylidene}-acetic acid ethyl ester

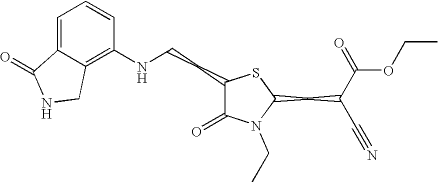

Analogously to Example 1, process variant B, 214 mg of product is obtained from 101 mg of the substance that is described under Example c) and 200 mg of 4-amino-2,3-dihydro-isoindol-1-one.

¹H-NMR (DMSO-d6, stored with K₂CO₃, main isomer): δ=1.15 (3H), 1.17 (3H), 4.04-4.22 (4H), 4.38 (2H), 7.31-7.44 (3H), 8.07 (1H), 8.56 (1H), 10.26 (1H) ppm.

### EXAMPLE 713

### (E or Z)-Cyano-{3-ethyl-4-oxo-5-(E/Z)-[(1-oxo-1,2-dihydro-isoquinolin-5-ylamino)-methylene]-thiazolidin-2-ylidene}-acetic acid ethyl ester

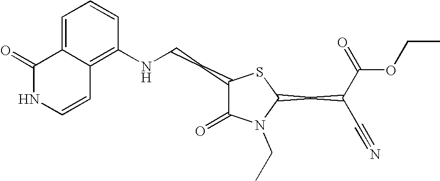

Analogously to Example 1, process variant B, 284 mg of product is obtained from 111 mg of the substance that is described under Example c) and 204 mg of 5-amino-2H-isoquinolin-1-one.

¹H-NMR (DMSO-d6, stored with K₂CO₃, main isomer): δ=1.23 (3H), 1.25 (3H), 4.13-4.30 (4H), 6.74 (1H), 7.26 (1H), 7.43-7.63 (2H), 8.00-8.11 (2H), 10.50 (1H), 11.41 (1H) ppm.

### EXAMPLE 714

### (E or Z)-[[5-(E/Z)-({4-[2-(4-Amino-phenyl)-ethyl]-phenylamino}-methylene)-3-ethyl-4-oxo-thiazolidin-2-ylidene]-cyanoacetic acid ethyl ester

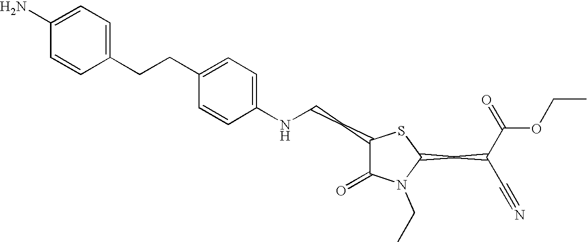

Analogously to Example 1, process variant B, 178 mg of product is obtained from 296 mg of the substance that is described under Example c) and 212 mg of 4,4′-ethylenedianiline.

¹H-NMR (DMSO-d6, stored with K₂CO₃, main isomer): δ=1.24 (3H), 1.26 (3H), 2.71 (4H), 4.14-4.32 (4H), 4.82 (2H), 6.47 (2H), 6.85 (2H), 7.10-7.25 (4H), 8.18 (1H), 10.51 (1H) ppm.

### EXAMPLE 715

### (E or Z)-[(5-(E/Z)-{[4-(4-Amino-benzyl)-phenylamino]-methylene}-3-ethyl-4-oxo-thiazolidin-2-ylidene)-cyanoacetic acid ethyl ester

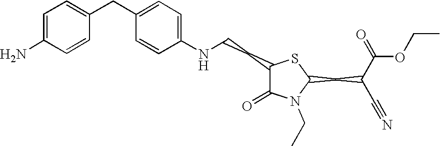

Analogously to Example 1, process variant B, 1.24 g of product is obtained from 980 mg of the substance that is described under Example c) and 654 g of bis-(4-aminophenyl)-methane.

¹H-NMR (DMSO-d6, stored with K₂CO₃, main isomer): δ=1.24 (3H), 1.27 (3H), 3.70 (2H), 4.15-4.30 (4H), 4.88 (2H), 6.49 (2H), 6.86 (2H), 7.16 (2H), 7.24 (2H), 8.15 (1H), 10.52 (1H) ppm.

### EXAMPLE 716

### (E or Z)-[Cyano-[3-ethyl-5-(E/Z)-({4-[4-(3-ethyl-thioureido)-benzyl]-phenylamino}-methylene)-4-oxo-thiazolidin-2-ylidene]-acetic acid ethyl ester

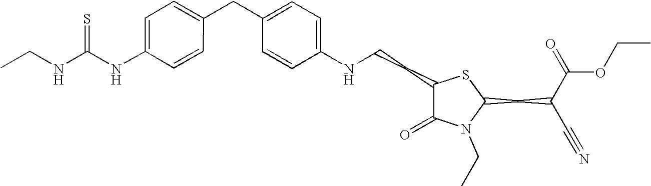

17.5 μl of ethyl isothiocyanate is added to a solution of 89.7 mg of the compound, produced in Example 715, in 0.1 ml of DMSO, and it is stirred for 18 hours at 25° C. Then, it is mixed with 8 ml of ethanol, heated to 50° C., filtered on a G4-frit and rewashed with ethanol. After drying in a vacuum, 66.0 mg of the desired product is obtained.

¹H-NMR (DMSO-d6, stored with K₂CO₃, main isomer): δ=1.09 (3H), 1.24 (3H), 1.26 (3H), 3.46 (2H), 3.87 (2H), 4.15-4.30 (4H), 7.08-7.34 (8H), 7.66 (1H), 8.17 (1H), 9.36 (1H), 10.52 (1H) ppm.

### EXAMPLE 717

### (E or Z)-[Cyano-[3-ethyl-4-oxo-5-(E/Z)-({4-[4-(3-phenyl-ureido)-benzyl]-phenylamino}-methylene)-thiazolidin-2-ylidene]-acetic acid ethyl ester

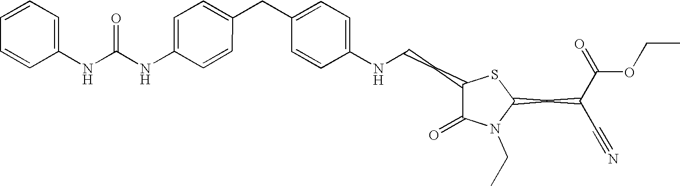

Analogously to Example 716, 92.0 mg of product is obtained from 89.7 mg of the substance that is described under Example 715 and 21.7 μl of phenyl isocyanate.

¹H-NMR (DMSO-d6, stored with K₂CO₃, main isomer): δ=1.24 (3H), 1.26 (3H), 3.85 (2H), 4.16-4.30 (4H), 6.95 (1H), 7.13 (2H), 7.17-7.32 (6H), 7.36 (2H), 7.43 (2H), 8.17 (1H), 8.59 (2H), 10.53 (1H) ppm.

### EXAMPLE 718

### (E or Z)-[Cyano-[3-ethyl-5-(E/Z)-({4-[4-(3-methoxymethyl-ureido)-benzyl]-phenylamino}-methylene)-4-oxo-thiazolidin-2-ylidene]-acetic acid ethyl ester

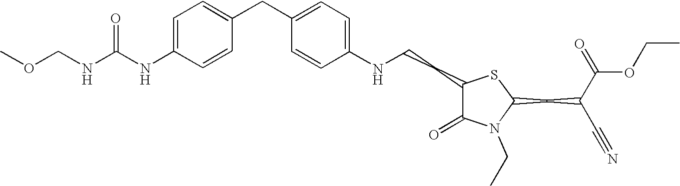

Analogously to Example 716, 85.0 mg of product is obtained from 89.7 mg of the substance that is described under Example 715 and 17.4 μl of methoxymethyl isocyanate.

¹H-NMR (DMSO-d6, stored with K₂CO₃, main isomer): δ=1.24 (3H), 1.26 (3H), 3.18 (3H), 3.82 (2H), 4.16-4.29 (4H), 4.50 (2H), 6.91 (1H), 7.09 (2H), 7.18 (2H), 7.24 (2H), 7.32 (2H), 8.16 (1H), 8.56 (1H), 10.52 (1H) ppm.

### EXAMPLE 719

### (E or Z)-[Cyano-[3-ethyl-4-oxo-5-(E/Z)-({4-[4-(3-phenyl-thioureido)-benzyl]-phenylamino}-methylene)-thiazolidin-2-ylidene]-acetic acid ethyl ester

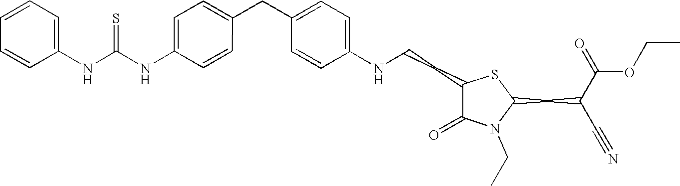

Analogously to Example 716, 91.0 mg of product is obtained from 89.7 mg of the substance that is described under Example 715 and 24.0 μl of phenyl isothiocyanate.

¹H-NMR (DMSO-d6, stored with K₂CO₃, main isomer): δ=1.24 (3H), 1.26 (3H), 3.88 (2H), 4.17-4.30 (4H), 7.14 (1H), 7.15-7.41 (9H), 7.46 (2H), 8.17 (1H), 9.73 (2H), 10.53 (1H) ppm.

### EXAMPLE 720

### (E or Z)-[Cyano-[5-(E/Z)-({4-[4-(3-ethoxycarbonylmethyl-ureido)-benzyl]-phenylamino}-methylene)-3-ethyl-4-oxo-thiazolidin-2-ylidene]-acetic acid ethyl ester

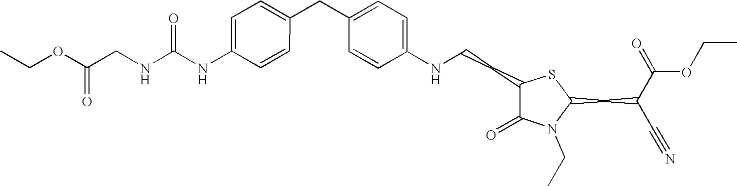

Analogously to Example 716, 106 mg of product is obtained from 89.7 mg of the substance that is described under Example 715 and 23.0 μl of isocyanatoacetic acid ethyl ester.

¹H-NMR (DMSO-d6, stored with K₂CO₃, main isomer): δ=1.24 (6H), 1.26 (3H), 3.78-3.89 (4H), 4.10 (2H), 4.17-4.30 (4H), 6.39 (1H), 7.07 (2H), 7.18 (2H), 7.24 (2H), 7.30 (2H), 8.17 (1H), 8.71 (1H), 10.51 (1H) ppm.

### EXAMPLE 721

### (E or Z)-[2-Cyano-2-(3-ethyl-4-oxo-5-(E/Z)-phenylaminomethylene-thiazolidin-2-ylidene)-acetylamino]-acetic acid ethyl ester

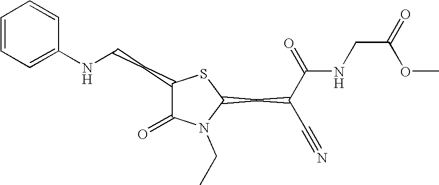

60.0 mg of the acid that is produced in Reference Example x is dissolved in 0.75 ml of dimethylformamide and stirred with 67.1 mg of TBTU and 21.1 mg of triethylamine for 30 minutes at 25° C. Then, 26.2 mg of glycine methyl ester hydrochloride is added, and it is stirred for 20 hours at 25° C. It is diluted with 200 ml of ethyl acetate, washed once with 20 ml of saturated sodium bicarbonate solution and once with 20 ml of saturated sodium chloride solution. After drying on sodium sulfate and filtration, it is concentrated by evaporation in a vacuum. The crude product that is obtained is purified by thick-layer chromatography with hexane/ethyl acetate 1:1. In this way, 25.1 mg of the desired product is obtained.

¹H-NMR (DMSO-d6, stored with K₂CO₃, main isomer): δ=1.26 (3H), 3.65 (3H), 3.91 (2H), 4.24 (2H), 7.07 (1H), 7.26-7.40 (4H), 8.06 (1H), 8.12 (1H), 10.34 (1H) ppm.

### EXAMPLE 722

### (E or Z)-[2-Cyano-2-(3-ethyl-4-oxo-5-(E/Z)-phenylaminomethylene-thiazolidin-2-ylidene)-N-pyridin-3-ylmethyl-acetamide

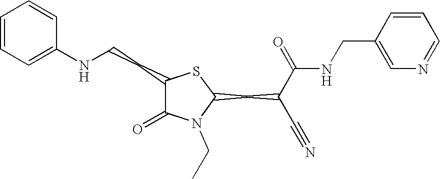

Analogously to Example 721, 47.3 mg of product is obtained from 60 mg of the acid that is described under Example xx) and 22.6 mg of 3-picolylamine.

¹H-NMR (DMSO-d6, stored with K₂CO₃, main isomer): δ=1.25 (3H), 4.23 (2H), 4.38 (2H), 7.07 (1H), 7.24-7.39 (4H), 7.43 (1H), 7.80 (1H), 8.09 (1H), 8.43 (1H), 8.49 (1H), 8.58 (1H), 10.29 (1H) ppm.

### EXAMPLE 723

### (E or Z)-[2-Cyano-2-(3-ethyl-4-oxo-5-(E/Z)-phenylaminomethylene-thiazolidin-2-ylidene)-N-(3-imidazol-1-yl-propyl)-acetamide

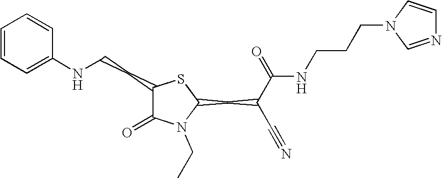

Analogously to Example 721, 34.1 mg of product is obtained from 60 mg of the acid that is described under Example xx) and 26.2 mg of 1-(3-aminopropyl)-imidazole.

¹H-NMR (DMSO-d6, stored with K₂CO₃, main isomer): δ=1.25 (3H), 1.93 (2H), 3.17 (2H), 3.97 (2H), 4.23 (2H), 6.90 (1H), 7.05 (1H), 7.20 (1H), 7.24-7.39 (4H), 7.66 (1H), 7.78 (1H), 8.11 (1H), 10.31 (1H) ppm.

### EXAMPLE 724

### (E or Z)-[2-Cyano-2-(3-ethyl-4-oxo-5-(E/Z)-phenylaminomethylene-thiazolidin-2-ylidene)-N-(4-fluoro-benzyl)-acetamide

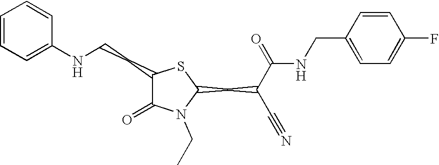

Analogously to Example 721, 122.3 mg of product is obtained from 100 mg of the acid that is described under Example xx) and 43.6 mg of 4-fluorobenzylamine.

¹H-NMR (DMSO-d6, stored with K₂CO₃, main isomer): δ=1.24 (3H), 4.23 (2H), 4.32 (2H), 7.06 (1H), 7.15 (2H), 7.25-7.42 (6H), 8.09 (1H), 8.34 (1H), 10.29 (1H) ppm.

### EXAMPLE 725

### (E or Z)-[2-Cyano-2-(3-ethyl-4-oxo-5-(E/Z)-phenylaminomethylene-thiazolidin-2-ylidene)-N-(3-morpholin-4-yl-propyl)-acetamide

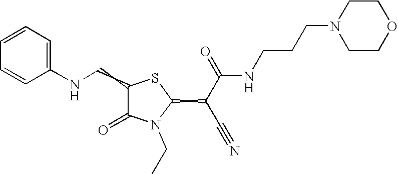

Analogously to Example 721, 34.9 mg of product is obtained from 60 mg of the acid that is described under Example xx) and 30.1 mg of 4-(3-aminopropyl)-morpholine.

¹H-NMR (DMSO-d6, stored with K₂CO₃, main isomer): δ=1.24 (3H), 1.64 (2H), 2.27-2.39 (6H), 3.25 (2H), 3.61 (4H), 4.22 (2H), 7.05 (1H), 7.22-7.39 (4H), 7.76 (1H), 8.10 (1H), 10.30 (1H) ppm.

### EXAMPLE 726

### (E or Z)-[2-Cyano-2-(3-ethyl-4-oxo-5-(E/Z)-phenylaminomethylene-thiazolidin-2-ylidene)-N-(2-morpholin-4-yl-ethyl)-acetamide

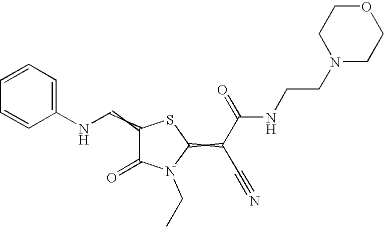

Analogously to Example 721, 37.2 mg of product is obtained from 60 mg of the acid that is described under Example xx) and 37.2 mg of 4-(2-aminoethyl)-morpholine.

¹H-NMR (DMSO-d6, stored with K₂CO₃, main isomer): δ=1.24 (3H), 2.35-2.47 (6H), 3.30 (2H), 3.57 (4H), 4.22 (2H), 7.06 (1H), 7.24-7.40 (4H), 7.54 (1H), 8.10 (1H), 10.31 (1H) ppm.

### EXAMPLE 727

### (E or Z)-[2-Cyano-2-(3-ethyl-4-oxo-5-(E/Z)-phenylaminomethylene-thiazolidin-2-ylidene)-N-[3-(2-oxo-pyrrolidin-1-yl)-propyl]-acetamide

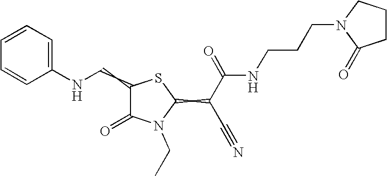

Analogously to Example 721, 36.7 mg of product is obtained from 60 mg of the acid that is described under Example xx) and 29.6 mg of 1-(3-aminopropyl)-2-pyrrolidinone.

¹H-NMR (DMSO-d6, stored with K₂CO₃, main isomer): δ=1.24 (3H), 1.65 (2H), 1.93 (2H), 2.23 (2H), 3.08-3.23 (4H), 3.28-3.38 (2H), 4.22 (2H), 7.05 (1H), 7.22-7.38 (4H), 7.66 (1H), 8.11 (1H), 10.30 (1H) ppm.

### EXAMPLE 728

### (E or Z)-[2-Cyano-N-cyclohexyl-2-(3-ethyl-4-oxo-5-(E/Z)-phenylamino-methylene-thiazolidin-2-ylidene)-acetamide

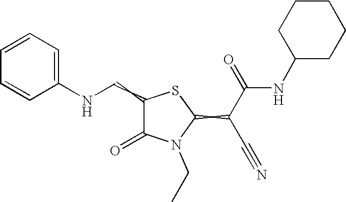

Analogously to Example 721, 24.4 mg of product is obtained from 60 mg of the acid that is described under Example xx) and 21.1 mg of cyclohexylamine.

¹H-NMR (DMSO-d6, stored with K₂CO₃, main isomer): δ=1.24 (3H), 1.25-1.80 (10H), 3.56-3.72 (1H), 4.22 (2H), 6.87 (1H), 7.07 (1H), 7.18-7.40 (4H), 8.08 (1H), 10.27 (1H) ppm.

### EXAMPLE 729

### (E or Z)-[4-[2-Cyano-2-(3-ethyl-4-oxo-5-(E/Z)-phenylaminomethylene-thiazolidin-2-ylidene)-acetylamino]-piperidine-1-carboxylic acid ethyl ester

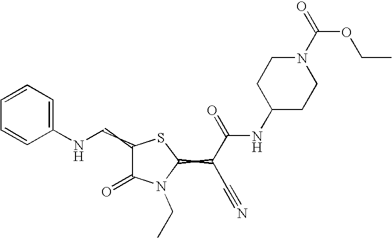

Analogously to Example 721, 41.2 mg of product is obtained from 60 mg of the acid that is described under Example xx) and 36.0 mg of 4-aminopiperidine-1-carboxylic acid ethyl ester.

¹H-NMR (DMSO-d6, stored with K₂CO₃, main isomer): δ=1.19 (3H), 1.24 (3H), 1.50 (2H), 1.65-1.80 (2H), 2.85 (2H), 3.84 (1H), 3.96 (2H), 4.04 (2H), 4.22 (2H), 7.05 (1H), 7.19-7.43 (5H), 8.11 (1H), 10.29 (1H) ppm.

### EXAMPLE 730

### (E or Z)-[2-Cyano-2-(3-ethyl-4-oxo-5-(E/Z)-phenylaminomethylene-thiazolidin-2-ylidene)-N-(3-hydroxy-propyl)-acetamide

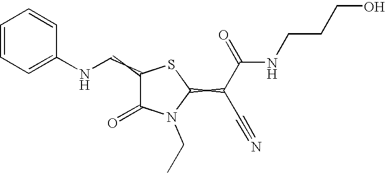

Analogously to Example 721, 61.6 mg of product is obtained from 100 mg of the acid that is described under Example xx) and 26.2 mg of 3-amino-1-propanol.

¹H-NMR (DMSO-d6, stored with K₂CO₃, main isomer): δ=1.23 (3H), 1.63 (2H), 3.36 (2H), 3.46 (2H), 4.23 (2H), 4.53 (1H), 7.05 (1H), 7.20-7.38 (4H), 7.62 (1H), 8.10 (1H), 10.29 (1H) ppm.

### EXAMPLE 731

### (E or Z)-[2-Cyano-2-(3-ethyl-4-oxo-5-(E/Z)-phenylaminomethylene-thiazolidin-2-ylidene)-N-(4-methoxy-benzyl)-acetamide

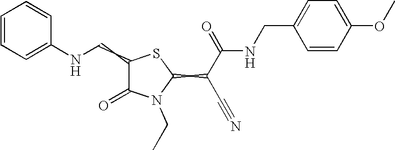

Analogously to Example 721, 35.7 mg of product is obtained from 80.0 mg of the acid that is described under Example xx) and 38.3 mg of 4-methoxybenzylamine.

¹H-NMR (DMSO-d6, stored with K₂CO₃, main isomer): δ=1.23 (3H), 3.73 (3H), 4.22 (2H), 4.27 (2H), 6.88 (2H), 7.04 (1H), 7.20-7.37 (6H), 8.06-8.23 (2H), 10.28 (1H) ppm.

### EXAMPLE 732

### (E or Z)-[2-Cyano-2-(3-ethyl-4-oxo-5-(E/Z)-phenylaminomethylene-thiazolidin-2-ylidene)-N-[2-(4-hydroxy-phenyl)-ethyl]-acetamide

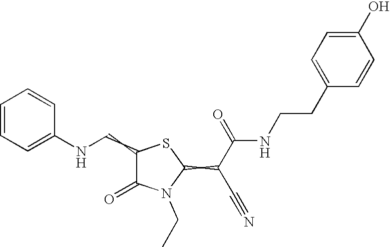

Analogously to Example 721, 19.4 mg of product is obtained from 80.0 mg of the acid that is described under Example xx) and 38.3 mg of 2-(4-hydroxyphenyl)-ethylamine.

¹H-NMR (DMSO-d6, stored with K₂CO₃, main isomer): δ=1.24 (3H), 2.67 (2H), 3.32 (2H), 4.21 (2H), 6.70 (2H), 6.88 (1H), 7.01 (2H), 7.13-7.38 (5H), 8.15 (1H), 9.18 (1H), 10.32 (1H) ppm.

### EXAMPLE 733

### (E or Z)-[N-Allyl-2-cyano-2-(3-ethyl-4-oxo-5-(E/Z)-phenylaminomethylene-thiazolidin-2-ylidene)-acetamide

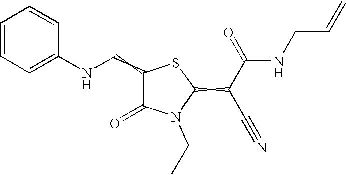

Analogously to Example 721, 65.3 mg of product is obtained from 80.0 mg of the acid that is described under Example xx) and 16.0 mg of allylamine.

¹H-NMR (DMSO-d6, stored with K₂CO₃, main isomer): δ=1.24 (3H), 3.79 (2H), 4.22 (2H), 5.06 (1H), 5.12 (1H), 5.84 (1H), 7.03 (1H), 7.19-7.37 (4H), 7.65-7.76 (1H), 8.12 (1H), 10.29 (1H) ppm.

### EXAMPLE 734

### (E or Z)-[2-Cyano-2-(3-ethyl-4-oxo-5-(E/Z)-phenylaminomethylene-thiazolidin-2-ylidene)-N-(2-hydroxy-ethyl)-acetamide

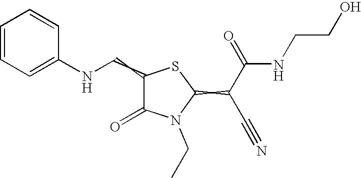

Analogously to Example 721, 15.1 mg of product is obtained from 80.0 mg of the acid that is described under Example xx) and 17.1 mg of ethanolamine.

¹H-NMR (DMSO-d6, stored with K₂CO₃, main isomer): δ=1.22 (3H), 3.25 (2H), 3.46 (2H), 4.21 (2H), 4.73 (1H), 7.00 (1H), 7.10-7.39 (5H), 8.16 (1H), 10.32 (1H) ppm.

### EXAMPLE 735

### (E or Z)-[2-Cyano-2-(3-ethyl-4-oxo-5-(E/Z)-phenylaminomethylene-thiazolidin-2-ylidene)-N-(4-hydroxy-butyl)-acetamide

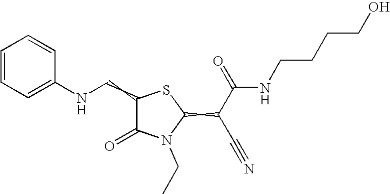

Analogously to Example 721, 57.9 mg of product is obtained from 80.0 mg of the acid that is described under Example xx) and 24.9 mg of 4-amino-1-butanol.

¹H-NMR (DMSO-d6, stored with K₂CO₃, main isomer): δ=1.22 (3H), 1.37-1.56 (4H), 3.17 (2H), 3.40 (2H), 4.21 (2H), 4.39 (1H), 7.01 (1H), 7.12-7.39 (5H), 8.15 (1H), 10.27 (1H) ppm.

### EXAMPLE 736

### (E or Z)-[2-Cyano-2-(3-ethyl-4-oxo-5-(E/Z)-phenylaminomethylene-thiazolidin-2-ylidene)-N-(6-hydroxy-hexyl)-acetamide

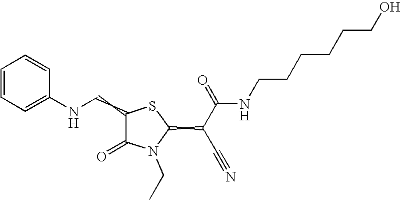

Analogously to Example 721, 10.7 mg of product is obtained from 80.0 mg of the acid that is described under Example xx) and 32.7 mg of 4-amino-1-hexanol.

¹H-NMR (DMSO-d6, stored with K₂CO₃, main isomer): δ=1.16-1.53 (11H), 3.15 (2H), 3.38 (2H), 4.21 (2H), 4.34 (1H), 6.87 (1H), 7.01 (1H), 7.14-7.40 (4H), 8.13 (1H), 10.28 (1H) ppm.

### EXAMPLE 737

### (E or Z)-[2-Cyano-2-(3-ethyl-4-oxo-5-(E/Z)-phenylaminomethylene-thiazolidin-2-ylidene)-acetamide

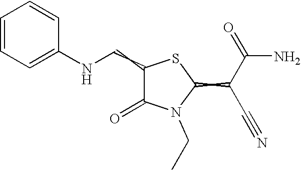

Analogously to Example 721, 73.1 mg of product is obtained from 100 mg of the acid that is described under Example xx) and 0.1 ml of an approximately 7 M solution of ammonia in methanol.

¹H-NMR (DMSO-d6, stored with K₂CO₃, main isomer): δ=1.24 (3H), 4.22 (2H), 7.05 (1H), 7.09-7.40 (6H), 8.10 (1H), 10.34 (1H) ppm.

### EXAMPLE 738

### (E or Z)-[N-Ethyl-2-cyano-2-(3-ethyl-4-oxo-5-(E/Z)-phenylaminomethylene-thiazolidin-2-ylidene)-acetamide

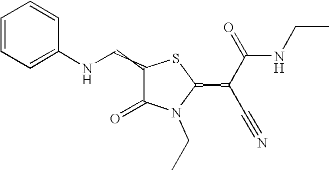

Analogously to Example 721, 144 mg of product is obtained from 200 mg of the acid that is described under Example xx) and 0.35 ml of a 2M solution of ethylamine in THF.

¹H-NMR (DMSO-d6, stored with K₂CO₃, main isomer): δ=1.07 (3H), 1.23 (3H), 3.21 (2H), 4.22 (2H), 7.06 (1H), 7.22-7.40 (4H), 7.66 (1H), 8.10 (1H), 10.28 (1H) ppm.

### EXAMPLE 739

### (E or Z)-[Cyano-(3-ethyl-4-oxo-5-(E/Z)-phenylaminomethylene-thiazolidin-2-ylidene)-acetic acid-3-hydroxy-propyl ester

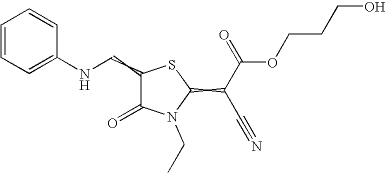

100 mg of the acid that is produced in Reference Example x is dissolved in 1.25 ml of dimethylformamide, mixed with 112 mg of TBTU, 34.5 μl of triethylamine, 10 mg of 4-N,N-dimethylaminopyridine and 50.6 μl of 1,3-propanediol, and it is stirred for 4 hours between 60 and 90° C. and for 16 hours at 25° C. It is diluted with 70 ml of ethyl acetate, and it is washed once with 10 ml of saturated sodium bicarbonate solution, once with 10 ml of 1N sulfuric acid and once with 10 ml of water. After drying on sodium sulfate and filtration, it is concentrated by evaporation in a vacuum. The crude product that is obtained is purified by column chromatography on silica gel and hexane/0-100% ethyl acetate/0-20% ethanol. 29.8 mg of the desired product is obtained in this way.

¹H-NMR (DMSO-d6, stored with K₂CO₃, main isomer): δ=1.25 (3H), 1.79 (2H), 3.52 (2H), 4.19-4.31 (4H), 4.57 (1H), 7.10 (1H), 7.29-7.41 (4H), 8.21 (1H), 10.55 (1H) ppm.

### EXAMPLE 740

### (E or Z)-[Cyano-(3-ethyl-4-oxo-5-(E/Z)-phenylaminomethylene-thiazolidin-2-ylidene)-acetic acid-2-(2-hydroxy-ethoxy)-ethyl ester

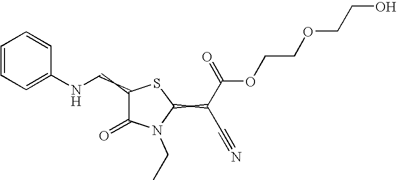

Analogously to Example 739, 59.6 mg of product is obtained from 100 mg of the acid that is described under Example xx) and 66.0 μl of diethylene glycol.

¹H-NMR (DMSO-d6, stored with K₂CO₃, main isomer): δ=1.25 (3H), 3.46-3.54 (4H), 3.69 (2H), 4.20-4.35 (4H), 4.62 (1H), 7.10 (1H), 7.29-7.41 (4H), 8.22 (1H), 10.55 (1H) ppm.

### EXAMPLE 741

### (E or Z)-[Cyano-(3-ethyl-4-oxo-5-(E/Z)-phenylaminomethylene-thiazolidin-2-ylidene)-acetic acid-2-[bis-(2-hydroxy-ethyl)-amino]-ethyl ester

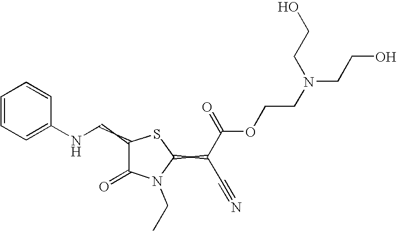

Analogously to Example 739, 17.9 mg of product is obtained from 100 mg of the acid that is described under Example xx) and 139 μl of triethanolamine.

¹H-NMR (DMSO-d6, stored with K₂CO₃, main isomer): δ=1.25 (3H), 2.63 (4H), 2.83 (2H), 3.44 (4H), 4.17-4.41 (6H), 7.06-7.15 (1H), 7.25-7.42 (4H), 8.17-8.26 (1H), 10.48-10.62 (1H) ppm.

### EXAMPLE 742

### (E or Z)-[Cyano-(3-ethyl-4-oxo-5-(E/Z)-phenylaminomethylene-thiazolidin-2-ylidene)-acetic acid-4-hydroxymethyl-phenyl ester

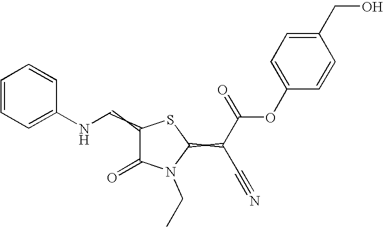

Analogously to Example 739, 47.1 mg of product is obtained from 100 mg of the acid that is described under Example xx) and 86.9 mg of 4-hydroxy benzyl alcohol.

¹H-NMR (DMSO-d6, stored with K₂CO₃, main isomer): δ=1.30 (3H), 4.32 (2H), 4.52 (2H), 5.25 (1H), 7.09 (1H), 7.16 (2H), 7.23-7.44 (6H), 8.27 (1H), 10.66 (1H) ppm.

### EXAMPLE 743

### (E or Z)-[Cyano-(3-ethyl-4-oxo-5-(E/Z)-phenylaminomethylene-thiazolidin-2-ylidene)-acetic acid-4-(3-hydroxy-propyl)-phenyl ester

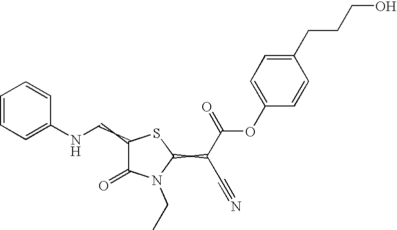

Analogously to Example 739, 51.3 mg of product is obtained from 100 mg of the acid that is described under Example xx) and 106.5 mg of 3-(4-hydroxyphenyl)propanol.

¹H-NMR (DMSO-d6, stored with K₂CO₃, main isomer): δ=1.31 (3H), 1.73 (2H), 2.64 (2H), 3.43 (2H), 4.32 (2H), 4.49 (1H), 7.07-7.16 (3H), 7.26 (2H), 7.30-7.43 (4H), 8.21-8.30 (1H), 10.60-10.70 (1H) ppm.

### EXAMPLE 744

### (E or Z)-[Cyano-(3-ethyl-4-oxo-5-(E/Z)-phenylaminomethylene-thiazolidin-2-ylidene)-acetic acid-3-(2-hydroxy-ethyl)-phenyl ester

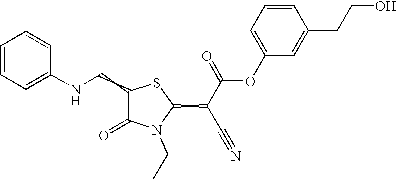

Analogously to Example 739, 32.8 mg of product is obtained from 100 mg of the acid that is described under Example xx) and 89.3 μl of 2-(3-hydroxyphenyl)ethanol.

¹H-NMR (DMSO-d6, stored with K₂CO₃, main isomer): δ=1.31 (3H), 2.76 (2H), 3.63 (2H), 4.32 (2H), 4.67 (1H), 7.01-7.18 (4H), 7.23-7.43 (5H), 8.22-8.31 (1H), 10.61-10.69 (1H) ppm.

### EXAMPLE 745

### (E or Z)-[Cyano-(3-ethyl-4-oxo-5-(E/Z)-phenylaminomethylene-thiazolidin-2-ylidene)-acetic acid-4,4,4-trifluorobutyl ester

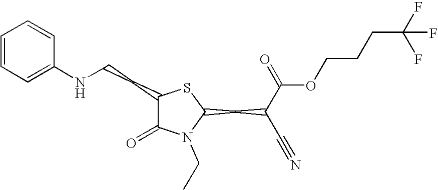

Analogously to Example 739, 28.0 mg of product is obtained from 100 mg of the acid that is described under Example xx) and 34.5 μl of 4,4,4,-trifluorobutanol.

¹H-NMR (DMSO-d6, stored with K₂CO₃, main isomer): δ=1.25 (3H), 1.90 (2H), 2.38 (2H), 4.18-4.33 (4H), 7.11 (1H), 7.28-7.44 (5H), 8.21 (1H), 10.56 (1H) ppm.

### EXAMPLE 746

### (E or Z)-Cyano-(3-ethyl-4-oxo-5-(E/Z)-phenylaminomethylene-thiazolidin-2-ylidene)-acetic acid-4-hydroxymethyl benzyl ester

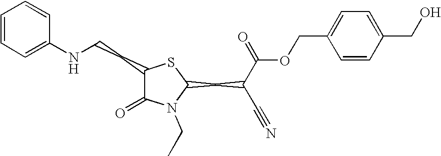

Analogously to Example 739, 39.4 mg of product is obtained from 100 mg of the acid that is described under Example xx) and 96.7 mg of 1,4-benzenedimethanol.

¹H-NMR (DMSO-d6, stored with K₂CO₃, main isomer): δ=1.24 (3H), 4.25 (2H), 4.49 (2H), 5.20 (1H), 5.25 (2H), 7.11 (1H), 7.26-7.44 (8H), 8.21 (1H), 10.55 (1H) ppm.

### EXAMPLE 747

### (E or Z)-Cyano-(3-ethyl-4-oxo-5-(E/Z)-phenylaminomethylene-thiazolidin-2-ylidene)-acetic acid-2-(2-hydroxy-ethyl)-phenyl ester

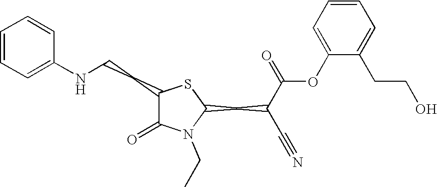

Analogously to Example 739, 32.0 mg of product is obtained from 100 mg of the acid that is described under Example xx) and 83.7 μl of 2-(hydroxyphenyl)-ethanol.

¹H-NMR (DMSO-d6, stored with K₂CO₃, main isomer): δ=1.32 (3H), 2.69 (2H), 3.61 (2H), 4.32 (2H), 4.68 (1H), 7.02-7.44 (9H), 8.26 (1H), 10.65 (1H) ppm.

### EXAMPLE 748

### (E or Z)-[Cyano-(3-ethyl-4-oxo-5-(E/Z)-phenylaminomethylene-thiazolidin-2-ylidene)-acetic acid-2-(4-bromo-phenyl)-2-oxo-ethyl ester

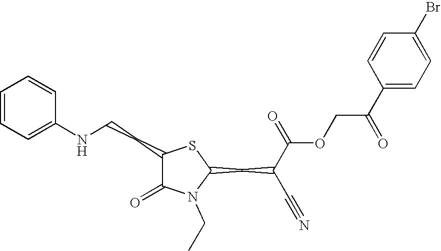

300 mg of the acid that is produced in Reference Example x is dissolved in a mixture that consists of 3 ml of acetone and 0.9 ml of DMSO and mixed with 73.8 mg of lithium carbonate and 277.6 mg of 2,4′-dibromoacetophenone. After 18 hours of stirring at 25° C., it is diluted with 200 ml of ethyl acetate and washed twice with 20 ml each of semi-concentrated sodium chloride solution. After drying on sodium sulfate and filtration, it is concentrated by evaporation in a vacuum. The crude product that is obtained is purified by column chromatography on silica gel and hexane/0-40% ethyl acetate. In this way, 278.4 mg of the desired product is obtained.

¹H-NMR (DMSO-d6, stored with K₂CO₃, main isomer): δ=1.25 (3H), 4.26 (2H), 5.59 (2H), 7.08 (1H), 7.13-7.48 (4H), 7.63-8.05 (4H), 8.24 (1H), 10.56 (1H) ppm.

Production of the Intermediate Compounds that Preferably can be Used for the Production of the Thiazolidinones According to the Invention:

### EXAMPLE a)

### Cyano-ethylthiocarbamoyl-acetic acid ethyl ester

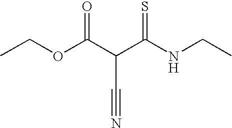

4.25 ml of ethyl isothiocyanate is added to a mixture that consists of 5 g of cyanoacetic acid ethyl ester and 5 ml of triethylamine at 25° C. Then, it is allowed to stir for 6 more hours at 50° C. Then, the reaction mixture is concentrated by evaporation in a vacuum. The residue is taken up in ethanol and poured onto 150 ml of ice-cold 1N hydrochloric acid. It is allowed to stir for 3 more hours at 25° C., and then the residue is filtered off. The solid that is obtained is rewashed with water. 7 g of product is obtained.

### EXAMPLE b)

### (E or Z)-Cyano-(3-ethyl-4-oxo-thiazolidin-2-ylidene)-acetic acid ethyl ester

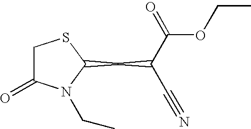

7.82 g of the compound that is described under Example a) is dissolved in 100 ml of tetrahydrofuran. A solution of 3.9 ml of bromoacetyl chloride is slowly added and allowed to stir for 8 hours at 25° C. Then, the reaction mixture is poured onto saturated aqueous sodium bicarbonate solution. It is allowed to stir for 1 more hour and then extracted with ethyl acetate. The organic phase is washed with saturated sodium chloride solution, dried on sodium sulfate and concentrated by evaporation in a vacuum. The crude product that is obtained is recrystallized from a mixture of ethyl acetate/diisopropyl ester. 7.7 g of product is obtained.

¹H-NMR (CDCl₃): δ=1.36 (6H); 3.70 (2H); 4.32 (4H) ppm.

### EXAMPLE c)

### (E or Z)-Cyano-(5-(E/Z)-ethoxymethylene-3-ethyl-4-oxo-thiazolidin-2-ylidene)-acetic acid ethyl ester

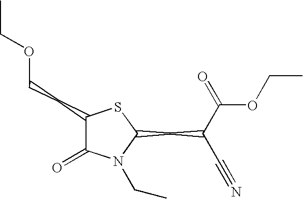

A mixture that consists of 1.54 g of the substance that is described under Example b), 2.5 ml of triethyl orthoformate and 3.5 ml of acetic acid anhydride is refluxed for 8 hours. Then, the reaction mixture is poured onto ice water. It is allowed to stir for 3 more hours, and then the residue is filtered off. The solid that is obtained is rewashed with water. 1.28 g of product is obtained.

¹H-NMR (CDCl₃): δ=1.38 (9H); 4.20-4.40 (6H); 7.72 (1H) ppm.

### EXAMPLE d)

### 2-Ethylthiocarbamoyl-malonic acid diethyl ester

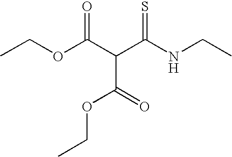

Analogously to Example a), 8.5 g of product is obtained from 6 g of malonic acid diethyl ester, 5.7 ml of triethylamine and 4.9 ml of ethyl isothiocyanate.

### EXAMPLE e)

### 2-(3-Ethyl-4-oxo-thiazolidin-2-ylidene)-malonic acid diethyl ester

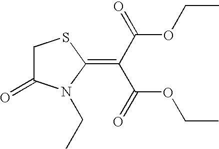

Analogously to Example b), 10.2 g of product is obtained from 12.5 g of the substance that is described under Example d) and 5 ml of bromoacetyl chloride in tetrahydrofuran.

¹H-NMR (CDCl₃): δ=1.16 (3H); 1.25 (3H); 1.31 (3H); 3.66 (2H); 3.76 (2H); 4.20-4.35 (4H) ppm.

### EXAMPLE f)

### 2-(5-(E/Z)-Ethoxymethylene-3-ethyl-4-oxo-thiazolidin-2-ylidene)-malonic acid diethyl ester

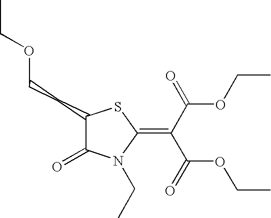

Analogously to Example c), 1.3 g of product is obtained from 1.8 g of the compound that is described under Example e), 2.5 ml of triethyl orthoformate and 3.5 ml of acetic acid anhydride.

¹H-NMR (CDCl₃): δ=1.15-1.40 (12H); 3.75 (2H); 4.20-4.45 (6H); 7.75 (1H) ppm.

### EXAMPLE g)

### 2,2-Dicyano-N-ethyl-thioacetamide

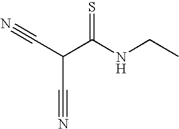

Analogously to Example a), 31.8 g of product is obtained from 20 g of malonic acid dinitrile, 20 ml of triethylamine and 17 ml of ethyl isothiocyanate.

### EXAMPLE h)

### 2-(3-Ethyl-4-oxo-thiazolidin-2-ylidene)-malononitrile

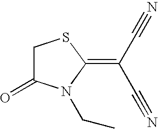

Analogously to Example b), 8.1 g of product is obtained from 8.73 g of the substance that is described under Example g) and 4.8 ml of bromoacetyl chloride in tetrahydrofuran.

¹H-NMR (CDCl₃): δ=1.36 (3H); 4.00 (2H); 4.19 (2H) ppm.

### EXAMPLE i)

### 2-(5-(E/Z)-Ethoxymethylene-3-ethyl-4-oxo-thiazolidin-2-ylidene)-malononitrile

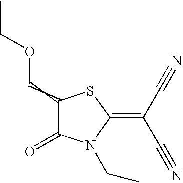

Analogously to Example c), 3.4 g of product is obtained from 3.4 g of the compound that is described under Example h), 6.9 ml of triethyl orthoformate and 9.6 ml of acetic acid anhydride.

¹H-NMR (CDCl₃): δ=1.31 (3H); 1.39 (3H); 4.18-4.35 (4H); 7.81 (1H) ppm.

### EXAMPLE j)

Cyano-ethylthiocarbamoyl-acetic acid propyl ester

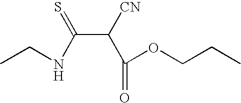

Analogously to Example a), 5.6 g of product is obtained from 3.5 g of cyanoacetic acid propyl ester, 3.5 ml of triethylamine and 2.55 ml of ethyl isothiocyanate.

### EXAMPLE k)

### (E or Z)-Cyano-(3-ethyl-4-oxo-thiazolidin-2-ylidene)-acetic acid propyl ester

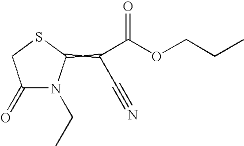

Analogously to Example b), 4.95 g of product is obtained from 7 g of the compound that is described under 1) and 2.7 ml of bromoacetyl chloride in 100 ml of tetrahydrofuran.

¹H-NMR (CDCl₃): δ=1.00 (3H); 1.37 (3H); 1.73 (2H); 3.69 (2H); 4.20 (2H); 4.31 (2H) ppm.

### EXAMPLE 1)

### (E or Z)-Cyano-(5-(E/Z)-ethoxymethylene-3-ethyl-4-oxo-thiazolidin-2-ylidene)-acetic acid propyl ester

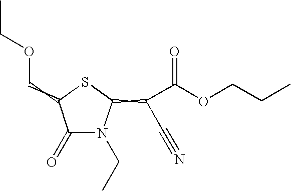

Analogously to Example c), 4.26 g of product is obtained from 4.95 g of the compound that is described under 2), 7.45 ml of triethyl orthoformate and 10 ml of acetic acid anhydride.

¹H-NMR (CDCl₃): δ=0.99 (3H); 1.30-1.45 (6H); 1.75 (2H); 4.15-4.30 (4H); 4.38 (2H); 7.71 (1H) ppm.

### EXAMPLE m)

### Cyano-ethylthiocarbamoyl-acetic acid isopropyl ester

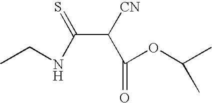

Analogously to Example a), 6.7 g of product is obtained from 4 g of cyanoacetic acid isopropyl ester, 4 ml of triethylamine and 3 ml of ethyl isothiocyanate.

### EXAMPLE n)

### (E or Z)-Cyano-(3-ethyl-4-oxo-thiazolidin-2-ylidene)-acetic acid isopropyl ester

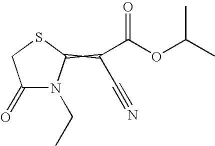

Analogously to Example b), 6.18 g of product is obtained from 6.7 g of the compound that is described under 1) and 3.15 ml of bromoacetyl chloride in 100 ml of tetrahydrofuran.

1H-NMR (CDCl₃): δ=1.28-1.40 (9H); 3.70 (2H); 4.30 (2H); 5.13 (1H) ppm.

### EXAMPLE o)

### (E or Z)-Cyano-(5-(E/Z)-ethoxymethylene-3-ethyl-4-oxo-thiazolidin-2-ylidene)-acetic acid isopropyl ester

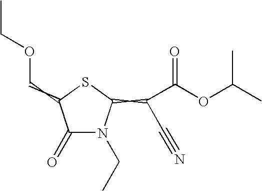

Analogously to Example c), 1.77 g of product is obtained from 2 g of the compound that is described under 2), 3 ml of triethyl orthoformate and 4.3 ml of acetic acid anhydride.

1H-NMR (CDCl₃): δ=1.25-1.45 (12H); 4.23 (2H); 4.37 (2H); 5.12 (1H); 7.70 (1H) ppm.

### EXAMPLE p)

### Cyano-ethylthiocarbamoyl-acetic acid-tert-butyl ester

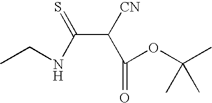

Analogously to Example a), 8 g of product is obtained from 5 g of cyanoacetic acid tert-butyl ester, 5.6 ml of triethylamine and 5 ml of ethyl isothiocyanate.

### EXAMPLE q)

### (E or Z)-Cyano-(3-ethyl-4-oxo-thiazolidin-2-ylidene)-acetic acid-tert-butyl ester

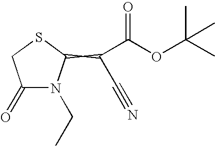

Analogously to Example b), 7.1 g of product is obtained from 9.8 g of the compound that is described under 1) and 3.6 ml of bromoacetyl chloride in 150 ml of tetrahydrofuran.

¹H-NMR (CDCl₃): δ=1.32 (3H); 1.55 (9H); 3.68 (2H); 4.30 (2H) ppm.

### EXAMPLE r)

### (E or Z)-Cyano-(5-(E/Z)-ethoxymethylene-3-ethyl-4-oxo-thiazolidin-2-ylidene)-acetic acid-tert-butylester

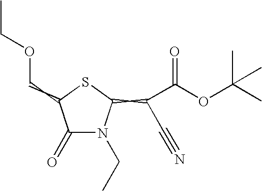

Analogously to Example c), 4.6 g of product is obtained from 6.16 g of the compound that is described under 2), 8.8 ml of triethyl orthoformate and 12.6 ml of acetic acid anhydride.

¹H-NMR (CDCl₃): δ=1.30-1.45 (6H); 1.55 (9H); 4.24 (2H); 4.35 (2H); 7.69 (1H) ppm.

### EXAMPLE s)

### (E or Z)-Cyano-(3-ethyl-4-oxo-thiazolidin-2-ylidene)-acetic acid benzyl ester

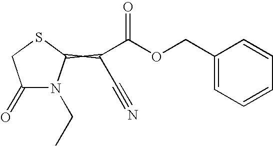

A solution of 1.75 g of cyanoacetic acid benzyl ester in 10 ml of dimethylformamide is added to a suspension of 0.4 g of sodium hydride (60%) in 5 ml of dimethylformamide at 0° C. It is stirred for 10 more minutes at 0° C., and then a solution of 876 μl of ethyl isothiocyanate in 5 ml of dimethylformamide is added. Then, it is stirred for 2 more hours at 25° C. Then, at 0° C., a solution of 1 ml of bromoacetyl chloride in 5 ml of dimethylformamide is added, and it is stirred for 15 more hours at 25° C. Then, the reaction mixture is poured onto saturated sodium bicarbonate solution. It is extracted with dichloromethane, the organic phase is washed with saturated sodium chloride solution, dried on sodium sulfate and concentrated by evaporation in a vacuum. The crude product is purified by column chromatography on silica gel with a mixture that consists of hexane/ethyl acetate. 1.1 g of product is obtained.

1H-NMR (CDCl₃): δ=1.35 (3H); 3.70 (2H); 4.30 (2H); 5.31 (2H), 7.30-7.48 (5H) ppm.

### EXAMPLE t)

### (E or Z)-Cyano-(5-(E/Z)-ethoxymethylene-3-ethyl-4-oxo-thiazolidin-2-ylidene)-acetic acid benzyl ester

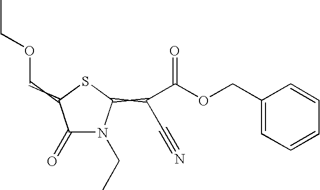

Analogously to Example c), 1.26 g of product is obtained from 11 g of the compound that is described under 1), 1.49 ml of triethyl orthoformate and 2.1 ml of acetic acid anhydride.

¹H-NMR (CDCl₃): δ=1.30-1.45 (6H); 4.25 (2H); 4.38 (2H); 5.29 (2H); 7.30-7.48 (5H), 7.72 (1H) ppm.

### EXAMPLE u)

### 2-Cyano-2-ethylthiocarbamoyl-N,N-dimethyl-acetamide

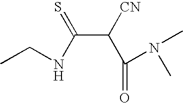

Analogously to Example a), 3.3 g of product is obtained from 3 g of N,N-dimethyl cyanoacetamide, 4 ml of triethylamine and 2.8 ml of ethyl isothiocyanate.

### EXAMPLE v)

### 2-(E or Z)-Cyano-2-(3-ethyl-4-oxo-thiazolidin-2-ylidene)-N,N-dimethyl-acetamide

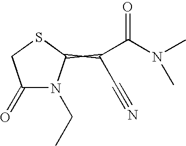

Analogously to Example b), 1.77 g of product is obtained from 2.3 g of the compound that is described under 1) and 1.54 ml of bromoacetyl chloride in 70 ml of tetrahydrofuran.

¹H-NMR (CDCl₃): δ=1.33 (3H); 3.05-3.20 (6H); 3.70 (2H); 4.24 (2H) ppm.

### EXAMPLE w)

### 2-(E or Z)-Cyano-2-(5-(E/Z)-ethoxymethylene-3-ethyl-4-oxo-thiazolidin-2-ylidene)-N,N-dimethyl-acetamide

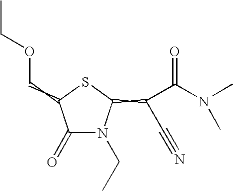

Analogously to Example c), 1.65 g of product is obtained from 1.77 g of the compound that is described under 2), 2.83 ml of triethyl orthoformate and 4.05 ml of acetic acid anhydride.

¹H-NMR (CDCl₃): δ=1.30-1.40 (6H); 3.05-3.15 (611); 4.20 (2H); 4.31 (2H); 7.63 (1H) ppm.

### EXAMPLE x)

### 2-Cyano-N-ethyl-3-oxo-3-phenyl-thiopropionamide

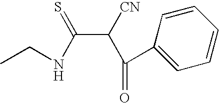

Analogously to Example a), 2.24 g of product is obtained from 1.5 g of benzoyl acetonitrile, 1.6 ml of triethylamine and 1.45 ml of ethyl isothiocyanate.

### EXAMPLE y)

### 2-(E or Z)-(3-Ethyl-4-oxo-thiazolidin-2-ylidene)-3-oxo-3-phenyl-propionitrile

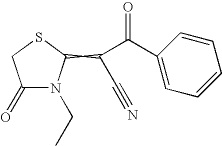

Analogously to Example b), 1.82 g of product is obtained from 2.24 g of the compound that is described under 1) and 1.29 ml of bromoacetyl chloride in 50 ml of tetrahydrofuran.

¹H-NMR (CDCl₃): δ=1.43 (3H); 3.71 (2H); 4.43 (2H); 7.48-7.60 (3H); 7.80-7.88 (2H) ppm.

### EXAMPLE z)

### 2-(E or Z)-(5-(E/Z)-Ethoxymethylene-3-ethyl-4-oxo-thiazolidin-2-ylidene)-3-oxo-3-phenyl-propionitrile

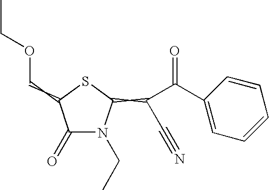

Analogously to Example c), 1.46 g of product is obtained from 1.8 g of the compound that is described under 2), 2.52 ml of triethyl orthoformate and 3.63 ml of acetic acid anhydride.

¹H-NMR (CDCl₃): δ=1.38-1.50 (6H); 4.31 (2H); 4.49 (2H); 7.40-7.58 (3H); 7.80-7.88 (3H) ppm.

### EXAMPLE aa)

### 3-Ethyl-2-(E or Z)-(2-oxo-1,2-diphenyl-ethylidene)-thiazolidin-4-one

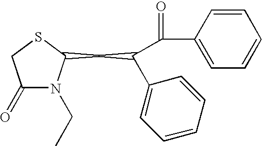

A solution of 1.96 g of benzyl phenyl ketone in 10 ml of dimethylformamide is added to a suspension of 0.4 g of sodium hydride (60%) in 5 ml of dimethylformamide at 0° C. It is stirred for 10 more minutes at 0° C., and then a solution of 876 μl of ethyl isothiocyanate in 5 ml of dimethylformamide is added. Then, it is stirred for 2 more hours at 25° C. Then, a solution of 1 ml of bromoacetyl chloride in 5 ml of dimethylformamide is added at 0° C., and it is stirred for 15 more hours at 25° C. Then, the reaction mixture is poured onto saturated sodium bicarbonate solution. It is extracted with dichloromethane, the organic phase is washed with saturated sodium chloride solution, dried on sodium sulfate and concentrated by evaporation in a vacuum. The crude product is purified by column chromatography on silica gel with a mixture that consists of hexane/ethyl acetate. 1.24 g of product is obtained.

1H-NMR (CDCl₃): δ=0.74 (3H); 3.25 (2H); 3.70 (2H); 7.10-7.30 (10H) ppm.

### EXAMPLE ab)

### (E or Z)-(3-Ethyl-4-oxo-thiazolidin-2-ylidene)-phenyl-acetonitrile

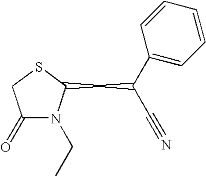

A solution of 1.15 g of benzyl cyanide in 10 ml of dimethylformamide is added to a suspension of 0.4 g of sodium hydride (60%) in 5 ml of dimethylformamide at 0° C. It is stirred for 10 more minutes at 0° C., and then a solution of 87611 of ethyl isothiocyanate in 5 ml of dimethylformamide is added. Then, it is stirred for 2 more hours at 25° C. Then, a solution of 1 ml of bromoacetyl chloride in 5 ml of dimethylformamide is added at 0° C., and it is stirred for 15 more hours at 25° C. Then, the reaction mixture is poured onto saturated sodium bicarbonate solution. It is extracted with dichloromethane, the organic phase is washed with saturated sodium chloride solution, dried on sodium sulfate and concentrated by evaporation in a vacuum. The crude product is purified by column chromatography on silica gel with a mixture that consists of hexane/ethyl acetate. 1.4 g of product is obtained.

¹H-NMR (CDCl₃): δ=1.45 (3H); 3.71 (2H); 4.30 (2H); 7.30-7.50 (5H) ppm.

### EXAMPLE ac)

### 2-(tert-Butyl-diphenyl-silanyloxy)-ethylamine

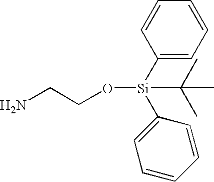

34 g of imidazole and 78 ml of tert.butyl diphenyl silyl chloride are added to a solution of 15 ml of 2-aminoethanol in 150 ml of N,N-dimethylformamide at 0° C. It is allowed to stir for 16 more hours at 25° C. Then, the reaction mixture is poured onto ice-cold saturated sodium bicarbonate solution. It is extracted with ethyl acetate, the organic phase is washed with saturated sodium chloride solution, dried on sodium sulfate and concentrated by evaporation in a vacuum. The crude product is purified by column chromatography on silica gel with a mixture that consists of hexane/ethyl acetate. 45.4 g of product is obtained.

### EXAMPLE ad)

### tert-Butyl-(2-isothiocyano-ethoxy)-diphenylsilane

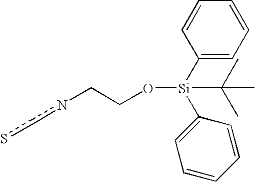

A solution of 5.23 ml of thiophosgene in 50 ml of tetrahydrofuran is slowly added to a solution of 18.7 g of the compound, described under 1), in 250 ml of tetrahydrofuran, at 0° C. Then, it is allowed to stir for 1.5 more hours at 25° C. Then, the reaction mixture is poured onto ice water. It is extracted with ethyl acetate, the organic phase is washed with saturated sodium chloride solution, dried on sodium sulfate and concentrated by evaporation in a vacuum. The crude product is purified by column chromatography on silica gel with a mixture that consists of hexane/ethyl acetate. 7.9 g of product is obtained.

1H-NMR (CDCl₃): δ=1.08 (9H); 3.58 (2H); 3.79 (2H); 7.38-7.48 (6H); 7.65-7.70 (4H) ppm.

### EXAMPLE ae)

### [2-(tert-Butyl-diphenyl-silanyloxy)-ethylthiocarbamoyl]-cyano-acetic acid ethyl ester

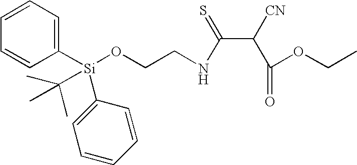

8.9 g of the compound, produced under 2), in 2 ml of tetrahydrofuran is added to a solution of 2.53 ml of cyanoacetic acid ethyl ester and 3.5 ml of triethylamine. It is stirred for 16 more hours at 75° C. Then, it is concentrated by evaporation in a vacuum. The residue is taken up in ethanol and poured onto ice-cold 2N hydrochloric acid. It is allowed to stir for one more hour at 25° C. and then extracted with dichloromethane. The organic phase is washed with saturated sodium chloride solution, dried on sodium sulfate and concentrated by evaporation in a vacuum. 10.7 g of product is obtained.

### EXAMPLE af)

### (E or Z)-{3-[2-(tert-Butyl-diphenyl-silanyloxy)-ethyl]-4-oxo-thiazolidin-2-ylidene}-cyanoacetic acid ethyl ester

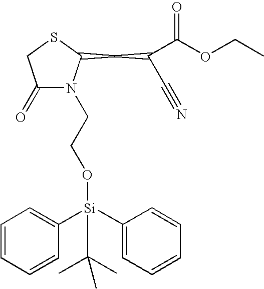

A solution of 2.2 ml of bromoacetyl chloride in 20 ml of tetrahydrofuran is slowly added to a solution of 10.7 g of the compound, described under 3), in 250 ml of tetrahydrofuran. It is allowed to stir for 5 more hours at 25° C., and then the reaction mixture is poured into saturated sodium bicarbonate solution. It is allowed to stir for one more hour and then extracted with ethyl acetate. The organic phase is washed with saturated sodium chloride solution, dried on sodium sulfate and concentrated by evaporation in a vacuum. The crude product is purified by column chromatography on silica gel with a mixture that consists of hexane/ethyl acetate. 6.87 g of product is obtained.

1H-NMR (CDCl₃): δ=0.97-1.05 (9H); 1.34 (3H); 3.59 (2H); 3.95 (2H); 4.29 (2H); 4.58 (2H); 7.30-7.48 (6H); 7.55-7.65 (4H) ppm.

### EXAMPLE ag)

### (E or Z)-{3-[2-(tert-Butyl-diphenyl-silanyloxy)-ethyl]-5-(E/Z)-ethoxymethylene-4-oxo-thiazolidin-2-ylidene}-cyanoacetic acid ethyl ester

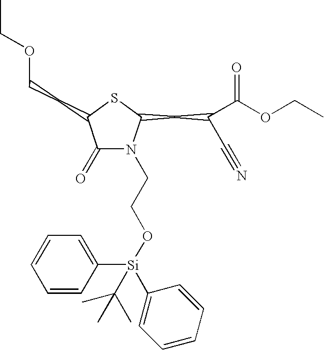

Analogously to Example c), 2.0 g of product is obtained from 2 g of the compound that is described under 4), 1.57 ml of triethyl orthoformate and 2.2 ml of acetic acid anhydride.

1H-NMR (CDCl₃): δ=0.95-1.00 (9H); 1.30-1.48 (6H); 3.93 (2H); 4.22-4.35 (4H); 4.62 (2H); 7.30-7.45 (6H); 7.55-7.62 (4H); 7.68 (1H) ppm.

### EXAMPLE ah)

### Cyano-(2-methoxy-ethylthiocarbamoyl)-acetic acid ethyl ester

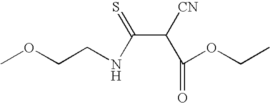

Analogously to Example a), 1.49 g of product is obtained from 1 g of cyanoacetic acid ethyl ester, 1 ml of triethylamine and 1.14 g of 2-methoxy ethyl isothiocyanate.

### EXAMPLE ai)

### (E or Z)-Cyano-[3-(2-methoxy-ethyl)-4-oxo-thiazolidin-2-ylidene]-acetic acid ethyl ester

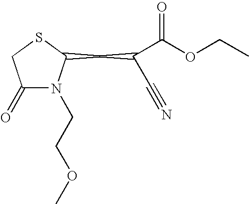

Analogously to Example b), 940 mg of product is obtained from 1.49 g of the compound that is described under 1) and 645 μl of bromoacetyl chloride in 7 ml of tetrahydrofuran.

1H-NMR (CDCl₃): δ=1.35 (3H); 3.35 (3H); 3.69 (2H); 3.74 (2H); 4.30 (2H); 4.56 (2H) ppm.

### EXAMPLE aj)

### (E or Z)-Cyano-[5-(E/Z)-ethoxymethylene-3-(2-methoxy-ethyl)-4-oxo-thiazolidin-2-ylidene]-acetic acid ethyl ester

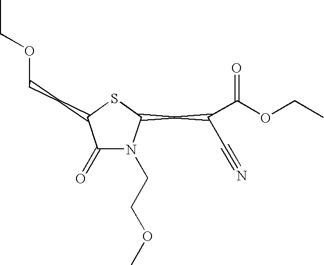

Analogously to Example c), 675 mg of product is obtained from 940 mg of the compound that is described under 2), 1.3 ml of triethyl orthoformate and 1.8 ml of acetic acid anhydride.

1H-NMR (CDCl₃): δ=1.32-1.42 (6H); 3.33 (3H); 3.70 (2H); 4.20-4.35 (4H); 4.59 (2H), 7.72 (1H) ppm.

### EXAMPLE ak)

### Cyano-methylthiocarbamoyl-acetic acid ethyl ester

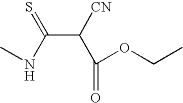

Analogously to Example a), 6 g of product is obtained from 5 g of cyanoacetic acid propyl ester, 5 ml of triethylamine and 3.6 g of methyl isothiocyanate.

### EXAMPLE al)

### (E or Z)-Cyano-(3-methyl-4-oxo-thiazolidin-2-ylidene)-acetic acid ethyl ester

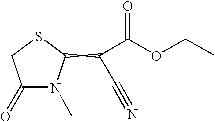

Analogously to Example b), 4.35 g of product is obtained from 4.95 g of the compound that is described under 1) and 2.7 ml of bromoacetyl chloride in 100 ml of tetrahydrofuran.

1H-NMR (CDCl₃): δ=1.35 (3H); 3.70 (3H); 3.73 (2H); 4.32 (2H) ppm.

### EXAMPLE am)

### (E or Z)-Cyano-(5-(E/Z)-ethoxymethylene-3-methyl-4-oxo-thiazolidin-2-ylidene)-acetic acid ethyl ester

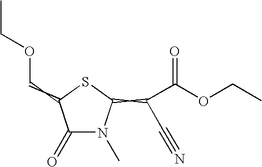

Analogously to Example c), 3.5 g of product is obtained from 4.33 g of the compound that is described under 2), 7.4 ml of triethyl orthoformate and 10 ml of acetic acid anhydride.

1H-NMR (CDCl₃): δ=1.32-1.42 (6H); 3.72 (3H); 4.20-4.38 (2H); 7.71 (1H) ppm.

### EXAMPLE an)

### Isothiocyanato-cyclobutane

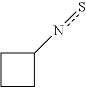

2.0 g of cyclobutylamine is introduced into 50 ml of THF, mixed at 0° C. with 2.3 ml of thiophosgene and stirred for 30 minutes at room temperature. The reaction mixture is mixed with sodium bicarbonate solution and extracted with ethyl acetate. After the solvent is removed, 3 g of the title compound is obtained as a crude product.

1H-NMR (CDCl₃): δ=1.63-1.93 (2H); 2.15-2.50 (4H); 4.05 (1H) ppm.

### EXAMPLE ao)

### Cyano-cyclobutylthiocarbamoyl-acetic acid ethyl ester

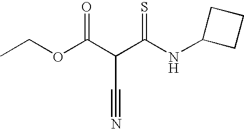

Analogously to Example a), 2.6 g of the title compound is obtained from 2.7 g of cyanoacetic acid ethyl ester, 4.3 ml of triethylamine and 3.0 g of the compound that is described under Example an) after purification by chromatography on silica gel (dichloromethane/methanol 80:20).

### EXAMPLE ap)

### (E or Z)-Cyano-(3-cyclobutyl-4-oxo-thiazolidin-2-ylidene)-acetic acid ethyl ester

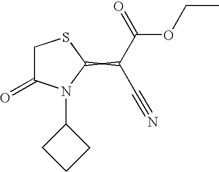

Analogously to Example b), 340 mg of the title compound is obtained from 2.0 g of the compound that is described under Example ao) and 1.1 ml of bromoacetyl chloride in tetrahydrofuran after recrystallization from ethanol.

¹H-NMR (CDCl₃): δ=1.35 (3H); 1.70-1.95 (2H); 2.40-2.52 (2H); 2.70-2.90 (2H); 3.65 (2H); 4.30 (2H); 5.10 (1H) ppm.

### EXAMPLE aq)

### (E or Z)-Cyano-(3-cyclobutyl-5-(E or Z)-ethoxymethylene-4-oxo-thiazolidin-2-ylidene)-acetic acid ethyl ester

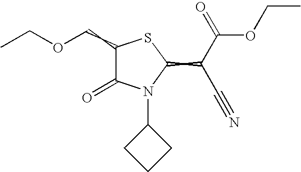

Analogously to Example c), 434 g of the title compound is obtained from 450 mg of the compound that is described under Example ap), 0.66 ml of triethyl orthoformate and 0.93 ml of acetic acid anhydride after recrystallization.

¹H-NMR (CDCl₃): δ=1.30-1.45 (6H); 1.70-1.98 (2H); 2.35-2.52 (2H); 2.80-3.00 (2H); 4.15-4.38 (4H); 5.20 (1H); 7.65 (1H) ppm.

### EXAMPLE ar)

### (E or Z)-{5-(E/Z)-[(3-Bromomethyl-phenylamino)-methylene]-3-ethyl-4-oxo-thiazolidin-2-ylidene}-cyanoacetic acid ethyl ester

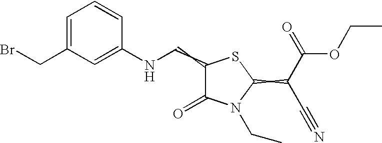

752 mg of the compound that is described under Example 60), 2.70 g of triphenylphosphine and 2.66 g of carbon tetrabromide are dissolved in 100 ml of THF and stirred for 1 hour at room temperature. The reaction mixture is mixed with water and extracted with ethyl acetate. After purification by chromatography on silica gel, 685 mg of the title compound is obtained as a pH-dependent 5-(E/Z)-isomer mixture.

1H-NMR (DMSO-d6, stored with K₂CO₃, main isomer): δ=1.18-1.35 (6H); 4.18-4.32 (4H); 4.78 (2H); 7.16 (1H); 7.25-7.41 (2H); 7.45 (1H); 8.20 (1H); 10.60 (1H) ppm.

### EXAMPLE as)

### 4-(3-{[2-((E or Z)-Cyano-ethoxycarbonyl-methylene)-3-ethyl-4-oxo-thiazolidin-5-(E/Z)-ylidenemethyl]-amino}-benzyl)-piperazine-1-carboxylic acid-tert-butyl ester

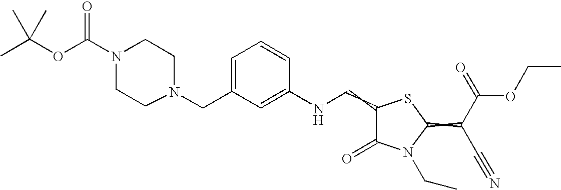

Analogously to Example 225), after purification by chromatography on silica gel, 680 mg of the title compound is obtained as a pH-dependent 5-(E/Z)-isomer mixture from 750 mg of the compound that is described under Example ar), 700 mg of potassium carbonate and 480 mg of 1-tert-butyloxycarbonyl piperazine in 50 ml of DMF.

1H-NMR (DMSO-d6, stored with K₂CO₃, main isomer): δ=1.16-1.32 (6H); 1.40 (9H); 2.21-2.40 (4H); 3.21-3.45 (4H); 3.46 (2H); 4.15-4.33 (4H); 7.04 (1H); 7.16-7.47 (3H); 8.20 (1H); 10.56 (1H) ppm.

### EXAMPLE at)

### (E or Z)-Cyano-{3-ethyl-4-oxo-5-(E/Z)-[(3-piperazin-1-yl-methyl-phenylamino)-methylene]-thiazolidin-2-ylidene}-acetic acid ethyl ester

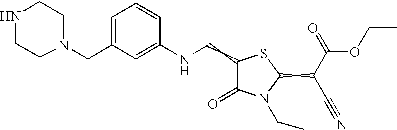

680 mg of the compound, described under Example as), in 20 ml of dichloromethane is mixed with 10 ml of trifluoroacetic acid and stirred for 2 hours at room temperature. The solvent is distilled off in a rotary evaporator, and 850 mg of the title compound is obtained as a pH-dependent 5-(E/Z)-isomer mixture as a crude product.

### EXAMPLE au)

### 2-(4-Amino-phenoxy)-ethanol

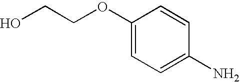

2 g of 2-(4-nitrophenoxy)ethanol is dissolved in 80 ml of THF, mixed with a suspension of 420 mg of palladium on carbon in ethanol and hydrogenated overnight at room temperature under normal pressure. The reaction mixture is filtered on Celite, and after the solvent is distilled off in a rotary evaporator, 1.6 g of the title compound is obtained as a crude product.

¹H-NMR (CDCl₃): δ=3.00-3.70 (3H); 3.85-3.95 (2H); 3.95-4.08 (2H); 6.55-6.70 (2H); 6.70-6.84 (2H) ppm.

### EXAMPLE av)

### (E or Z)-Cyano-(3-ethyl-5-(E/Z)-{[4-(2-iodo-ethoxy)-phenylamino]-methylene}-4-oxo-thiazolidin-2-ylidene)-acetic acid ethyl ester

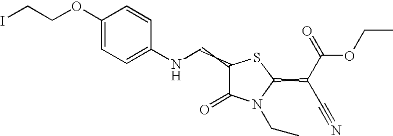

560 mg of the compound that is described under Example 219), 440 mg of triphenylphosphine and 144 mg of imidazole are dissolved in 50 ml of THF, mixed in portions with 426 mg of iodine and stirred overnight at room temperature. The reaction mixture is mixed with water and extracted with ethyl acetate. After purification by chromatography on silica gel, 550 mg of the title compound is obtained as a pH-dependent 5-(E/Z)-isomer mixture.

¹H-NMR (DMSO-d6, stored with K₂CO₃, main isomer): δ=1.18-1.32 (6H); 3.51 (2H); 4.18-4.30 (6H); 6.98 (2H); 7.27 (2H); 8.13 (1H); 10.50 (1H) ppm.

### EXAMPLE aw)

### Cyano-cyclopropylthiocarbamoyl-acetic acid ethyl ester

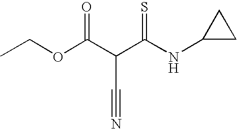

4.85 ml of cyanoacetic acid ethyl ester, 5.24 ml of triethylamine and 5.0 g of cyclopropyl isothiocyanate are stirred overnight at 50° C. The reaction mixture that is obtained is diluted with 10 ml of EtOH and slowly added to 220 ml of 1 M HCl. It is stirred for 2 hours. The precipitate that is produced is suctioned off and washed with water. The solid is dissolved in dichloromethane and washed with saturated aqueous sodium chloride solution. The organic phase is dried (MgSO₄), and the solvent is removed from the product. 6.9 g of the product is obtained.

¹H-NMR (CDCl₃): δ=0.78 (2H), 0.94 (2H), 1.29 (3H), 2.73 (1H), 4.18 (2H), 4.89 (1H), 11.18 (1H) ppm.

### EXAMPLE ax)

### (E or Z)-Cyano-(3-cyclopropyl-4-oxo-thiazolidin-2-ylidene)-acetic acid ethyl ester

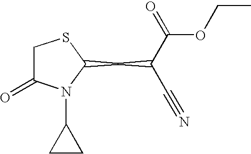

Analogously to Example b), after recrystallization from diethyl ether/hexane, 6.2 g of product is obtained from 6.9 g of the compound that is described under Example ya), and 3.3 ml of bromoacetyl chloride in 210 ml of tetrahydrofuran.

MS (CI/NH₃) m/z=270 (M+H₂O)⁺

### EXAMPLE ay)

### (E or Z)-Cyano-(3-cyclopropyl-5-(E or Z)-ethoxy-methylene-4-oxo-thiazolidin-2-(Z)-ylidene)-acetic acid ethyl ester

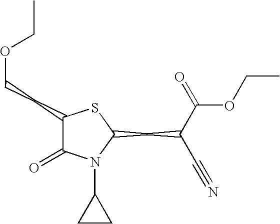

Analogously to Example c), 4.22 g of product is obtained from 6.22 g of the compound that is described under Example yb), 9.61 ml of triethyl orthoformate and 13.46 ml of acetic acid anhydride after stirring with diethyl ether.

¹H-NMR (CDCl₃): δ=1.10 (2H), 1.37 (6H), 1.90 (2H), 3.12 (1H), 4.21 (2H), 4.31 (2H), 7.65 (1H) ppm.

### EXAMPLE az)

### (E or Z)-[Cyano-(3-ethyl-4-oxo-5-(E/Z)-phenylaminomethylene-thiazolidin-2-ylidene)-acetic acid

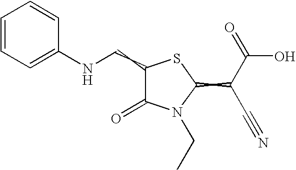

1.50 g of the ester that is produced in Example 1 is dissolved in 19 ml of dioxane, mixed with 7.5 ml of an ethanolic potassium hydroxide solution and then stirred for 18 hours at 25° C. It is diluted with 150 ml of water, acidified with 1N sulfuric acid to pH 2, the solid is suctioned off on one frit and dried in a vacuum at 70° C. The thus obtained product can be used without further purification in the next stage.

¹H-NMR (DMSO-d6, stored with K2CO3, main isomer): δ=1.23 (3H), 4.25 (2H), 7.08 (1H), 7.27-7.42 (4H), 8.14 (1H), 10.42 (1H), 13.05 (1H) ppm.

### EXAMPLE ba)

### (E or Z)-Cyano-(3-ethyl-5-(E/Z)-{[4-(2-iodo-ethyl)-phenylamino]-methylene}-4-oxo-thiazolidin-2-ylidene)-acetic acid ethyl ester

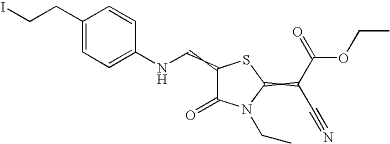

Analogously to Example av), after purification by chromatography on silica gel, 1.06 g of the title compound is obtained as a pH-dependent 5-(E/Z)-isomer mixture from 1.0 g of the compound that is described under Example 459), 817 mg of triphenylphosphine, 267 mg of imidazole and 793 mg of iodine.

1H-NMR (DMSO-d6, stored with K₂CO₃, main isomer): δ=1.19-1.32 (6H); 3.10 (2H); 3.46 (2H); 4.17-4.32 (4H); 7.20-7.31 (4H); 8.20 (1H); 10.51 (1H) ppm.

### EXAMPLE bb)

### (4-Hydroxyphenyl)-carboxylic acid-tert-butyl ester

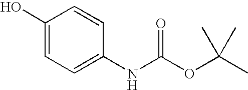

3 g of 4-aminophenol is dissolved in 50 ml of dichloromethane and mixed at 0° C. with 15 ml of diisopropylamine and 6.6 g of di-tert-butyl-dicarbonate and stirred for 18 hours at room temperature. After aqueous working-up and recrystallization from ethyl acetate/hexane, 1.06 g of the title compound is obtained.

### EXAMPLE bc)

### [4-(3-Morpholin-4-yl-propoxy)-phenyl]-carboxylic acid-tert-butylester

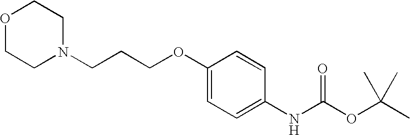

89 mg of the compound that is described under Example bb) is dissolved in 4 ml of butanone and mixed with 130 ml of potassium carbonate, 35 mg of tetrabutylammonium iodide and 100 μl of 4-(3-chloro-propyl)-morpholine and stirred under reflux for 4 hours. After aqueous working-up and purification by chromatography on silica gel, 160 mg of the title compound is obtained.

1H-NMR (DMSO-d6): δ=1.46 (9H); 1.85 (2H); 2.28-2.45 (6H); 3.56 (4H); 3.93 (2H); 6.81 (2H); 7.31 (2H); 9.10 (1H) ppm.

### EXAMPLE bd)

### (3-Amino-phenyl)-carboxylic acid-tert-butyl ester

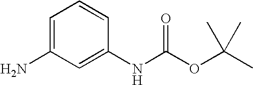

5 g of 1,3-phenylenediamine is dissolved in 50 ml of dichloromethane and mixed at 0° C. with 24 ml of diisopropylamine and 10.8 g of di-tert-butyl-dicarbonate and stirred for 18 hours at room temperature. After aqueous working-up and recrystallization from ethyl acetate/hexane, 4.74 g of the title compound is obtained.

1H-NMR (CDCl₃): δ=1.50 (9H); 3.68 (2H); 6.35 (1H); 6.40 (1H); 6.52 (1H); 6.96 (1H); 7.04 (1H) ppm.

### EXAMPLE be)

### [3-(2-Methoxy-acetylamino)-phenyl]-carboxylic acid-tert-butyl ester

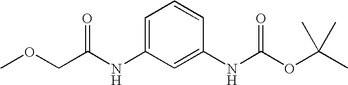

200 mg of the compound that is described under Example bd) is dissolved in 10 ml of tetrahydrofuran and mixed with 400 μl of triethylamine and 136 μl of methoxy-acetyl chloride and stirred for 18 hours at room temperature. After aqueous working-up and purification by chromatography on silica gel, 75 mg of the title compound is obtained.

1H-NMR (DMSO-d6): δ=1.45 (9H); 3.32 (3H); 3.95 (2H); 7.06 (1H); 7.15 (1H); 7.28 (1H); 7.83 (1H); 9.34 (1H); 9.70 (1H) ppm.

### EXAMPLE bf)

### (3-Acryloylamino-phenyl)-carboxylic acid-tert-butyl ester

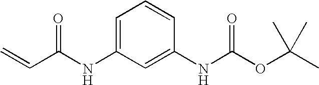

300 mg of the compound that is described under Example bd) is dissolved in 10 ml of tetrahydrofuran and mixed with 400 μl of triethylamine and 156 μl of acrylic acid chloride and stirred for 18 hours at room temperature. After aqueous working-up and purification by chromatography on silica gel, 290 mg of the title compound is obtained.

1H-NMR (DMSO-d6): δ=1.49 (9H); 5.73 (1H); 6.24 (1H); 6.45 (1H); 7.05 (1H); 7.16 (1H); 7.40 (1H); 7.84 (1H); 9.47 (1H); 10.10 (1H) ppm.

### EXAMPLE bg)

### [3-(3-Morpholin-4-yl-propionylamino)-phenyl]-carboxylic acid-tert-butyl ester

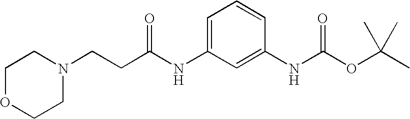

100 mg of the compound that is described under Example bf) is dissolved in 3 ml of tetrahydrofuran and mixed with 158 μl of triethylamine and 50 μl of morpholine and stirred under reflux for 4 hours. After aqueous working-up and purification by chromatography on silica gel, 92 mg of the title compound is obtained.

1H-NMR (DMSO-d6): δ=1.47 (9H); 2.33-2.49 (6H); 2.60 (2H); 3.58 (4H); 7.03 (1H); 7.13 (1H); 7.30 (1H); 7.74 (1H); 9.34 (1H); 10.01 (1H) ppm.

### EXAMPLE bh)

### (3-Ethenesulfonylamino-phenyl)-carboxylic acid-tert-butyl ester

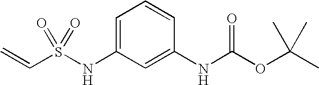

640 mg of the compound that is described under Example bd) is dissolved in 10 ml of tetrahydrofuran and mixed with 1.3 ml of triethylamine and 430 μl of 2-chloroethanesulfonic acid chloride and stirred for 18 hours at room temperature. After aqueous working-up and purification by chromatography on silica gel, 550 mg of the title compound is obtained.

1H-NMR (DMSO-d6): δ=1.46 (9H); 6.04 (1H); 6.11 (1H); 6.65-6.80 (2H); 7.12 (2H); 7.40 (1H); 9.38 (1H); 9.91 (1H) ppm.

### EXAMPLE bi)

### [3-(2-Morpholin-4-yl-ethanesulfonylamino)-phenyl]-carboxylic acid tert-butyl ester

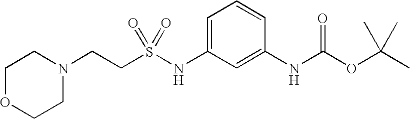

100 mg of the compound that is described under Example bh) is dissolved in 3 ml of tetrahydrofuran and mixed with 13911 of triethylamine and 44 μl of morpholine and stirred under reflux for 12 hours. After aqueous working-up and purification by chromatography on silica gel, 52 mg of the title compound is obtained.

1H-NMR (DMSO-d6): δ=1.46 (9H); 2.30 (4H); 2.55 (2H); 3.21 (2H); 3.48 (4H); 6.78 (1H); 7.04-7.19 (2H); 7.40 (1H); 9.33 (1H); 9.73 (1H) ppm.

The following examples describe the biological action of the compounds according to the invention:

PLK Enzyme Assay

Recombinant human Plk-1 (6×His) was purified from baculovirus-infected insect cells (Hi5).

10 ng of (produced in a recombinant manner and purified) PLK enzyme is incubated for 90 minutes at room temperature with biotinylated casein and 33P-γ-ATP as a substrate in a volume of 15 μl in 384-well Greiner small-volume microtiter plates (final concentrations in the buffer: 660 ng/ml of PLK; 0.7 μmol of casein, 0.5 μmol of ATP incl. 400 nCi/ml of 33P-γ-ATP; 10 mmol of MgCl2, 1 mmol of MnCl2; 0.01% NP40; 1 mmol of DTT, protease inhibitors; 0.1 mmol of Na2VO3 in 50 mmol of HEPES, pH 7.5). To complete the reaction, 5 μl of stop solution (500 μmol of ATP; 500 mmol of EDTA; 1% Triton X100; 100 mg/ml of streptavidin-coated SPA beads in PBS) is added. After the microtiter plate is sealed by film, the beads are sedimented by centrifuging (10 minutes, 1500 rpm). The incorporation of 33P-γ-ATP in casein is intended as a measurement of enzyme activity by β-counting. The extent of the inhibitor activity is referenced against a solvent control (=uninhibited enzyme activity=0% inhibition) and the mean value of several batches that contained 300 mmol of wortmannin (=completely inhibited enzyme activity=100% inhibition).

Test substances are used in various concentrations (0 μmol, as well as in the range of 0.01-30 μmol). The final concentration of the solvent dimethyl sulfoxide is 1.5% in all batches.

Proliferation Assay

Cultivated human MaTu breast tumor cells were flattened out at a density of 5000 cells/measuring point in a 96-well multititer plate in 20011 of the corresponding growth medium. After 24 hours, the cells of one plate (zero-point plate) were colored with crystal violet (see below), while the medium of the other plates was replaced by fresh culture medium (20011), to which the test substances were added in various concentrations (0 μm, as well as in the range of 0.01-30 μm; the final concentration of the solvent dimethyl sulfoxide was 0.5%). The cells were incubated for 4 days in the presence of test substances. The cell proliferation was determined by coloring the cells with crystal violet: the cells were fixed by adding 20 μl/measuring point of an 11% glutaric aldehyde solution for 15 minutes at room temperature. After three washing cycles of the fixed cells with water, the plates were dried at room temperature. The cells were colored by adding 100 μl/measuring point of a 0.1% crystal violet solution (pH was set at 3 by adding acetic acid). After three washing cycles of the colored cells with water, the plates were dried at room temperature. The dye was dissolved by adding 100 μl/measuring point of a 10% acetic acid solution. The extinction was determined by photometry at a wavelength of 595 nm. The change of cell growth, in percent, was calculated by standardization of the measured values to the extinction values of the zero-point plate (=0%) and the extinction of the untreated (0 μm) cells (=100%).

The results of the two assays are presented in the table below:

| | | Inhibition of the Tumor |
|---|---|---|
| | Inhibition of PLK-1 IC50 | Cell Proliferation |
| Compound No. | [nmol] | (MaTu) IC50 [μmol] |
| 10 | 200 | 4 |
| 11 | 400 | 15 |
| 12 | 100 | 2 |
| 21 | 230 | |
| 22 | 510 | |
| 24 | 230 | |
| 26 | 6300 | |
| 28 | 2300 | |
| 30 | 250 | |
| 31 | 1900 | |
| 32 | 1700 | |
| 33 | 420 | |
| 35 | 2800 | |
| 36 | 1800 | |
| 37 | 3500 | |
| 38 | 3000 | |
| 39 | 5900 | |
| 40 | 1700 | |
| 45 | 680 | |
| 47 | 410 | |
| 48 | 270 | |
| 50 | 240 | |
| 52 | 260 | |
| 53 | 460 | |
| 55 | 1500 | |
| 56 | 2200 | |
| 58 | 270 | |
| 59 | 180 | |
| 60 | 290 | |

### DESCRIPTION OF THE FIGURE

FIG. 1 shows the function of Plk-1

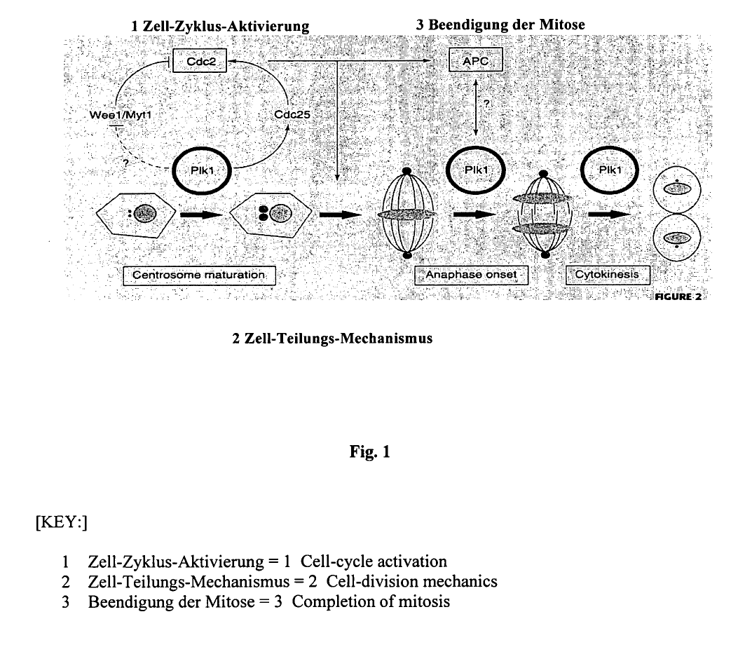

Here:
1. Entry into mitosis: Plk-1-activated CDC25 C. This results in the activation of the CDK/cyclin B complex and converts the cell from G2 to M-status.
2. Triggering of mitosis: Plk 1 plays an important role during the cytokinesis, especially in the formation of the bipolar spindle apparatus and the chromosome separation during the late mitosis phase. Plk-1 is also required during centrosome maturation and binds to so-called ‘kinesin motors.’
3. Completion of mitosis: Plk-1 activates the APC/C complex (anaphase promoting complex/cyclosome; Kotani et al. 1998;). APC/C catalyzes as E3-enzyme the polyubiquitinylation of specific substrates, such as, e.g., cyclin B. Such an ubiquitinylation of proteins results only in their degradation into proteasomes. This in turn leads to a reduction of cell-cycle regulators below a critical value and in the exit from the mitosis phase in the so-called G1-status of the cell (M→G1 transition).

1. Compounds of general formula I 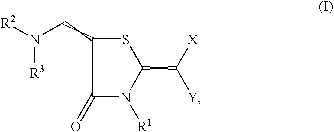
in which
X and Y are the same or different and stand for hydrogen, aryl, cyano, C₃-C₆-cycloalkyl or for the group —COOR⁴, —CONR¹⁵—(CH₂)ₙ—R²⁵, —COOR²⁵, —CONR¹⁵R¹⁶ or —COR¹³,
R¹, R¹¹, R¹²
R¹⁵ R¹⁶
R¹⁹ and R²⁰ are the same or different and stand for hydrogen, C₁-C₁₀-alkyl, C₂-C₁₀ alkenyl, C₂-C₁₀-alkinyl, (COOR¹⁴)—(CH₂)ₙ—, (C₃-C₆-cycloalkyl)-C₁-C₄-alkylene, C₃-C₆-cycloalkyl, phenylsulfonyl, phenyl-C₃-C₆-cycloalkyl, C₁-C₁₀-alkanoyl, C₁-C₆-alkoxy-C₁-C₆-alkylene, C₁-C₄-alkoxycarbonyl-C₁-C₄-alkylene, hydroxy-C₁-C₄-alkylene, —C₁-C₆-alkyl-O—Si(phenyl)₂—C₁-C₆-alkyl, or for the group COOR¹⁴, —COR¹³, —SO₂R¹⁸, —(CH₂)ₙ—NR¹⁵R¹⁶ or —(CH₂)ₙ—C(CH₃)_{q}—(CH₂)ₙNR¹⁵R¹⁶ or —NR¹¹R¹², or 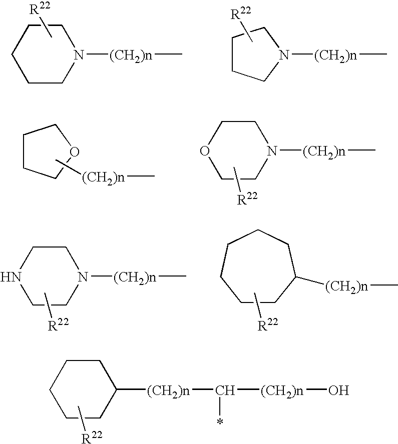
or for aryl, heteroaryl, heterocyclyl, aryl-C₁-C₄-alkylene, heteroaryl-C₁-C₄-alkylene, aryloxy-C₁-C₄-alkylene, heteroaryloxy-C₁-C₄-alkylene or aryl-C₁-C₄-alkylenoxy-C₁-C₄-alkylene that is optionally substituted in one or more places in the same way or differently with C₁-C₆-alkyl, C₂-C₆-alkenyl, C₂-C₆-alkinyl, C₃-C₆-cycloalkyl, C₃-C₆-cycloalkyloxy, phenyl, cyano, halogen, hydroxy, C₁-C₄-alkoxy, phenoxy, benzyloxy, C₁-C₄-alkylsulfanyl, benzylsulfanyl, phenylsulfanyl, dimethylamino, acetylamino, trifluoromethyl, trifluoromethoxy, trifluoromethylsulfanyl, acetyl, —CO—C₁-C₆-alkyl, 1-iminoethyl or nitro, or for C₁-C₁₀-alkyl that is substituted in one or more places with fluorine,
R² and R³ are the same or different and stand for hydrogen, C₁-C₆-alkyl, hydroxy-C₁-C₆-alkylene, C₃-C₆-cyclohexyl or for the group —COOR¹⁴, —CONR¹⁵R¹⁶, —COR¹³, —SO₂R¹⁸, —NR¹¹R¹², —(CH₂)ₙ-A, 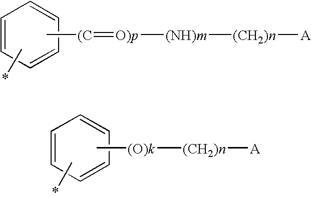
or for aryl, hetaroaryl or heterocyclyl that is optionally substituted in one or more places in the same way or differently with C₁-C₆-alkyl, C₃-C₆-cycloalkyl, halo-C₁-C₆-alkyl, halo-C₁-C₆-alkoxy, halogen, cyano, hydroxy-C₁-C₆-alkylene, hydroxy-C₁-C₆-alkylenoxy, aryl, heteroaryl, heterocyclyl, —C₁-C₆-alkyl-COOR⁸ or with the group —OR¹⁰, —COR¹³, —COOR¹⁴, —NR¹¹R¹², —NR¹¹—CO—NR¹¹R¹²—NR¹¹—CO—R¹³, —NR¹¹—SO₂—R¹³, —(CH₂)ₙ—CO—NR¹⁵R¹⁶, —SR¹⁰ or —SO₂R¹⁸,
R⁴ R⁸, R⁹, R¹⁰,
R¹³, R¹⁴, R¹⁷
and R¹⁸ are the same or different and stand for hydrogen, C₁-C₁₀-alkyl, hydroxy-C₁-C₆-alkylenoxy-C₁-C₆-alkylene, C₁-C₆-alkoxy-CO—C₁-C₆-alkylene, —(CH₂)ₙ-CO—NR¹⁵R¹⁶, C₂-C₁₀-alkenyl, C₂-C₁₀-alkinyl, (C₃-C₆-cycloalkyl)-C₁-C₄-alkylene, halo-C₁-C₆-alkyl, hydroxy-C₁-C₆-alkylene, (COOR¹⁴)—(CH₂)ₙ—, hydroxy-(CH₂)ₙ—O—(CH₂)ₙ, C₃-C₆-cycloalkyl, C₁-C₁₀-alkanoyl, or for the group —NR¹¹R¹², —(CH₂)ₙ—CO—R²⁵, —(CH₂)ₙ—NR¹⁵R¹⁶, COOR¹⁴—(CH₂)ₙ— or —COR¹³, or for aryl, heteroaryl, heterocyclyl, aryl-C₁-C₄-alkylene, heteroaryl-C₁-C₄-alkylene, aryloxy-C₁-C₄-alkylene, heteroaryloxy-C₁-C₄-alkylene or aryl-C₁-C₄-alkylenoxy-C₁-C₄-alkylene that is optionally substituted in one or more places in the same way or differently with C₁-C₆-alkyl, C₂-C₆-alkenyl, C₃-C₆-cycloalkyl, C₃-C₆-cycloalkyloxy, phenyl, cyano, halogen, hydroxy-C₁-C₆-alkyl, C₁-C₄-alkoxy, phenoxy, benzyloxy, C₁-C₄-alkylsulfanyl, benzylsulfanyl, phenylsulfanyl, dimethylamino, acetylamino, trifluoromethyl, trifluoromethoxy, trifluoromethylsulfanyl, acetyl, —CO—C₁-C₆-alkyl, 1-iminoethyl or nitro, or for C₁-C₁₀-alkyl that is substituted in one or more places with fluorine or for the group NR¹¹R¹², —COR¹³, —SO₂R¹⁸, —(CH₂)ₙ—NR¹⁵R¹⁶, —(CH₂)ₙ—C(CH₃)_{q}—(CH₂)ₙNR¹⁵R¹⁶ or 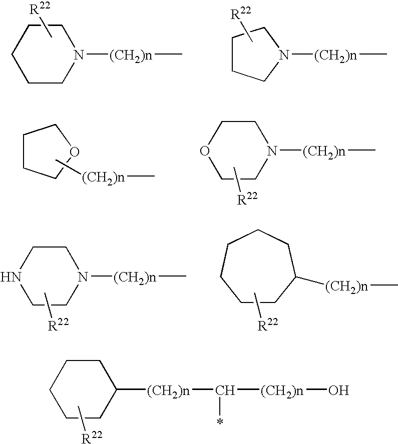
or
R² and R³,
R¹¹ and R¹²,
R¹⁵ and R¹⁶
and
R¹⁹ and R²⁰, in each case independently of one another, together form a 3- to 10-membered ring, which optionally can contain one or more nitrogen, oxygen or sulfur atoms,
or
R³ stands for hydrogen,
and
R² stands for the group -(L-M), in which
L stands for a group —C(O)—, —S(O)₂, —C(O)N(R⁷)—, —S(O)₂N(R⁷)—, —C(S)N(R⁷)—, —C(S)N(R⁷)C(O)O—, —C(O)O— or —C(O)S—, and
M stands for hydrogen, C₁-C₁₀-alkyl, C₂-C₁₀-alkenyl, C₂-C₁₀-alkinyl, (C₃-C₆-cycloalkyl)-C₁-C₄-alkylene, C₃-C₆-cycloalkyl, phenyl-C₃-C₆-cycloalkyl, C₁-C₁₀-alkanoyl, C₁-C₄-alkoxy-C₁-C₄-alkylene, C₁-C₄-alkoxycarbonyl-C₁-C₄-alkylene, hydroxy-C₁-C₁₀-alkylene, or for aryl, heteroaryl, heterocyclyl, aryl-C₁-C₄-alkylene, heteroaryl-C₁-C₄-alkylene, aryloxy-C₁-C₄-alkylene, heteroaryloxy-C₁-C₄-alkylene or aryl-C₁-C₄-alkylenoxy-C₁-C₄-alkylene that is optionally substituted in one or more places in the same way or differently with C₁-C₄-alkyl, C₂-C₆-alkenyl, C₃-C₆-cycloalkyl, C₃-C₆-cycloalkyloxy, phenyl, cyano, halogen, phenoxy, benzyloxy, halo-C₁-C₄-alkoxy, halo-C₁-C₆-alkyl, nitro, —C₁-C₆-alkylCOOR⁸, —C₂-C₆-alkenylCOOR⁸, —C₂-C₆-alkinylCOOR⁸, —C₁-C₆-alkylOR⁹, —C₂-C₆-alkenylOR⁹, —C₁-C₆-alkinylOR⁹ or with the group —OR¹⁰, —NR¹¹R¹², —COR¹³, —COOR¹⁴, —CONR¹⁵R¹⁶, —SR¹⁷, —SO₂R¹⁸, SO₂NR¹⁹R²⁰ or —C(NH)(NH₂), or for C₁-C₁₀-alkyl that is substituted in one or more places with fluorine, and
R⁷ stands for hydrogen, C₁-C₁₀-alkyl, C₂-C₁₀-alkenyl, C₂-C₁₀-alkinyl, C₃-C₆-cycloalkyl, (C₃-C₆-cycloalkyl)-C₁-C₄-alkylene, aryl-C₁-C₄-alkylene,
A stands for optionally substituted aryl, heteroaryl or heterocyclyl,
R²² stands for hydrogen, hydroxy-C₁-C₆-alkyl, or for the group —OR¹⁰, —NR¹¹R¹², —COR¹³, —CONR¹⁵R¹⁶, —SO₂R¹⁸, —NR¹⁵—(C═S)—NR¹⁶—(CH₂)ₙ—R²⁴, —NR¹⁵—(C═O)—NR¹⁶—(CH₂)ₙ—R²⁴,
R²³ stands for hydrogen or C₁-C₆-alkyl,
R²⁴ stands for hydrogen, phenyl, C₁-C₆-alkoxy or for the group —(CH₂)ₙ—COO—C₁-C₆-alkyl,
R²⁵ stands for the group —OR¹⁰ or for C₂-C₆-alkenyl, phenyl, pyridyl, imidazolyl, morpholinyl, piperidinyl, C₃-C₆-cycloalkyl or 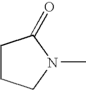
that is optionally substituted in one or more places in the same way or differently with halogen, C₁-C₆-alkyl, hdyroxy-C₁-C₆-alkyl or with the group —OR¹⁰ or —COOR¹⁴,
m, p, k, in each case independently of one another, stand for 0 or 1,
n stands for 0, 1, 2, 3, 4, 5, 6, 7, 8, 9 or 10,
q stands for 1 or 2, as well as their stereoisomers, mixtures of stereosiomers and their salts.

2. Compounds of general formula I, according to claim 1, in which
X and Y are the same or different and stand for hydrogen, phenyl, cyano, C₃-C₆-cycloalkyl or for the group —COOR⁴, —CONR¹⁵—(CH₂)ₙ—R²⁵, —COOR²⁵, —CONR¹⁵R¹⁶ or —COR¹³,
R¹, R¹¹, R¹²
R¹⁵, R¹⁶
R¹⁹ and R²⁰ are the same or different and stand for hydrogen, C₁-C₁₀-alkyl, C₂-C₁₀-alkenyl, C₂-C₁₀-alkinyl, (COOR¹⁴)—(CH₂)ₙ—, (C₃-C₆-cycloalkyl)-C₁-C₄-alkylene, C₃-C₆-cycloalkyl, phenylsulfonyl, phenyl)-C₃-C₆-cycloalkyl, C₁-C₁₀-alkanoyl, C₁-C₆-alkoxy-C₁-C₆-alkylene, C₁-C₄-alkoxycarbonyl-C₁-C₄-alkylene, hydroxy-C₁-C₄-alkylene, —C₁-C₆-alkyl-O—Si(phenyl)₂—C₁-C₆-alkyl, or for the group COOR¹⁴, —COR¹³, —SO₂R¹⁸, —(CH₂)ₙ—NR¹⁵R¹⁶ or —(CH₂)ₙ—C(CH₃)_{q}—(CH₂)ₙNR¹⁵R¹⁶ or —NR¹¹R¹², or 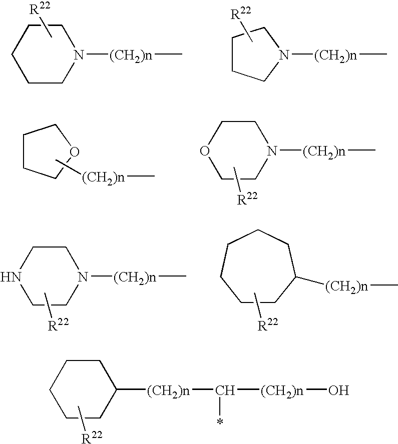
or for aryl, heteroaryl, heterocyclyl, aryl-C₁-C₄-alkylene, heteroaryl-C₁-C₄-alkylene, aryloxy-C₁-C₄-alkylene, heteroaryloxy-C₁-C₄-alkylene or aryl-C₁-C₄-alkylenoxy-C₁-C₄-alkylene that is optionally substituted in one or more places in the same way or differently with C₁-C₆-alkyl, C₂-C₆-alkenyl, C₂-C₆-alkinyl, C₃-C₆-cycloalkyl, C₃-C₆-cycloalkyloxy, phenyl, cyano, halogen, hydroxy, C₁-C₄-alkoxy, phenoxy, benzyloxy, C₁-C₄-alkylsulfanyl, benzylsulfanyl, phenylsulfanyl, dimethylamino, acetylamino, trifluoromethyl, trifluoromethoxy, trifluoromethylsulfanyl, acetyl, —CO—C₁-C₆-alkyl, 1-iminoethyl or nitro, or for C₁-C₁₀-alkyl that is substituted in one or more places with fluorine,
R² and R³ are the same or different and stand for hydrogen, C₁-C₆-alkyl, hydroxy-C₁-C₆-alkylene, C₃-C₆-cyclohexyl or for the group-COOR¹⁴, —CONR¹⁵R¹⁶, —COR¹³, —SO₂R¹⁸, —NR¹¹R¹², —(CH₂)ₙ-A, 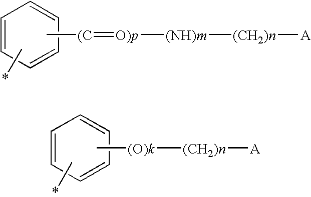
or for aryl, heteroaryl or heterocyclyl that is optionally substituted in one or more places in the same way or differently with C₁-C₆-alkyl, C₃-C₆-cycloalkyl, halo-C₁-C₆-alkyl, halo-C₁-C₆-alkoxy, halogen, cyano, hydroxy-C₁-C₆-alkylene, hydroxy-C₁-C₆-alkylenoxy, aryl, heteroaryl, heterocyclyl, —C₁-C₆-alkyl-COOR⁸ or with the group —OR¹⁰, —COR¹³, —COOR¹⁴, —NR¹¹R¹², —NR¹¹—CO—NR¹¹R¹², —NR¹¹—CO—R¹³, —NR¹¹—SO₂—R¹³, —(CH₂)ₙ—CO—NR¹⁵R¹⁶—SR¹⁰ or —SO₂R¹⁸,
R⁴, R⁸, R⁹,
R¹⁰, R¹³,
R¹⁴, R¹⁷
and R¹⁸ are the same or different and stand for hydrogen, C₁-C₁₀-alkyl, hydroxy-C₁-C₆-alkylenoxy-C₁-C₆-alkylene, C₁-C₆-alkoxy-CO—C₁-C₆-alkylene, —(CH₂)ₙ—CO—NR¹⁵R¹⁶, C₂-C₁₀-alkenyl, C₂-C₁₀-alkinyl, (C₃-C₆-cycloalkyl>C₁-C₄-alkylene, halo-C₁-C₆-alkyl, hydroxy-C₁-C₆-alkylene, (COOR¹⁴)—(CH₂)ₙ—, hydroxy-(CH₂)ₙ—O—(CH₂)ₙ, C₃-C₆-cycloalkyl, C₁-C₁₀-alkanoyl, or for the group —NR¹¹R¹², —(CH₂)ₙ—CO—R²⁵, —(CH₂)ₙ—NR¹⁵R¹⁶, COOR¹⁴—(CH₂)ₙ— or —COR¹³, or for aryl, heteroaryl, heterocyclyl, aryl-(C₁-C₄-alkylene, heteroaryl-C₁-C₄-alkylene, aryloxy-C₁-C₄-alkylene, heteroaryloxy-C₁-C₄-alkylene or aryl-C₁-C₄-alkylenoxy-C₁-C₄-alkylene that is optionally substituted in one or more places in the same way or differently with C₁-C₆-alkyl, C₂-C₆-alkenyl, C₃-C₆-cycloalkyl, C₃-C₆-cycloalkyloxy, phenyl, cyano, halogen, hydroxy-C₁-C₆-alkyl, C₁-C₄-alkoxy, phenoxy, benzyloxy, C₁-C₄-alkylsulfanyl, benzylsulfanyl, phenylsulfanyl, dimethylamino, acetylamino, trifluoromethyl, trifluoromethoxy, trifluoromethylsulfanyl, acetyl, —CO—C₁-C₆-alkyl, 1-iminoethyl or nitro, or for C₁-C₁₀-alkyl that is substituted in one or more places with fluorine or for the group —NR¹¹R¹², —COR¹³, —SO₂R¹⁸, —(CH₂)ₙ—NR¹⁵R¹⁶, —(CH₂)ₙ—C(CH₃)_{q}—(CH₂)ₙNR¹⁵R¹⁶ or 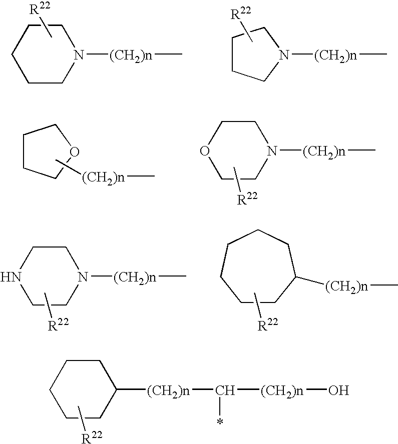
or
R² and R³,
R¹¹ and R¹²,
R¹⁵ and R¹⁶
and
R¹⁹ and R²⁰, in each case independently of one another, together form a 3- to 10-membered ring, which optionally can contain one or more nitrogen, oxygen or sulfur atoms,
A stands for optionally substituted aryl, heteroaryl or heterocyclyl,
R²² stands for hydrogen, hydroxy-C₁-C₆-alkyl, or for the group —OR¹⁰, —NR¹¹R¹², —COR¹³, —CONR¹⁵R¹⁶, —SO₂R¹⁸, —NR¹⁵—(C═S)—NR¹⁶—(CH₂)ₙ—R²⁴, —NR¹⁵—(C═O)—NR¹⁶—(CH₂)ₙ—R²⁴,
R²³ stands for hydrogen or C₁-C₆-alkyl,
R²⁴ stands for hydrogen, phenyl, C₁-C₆-alkoxy or for the group —(CH₂)ₙ—COO—C₁-C₆-alkyl,
R²⁵ stands for the group —OR¹⁰ or for C₂-C₆-alkenyl, phenyl, pyridyl, imidazolyl, morpholinyl, piperidinyl, C₃-C₆-cycloalkyl or 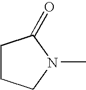
that is optionally substituted in one or more places in the same way or differently with halogen, C₁-C₆-alkyl, hydroxy-C₁-C₆-alkyl or with the group —OR¹⁰ or —COOR¹⁴,
m, p, k, in each case independently of one another, stand for 0 or 1,
n stands for 0, 1, 2, 3, 4, 5, 6, 7, 8, 9 or 10,
q stands for 1 or 2, as well as their stereoisomers, mixtures of the stereoisomers and their salts.

3. Compounds of general formula 1, according to claim 1, in which
X and Y are the same or different and stand for hydrogen, phenyl, cyano, C₃-C₆-cycloalkyl or for the group —COOR⁴, —CONR¹⁵—(CH₂)ₙ—R²⁵, —COOR²⁵, —CONR¹⁵R¹⁶ or —COR¹³,
R¹ stands for hydrogen, phenyl, C₁-C₆-alkyl, C₃-C₆-cycloalkyl, hydroxy-C₁-C₄-alkylene, C₁-C₆-alkoxy-C₁-C₆-alkylene or for the group —C₁-C₆-alkyl-O—Si(phenyl)₂—C₁-C₆-alkyl,
R² and R³ are the same or different and stand for hydrogen, C₁-C₆-alkyl, hydroxy-C₁-C₄-alkylene, cyclohexyl or for the group-COOR¹⁴, —CONR¹⁵R¹⁶, —COR¹³, —SO₂R¹⁸, —NR¹¹R¹², —(CH₂)ₙ-A 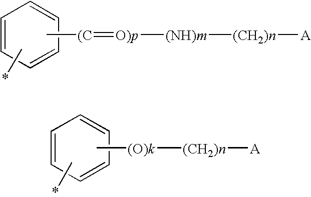
or for phenyl, pyridyl, naphthyl, biphenyl, imidazolyl, indazolyl, isothiazolyl, triazolyl, benztriazolyl, quinolinyl, isoquinolinyl, thiazolyl, pyrazolyl, anthrazenyl, pyrazolidinyl, oxazolyl, phthalazinyl, carbazolyl, benzimidazolyl, benzthiazolyl, isoxazolyl, indanyl, indolyl, pyrimidinyl, thiadiazolyl or 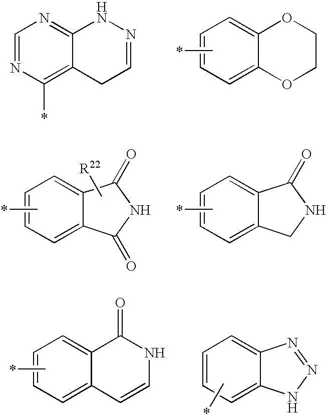
that is optionally substituted in one or more places in the same way or differently with C₁-C₆-alkyl, C₃-C₆-cycloalkyl, halo-C₁-C₆-alkyl, halo-C₁-C₆-alkoxy, halogen, cyano, triazolyl, tetrazolyl, hydroxy-C₁-C₆-alkylene, hydroxy-C₁-C₆-alkylenoxy, morpholino, —C₁-C₆-alkyl-COOR⁸ or with the group —OR¹⁰, —COR¹³, —COOR¹⁴, —NR¹¹R¹², —NR¹¹—CO—NR¹¹R¹², —NR¹¹—CO—R¹³, —NR¹¹—SO₂—R¹³, —(CH₂)ₙ—CO—NR¹⁵R¹⁶, —SR¹⁰ or —SO₂R¹⁸
or
R² and R³ together form a piperidino or morpholino ring,
A stands for the group 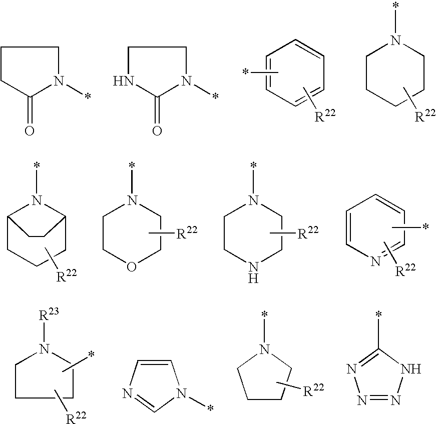
R⁴ stands for hydrogen, C₁-C₆-alkyl, halo-C₁-C₆-alkyl, hydroxy-C₁-C₆-alkyl, hydroxy-(CH₂)ₙ—O—(CH₂)ₙ—, or for the group —(CH₂)ₙ—CO—R²⁵, —(CH₂)ₙ—NR¹⁵R¹⁶, or for phenyl or benzyl that is optionally substituted with hydroxy-C₁-C₆-alkyl,
R⁸, R¹¹,
R¹², R¹⁴, R¹⁵
and R¹⁶ are the same or different and stand for hydrogen, C₁-C₁₀-alkyl, hydroxy-C₁-C₆-alkylene, (COOR¹⁴)—(CH₂)ₙ— or for phenyl, pyridyl, or pyrimidinyl that is optionally substituted with halogen or with the group —CO—C₁-C₆-alkyl, or for the group —COR¹³, —SO₂R¹⁸, —(CH₂)ₙ—NR¹⁵R¹⁶—(CH₂)ₙ—C(CH₃)_{q}—(CH₂)ₙNR¹⁵R¹⁶ or 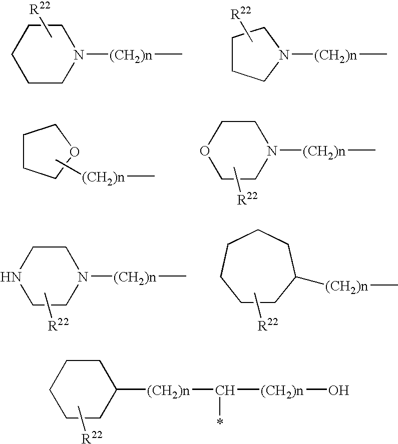
R¹⁰ stands for hydrogen, C₁-C₁₀-alkyl, hydroxy-C₁-C₆-alkylene, hydroxy-C₁-C₆-alkylenoxy-C₁-C₆-alkylene, C₁-C₆-alkoxy-CO—C₁-C₆-alkylene, —(CH₂)ₙ—CO—NR¹⁵R¹⁶ or for phenyl that is optionally substituted with halogen or with the group —CO—C₁-C₆-alkyl, or for the group —COR¹³, —SO₂R¹⁸, COOR¹⁴—(CH₂)ₙ—,
R¹³ stands for hydrogen, C₁-C₁₀-alkyl, C₁-C₁₀-alkenyl, C₁-C₁₀-alkinyl, C₁-C₆-alkyloxy-C₁-C₆-alkenyl, C₁-C₆-alkyloxy-C₁-C₆-alkenyloxy-C₁-C₆-alkenyl, phenyl or for the group 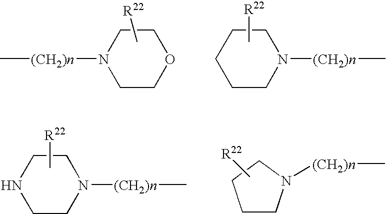
R¹⁸ stands for C₁-C₁₀-alkyl, hydroxy, hydroxy-C₁-C₆-alkyl or for the group —NR¹¹R¹² 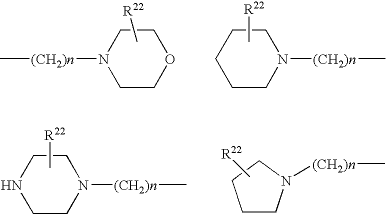
or for phenyl that is optionally substituted in one or more places in the same way or differently with C₁-C₆-alkyl,
R²² stands for hydrogen, hydroxy-C₁-C₆-alkyl, or for the group —OR¹⁰, —NR¹¹R¹², —COR¹³, —CONR¹⁵R¹⁶, —SO₂R¹⁸, —NR¹⁵—(C═S)—NR¹⁶—(CH₂)ₙ—R²⁴, —NR¹⁵—(C═O)—NR¹⁶—(CH₂)ₙ—R²⁴,
R²³ stands for hydrogen or C₁-C₆-alkyl,
R²⁴ stands for hydrogen, phenyl, C₁-C₆-alkoxy or for the group —(CH₂)ₙ—COO—C₁-C₆-alkyl,
R²⁵ stands for the group —OR¹⁰ or for C₂-C₆-alkenyl, phenyl, pyridyl, imidazolyl, morpholinyl, piperidinyl, C₃-C₆-cycloalkyl or 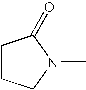
that is optionally substituted in one or more places in the same way or differently with halogen, C₁-C₆-alkyl, hydroxy-C₁-C₆-alkyl or with the group —OR¹⁰ or —COOR¹⁴,
m, p, k, in each case independently of one another, stand for 0 or 1,
n stands for 0, 1, 2, 3, 4, 5, 6, 7, 8, 9 or 10,
q stands for 1 or 2, as well as their stereoisomers, mixtures of the stereoisomers and their salts.

4. Compounds of general formulas II and III, 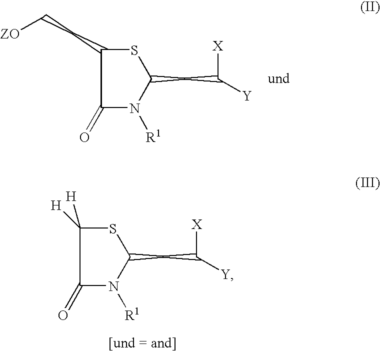
in which X, Y and R¹ have the meanings that are indicated in general formula I, and Z stands for C₁-C₁₀-alkyl, as intermediate products for the production of the compounds of general formula I according to the invention.

5. Intermediate compounds of general formula II, according to claim 4, in which Z stands for C₁-C₄ alkyl.

6. Use of the compounds of general formula I, according to claim 1, for the production of a pharmaceutical agent for treating cancer, auto-immune diseases, chemotherapy agent-induced alopecia and mucositis, cardiovascular diseases, infectious diseases, nephrological diseases, chronic and acute neurodegenerative diseases and viral infections.

7. Use according to claim 6, characterized in that cancer is defined as solid tumors and leukemia; auto-immune diseases are defined as psoriasis, alopecia and multiple sclerosis; cardiovascular diseases are defined as stenoses, arterioscleroses and restenoses; infectious diseases are defined as diseases that are caused by unicellular parasites; nephrological diseases are defined as glomerulonephritis; chronic neurodegenerative diseases are defined as Huntington's disease, amyotrophic lateral sclerosis, Parkinson's disease, AIDS dementia and Alzheimer's disease; acute neurodegenerative diseases are defined as ischemias of the brain and neurotraumas; and viral infections are defined as cytomegalic infections, herpes, hepatitis B and C, and HIV diseases.

8. Pharmaceutical agents that contain at least one compound according to claim 1.

9. Pharmaceutical agents according to claim 8 for treating cancer, autoimmune diseases, cardiovascular diseases, infectious diseases, nephrological diseases, neurodegenerative diseases and viral infections.

10. A composition comprising a compound according to claim 1 with one or more suitable formulation substances and/or vehicles.

11. Use of the compounds of general formula I and the pharmaceutical agents, according to claim 1, as inhibitors of the polo-like kinases.

12. Use according to claim 11, wherein the kinase is Plk1, Plk2, Plk3 or Plk4.

13. Use of the compounds of general formula I, according to claim 1, in the form of a pharmaceutical preparation for enteral, parenteral and oral administration.
